SEQUENCE LISTING (417 sequences)

SEQ ID NO: 1 - protein, 832 aa, Homo sapiens
MALPVTALLLPLALLLHAARPDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSPRASTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 2 - protein, 1234 aa, Homo sapiens
MALPVTALLLPLALLLHAARPDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSPRASTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGEGGGGGDLGGVKLPHLFGKRLVEARMASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTLLLDRHPIAVMLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 3 - protein, 831 aa, Homo sapiens
MALPVTALLLPLALLLHAARPDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSPRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 4 - protein, 1233 aa, Homo sapiens
MALPVTALLLPLALLLHAARPDIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRGGGGSGGGGSGGGGSGGGGSEVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSPRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGEGGGGGDLGGVKLPHLFGKRLVEARMASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTLLLDRHPIAVMLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 5 - protein, 1248 aa, Homo sapiens
MALPVTALLLPLALLLHAARPGSDIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRGGGGSGGGGSGGGGSGGGGSGGGGSEVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSPRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGEGGGGGFKRDLGGVKLPHLFGKRLVEARMASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTLLLDRHPIAVMLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 6 - protein, 841 aa, Homo sapiens
MALPVTALLLPLALLLHAARPDIVMTQAAPSVPVTPGESVSISCRSSKTLLHSNGNTYLYWFLQRPGQSPQVLIYRMSNLASGVPNRFSGSGSETTFTLRISRVEAEDVGIYYCMQHLEYPYTFGGGTKLEIERGGGGSGGGGSGGGGSGGGGSEVQLEESGAELVRPGTSVKLSCKASGYTFTSYWMHWIKQRPEQGLEWIGRIDPYDSETHYNEKFKDKAILSVDKSSSTAYIQLSSLTSDDSAVYYCSRRDAKYDGYALDYWGQGTSVTVSSPRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 7 - protein, 842 aa, Homo sapiens
MALPVTALLLPLALLLHAARPDIVMTQAAPSVPVTPGESVSISCRSSKTLLHSNGNTYLYWFLQRPGQSPQVLIYRMSNLASGVPNRFSGSGSETTFTLRISRVEAEDVGIYYCMQHLEYPYTFGGGTKLEIERGGGGSGGGGSGGGGSGGGGSEVQLEESGAELVRPGTSVKLSCKASGYTFTSYWMHWIKQRPEQGLEWIGRIDPYDSETHYNEKFKDKAILSVDKSSSTAYIQLSSLTSDDSAVYYCSRRDAKYDGYALDYWGQGTSVTVSSPRASTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 8 - protein, 835 aa, Homo sapiens
MALPVTALLLPLALLLHAARPGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRPRASTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRTDGSGATNFSLLKQAGDVEENPGPVSEAMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPI

SEQ ID NO: 9 - RNA, 100 nt, Homo sapiens
gaccaaucugacaugcugcaguuuuagagcuagaaauagcaaguuaaaauaaggcuaguccguuaucaacuugaaaaaguggcaccgagucggugcuuuu

SEQ ID NO: 10 - RNA, 100 nt, Homo sapiens
aauucauccugagaugagguguuuuagagcuagaaauagcaaguuaaaauaaggcuaguccguuaucaacuugaaaaaguggcaccgagucggugcuuuu

SEQ ID NO: 11 - RNA, 100 nt, Homo sapiens
cauccugagaugagguggguguuuuagagcuagaaauagcaaguuaaaauaaggcuaguccguuaucaacuugaaaaaguggcaccgagucggugcuuuu

SEQ ID NO: 12 - RNA, 100 nt, Homo sapiens
guagacauugaccuccgugaguuuuagagcuagaaauagcaaguuaaaauaaggcuaguccguuaucaacuugaaaaaguggcaccgagucggugcuuuu

SEQ ID NO: 13 - RNA, 100 nt, Homo sapiens
aucacggaggucaaugucuaguuuuagagcuagaaauagcaaguuaaaauaaggcuaguccguuaucaacuugaaaaaguggcaccgagucggugcuuuu

SEQ ID NO: 14 - RNA, 100 nt, Homo sapiens
gagaaucaaaaucggugaauguuuuagagcuagaaauagcaaguuaaaauaaggcuaguccguuaucaacuugaaaaaguggcaccgagucggugcuuuu

SEQ ID NO: 15 - RNA, 100 nt, Homo sapiens
acagcugacaggcucgacacguuuuagagcuagaaauagcaaguuaaaauaaggcuaguccguuaucaacuugaaaaaguggcaccgagucggugcuuuu

SEQ ID NO: 16 - RNA, 100 nt, Homo sapiens
agggcaugucaauauuacugguuuuagagcuagaaauagcaaguuaaaauaaggcuaguccguuaucaacuugaaaaaguggcaccgagucggugcuuuu

SEQ ID NO: 17 - RNA, 100 nt, Homo sapiens
cguuccaacucgaagugccaguuuuagagcuagaaauagcaaguuaaaauaaggcuaguccguuaucaacuugaaaaaguggcaccgagucggugcuuuu

SEQ ID NO: 18 - protein, 19 aa, Homo sapiens
MALPVTALLLPLALLLHAA

SEQ ID NO: 19 - protein, 11 aa, Homo sapiens
MALPVTALLLP

SEQ ID NO: 20 - protein, 12 aa, Homo sapiens
PVTALLLPLALL

SEQ ID NO: 21 - protein, 14 aa, Homo sapiens
LLLPLALLLHAARP

SEQ ID NO: 22 - DNA, 23 bp, Homo sapiens
  misc_feature (21)..(21): n is a, c, g, or t
ttgacatgccctcagtatccngg

SEQ ID NO: 23 - DNA, 23 bp, Homo sapiens
  misc_feature (21)..(21): n is a, c, g, or t
ctggattacctcttgccctcngg

SEQ ID NO: 24 - DNA, 23 bp, Homo sapiens
  misc_feature (21)..(21): n is a, c, g, or t
gagatggagactttatatgcngg

SEQ ID NO: 25 - DNA, 23 bp, Homo sapiens
  misc_feature (21)..(21): n is a, c, g, or t
agatggagactttatatgctngg

SEQ ID NO: 26 - DNA, 23 bp, Homo sapiens
  misc_feature (21)..(21): n is a, c, g, or t
agggcatgtcaatattactgngg

SEQ ID NO: 27 - DNA, 23 bp, Homo sapiens
  misc_feature (21)..(21): n is a, c, g, or t
gatggagactttatatgctgngg

SEQ ID NO: 28 - DNA, 23 bp, Homo sapiens
  misc_feature (21)..(21): n is a, c, g, or t
tattatgtctgctaccccagngg

SEQ ID NO: 29 - DNA, 23 bp, Homo sapiens
  misc_feature (21)..(21): n is a, c, g, or t
tgccatagtatttcagatccngg

SEQ ID NO: 30 - DNA, 23 bp, Homo sapiens
  misc_feature (21)..(21): n is a, c, g, or t
agataaaagttcgcatcttcngg

SEQ ID NO: 31 - DNA, 23 bp, Homo sapiens
  misc_feature (21)..(21): n is a, c, g, or t
ctgaaaattccttcagtgacngg

SEQ ID NO: 32 - DNA, 23 bp, Homo sapiens
  misc_feature (21)..(21): n is a, c, g, or t
ctgagggcaagaggtaatccngg

SEQ ID NO: 33 - DNA, 23 bp, Homo sapiens
  misc_feature (21)..(21): n is a, c, g, or t
tttcagatccaggatactgangg

SEQ ID NO: 34 - DNA, 23 bp, Homo sapiens
  misc_feature (21)..(21): n is a, c, g, or t
tatctctacctgagggcaagngg

SEQ ID NO: 35 - DNA, 23 bp, Homo sapiens
  misc_feature (21)..(21): n is a, c, g, or t
tgaggatcacctgtcactgangg

SEQ ID NO: 36 - DNA, 23 bp, Homo sapiens
  misc_feature (21)..(21): n is a, c, g, or t
aatcatctgctacggacaacngg

SEQ ID NO: 37 - DNA, 23 bp, Homo sapiens
  misc_feature (21)..(21): n is a, c, g, or t
gcagacttttgacgcttgacngg

SEQ ID NO: 38 - DNA, 23 bp, Homo sapiens
  misc_feature (21)..(21): n is a, c, g, or t
ccgttccaactcgaagtgccngg

SEQ ID NO: 39 - DNA, 23 bp, Homo sapiens
  misc_feature (21)..(21): n is a, c, g, or t
cgttccaactcgaagtgccangg

SEQ ID NO: 40 - DNA, 23 bp, Homo sapiens
  misc_feature (21)..(21): n is a, c, g, or t
ctggcacttcgagttggaacngg

SEQ ID NO: 41 - DNA, 23 bp, Homo sapiens
  misc_feature (21)..(21): n is a, c, g, or t
gtctgccagcggctgaactgngg

SEQ ID NO: 42 - DNA, 23 bp, Homo sapiens
  misc_feature (21)..(21): n is a, c, g, or t
atcatctgctacggacaactngg

SEQ ID NO: 43 - DNA, 23 bp, Homo sapiens
  misc_feature (21)..(21): n is a, c, g, or t
agacttttgacgcttgactgngg

SEQ ID NO: 44 - DNA, 23 bp, Homo sapiens
  misc_feature (21)..(21): n is a, c, g, or t
cagacttttgacgcttgactngg

SEQ ID NO: 45 - DNA, 23 bp, Homo sapiens
  misc_feature (21)..(21): n is a, c, g, or t
cctggcacttcgagttggaangg

SEQ ID NO: 46 - DNA, 23 bp, Homo sapiens
  misc_feature (21)..(21): n is a, c, g, or t
gcaccccacagttcagccgcngg

SEQ ID NO: 47 - DNA, 23 bp, Homo sapiens
  misc_feature (21)..(21): n is a, c, g, or t
ccttgaggtagacctccagcngg

SEQ ID NO: 48 - DNA, 23 bp, Homo sapiens
  misc_feature (21)..(21): n is a, c, g, or t
aggtctacctcaaggacggangg

SEQ ID NO: 49 - DNA, 23 bp, Homo sapiens
  misc_feature (21)..(21): n is a, c, g, or t
tggaacgggtgagccttgccngg

SEQ ID NO: 50 - DNA, 23 bp, Homo sapiens
  misc_feature (21)..(21): n is a, c, g, or t
tgtggggtgcccttaagcctngg

SEQ ID NO: 51 - DNA, 23 bp, Homo sapiens
  misc_feature (21)..(21): n is a, c, g, or t
aagcgtcaaaagtctgccagngg

SEQ ID NO: 52 - DNA, 23 bp, Homo sapiens
  misc_feature (21)..(21): n is a, c, g, or t
tagcagatgattgagctctgngg

SEQ ID NO: 53 - DNA, 23 bp, Homo sapiens
  misc_feature (21)..(21): n is a, c, g, or t
gattgagctctgaggtgtgtngg

SEQ ID NO: 54 - DNA, 23 bp, Homo sapiens
  misc_feature (21)..(21): n is a, c, g, or t
ggggccggagctccaagcagngg

SEQ ID NO: 55 - DNA, 23 bp, Homo sapiens
  misc_feature (21)..(21): n is a, c, g, or t
ggtgtgtaggtgacaaggaangg

SEQ ID NO: 56 - DNA, 23 bp, Homo sapiens
  misc_feature (21)..(21): n is a, c, g, or t
ccggagctccaagcagtgggngg

SEQ ID NO: 57 - DNA, 23 bp, Homo sapiens
  misc_feature (21)..(21): n is a, c, g, or t
ggtagacctccagctggcccngg

SEQ ID NO: 58 - DNA, 23 bp, Homo sapiens
  misc_feature (21)..(21): n is a, c, g, or t
ctcgaagtgccagggccagcngg

SEQ ID NO: 59 - DNA, 23 bp, Homo sapiens
  misc_feature (21)..(21): n is a, c, g, or t
ctggccctggcacttcgagtngg

SEQ ID NO: 60 - DNA, 23 bp, Homo sapiens
  misc_feature (21)..(21): n is a, c, g, or t
tctgccagcggctgaactgtngg

SEQ ID NO: 61 - DNA, 23 bp, Homo sapiens
  misc_feature (21)..(21): n is a, c, g, or t
ccatgtgccatccgtccttgngg

SEQ ID NO: 62 - DNA, 23 bp, Homo sapiens
  misc_feature (21)..(21): n is a, c, g, or t
ccagctggaggtctacctcangg

SEQ ID NO: 63 - DNA, 23 bp, Homo sapiens
  misc_feature (21)..(21): n is a, c, g, or t
tctgaggtgtgtaggtgacangg

SEQ ID NO: 64 - DNA, 23 bp, Homo sapiens
  misc_feature (21)..(21): n is a, c, g, or t
aggaaggggccaaggcttaangg

SEQ ID NO: 65 - DNA, 23 bp, Homo sapiens
  misc_feature (21)..(21): n is a, c, g, or t
cagagctcaatcatctgctangg

SEQ ID NO: 66 - DNA, 23 bp, Homo sapiens
  misc_feature (21)..(21): n is a, c, g, or t
gggccggagctccaagcagtngg

SEQ ID NO: 67 - DNA, 23 bp, Homo sapiens
  misc_feature (21)..(21): n is a, c, g, or t
cctcccactgcttggagctcngg

SEQ ID NO: 68 - DNA, 23 bp, Homo sapiens
  misc_feature (21)..(21): n is a, c, g, or t
tggagctccggccccagctcngg

SEQ ID NO: 69 - DNA, 23 bp, Homo sapiens
  misc_feature (21)..(21): n is a, c, g, or t
gtgtgtaggtgacaaggaagngg

SEQ ID NO: 70 - DNA, 23 bp, Homo sapiens
  misc_feature (21)..(21): n is a, c, g, or t
atggtttgcagccagagctgngg

SEQ ID NO: 71 - DNA, 23 bp, Homo sapiens
  misc_feature (21)..(21): n is a, c, g, or t
ctggaggtctacctcaaggangg

SEQ ID NO: 72 - DNA, 23 bp, Homo sapiens
  misc_feature (21)..(21): n is a, c, g, or t
ctgccagcggctgaactgtgngg

SEQ ID NO: 73 - DNA, 23 bp, Homo sapiens
  misc_feature (21)..(21): n is a, c, g, or t
aatgacatgtgtcactctctngg

SEQ ID NO: 74 - DNA, 23 bp, Homo sapiens
  misc_feature (21)..(21): n is a, c, g, or t
acatggtttgcagccagagcngg

SEQ ID NO: 75 - DNA, 23 bp, Homo sapiens
  misc_feature (21)..(21): n is a, c, g, or t
catggtttgcagccagagctngg

SEQ ID NO: 76 - DNA, 23 bp, Homo sapiens
  misc_feature (21)..(21): n is a, c, g, or t
gacacatgtcatttctgctgngg

SEQ ID NO: 77 - DNA, 23 bp, Homo sapiens
  misc_feature (21)..(21): n is a, c, g, or t
actggggtcctcccactgctngg

SEQ ID NO: 78 - DNA, 23 bp, Homo sapiens
  misc_feature (21)..(21): n is a, c, g, or t
cctcaaggacggatggcacangg

SEQ ID NO: 79 - DNA, 23 bp, Homo sapiens
  misc_feature (21)..(21): n is a, c, g, or t
aggtgtgtaggtgacaaggangg

SEQ ID NO: 80 - DNA, 23 bp, Homo sapiens
  misc_feature (21)..(21): n is a, c, g, or t
aaggaaggggccaaggcttangg

SEQ ID NO: 81 - DNA, 23 bp, Homo sapiens
  misc_feature (21)..(21): n is a, c, g, or t
gaagtgccagggccagctggngg

SEQ ID NO: 82 - DNA, 23 bp, Homo sapiens
  misc_feature (21)..(21): n is a, c, g, or t
tttgcagccagagctggggcngg

SEQ ID NO: 83 - DNA, 23 bp, Homo sapiens
  misc_feature (21)..(21): n is a, c, g, or t
aaatgacatgtgtcactctcngg

SEQ ID NO: 84 - DNA, 23 bp, Homo sapiens
  misc_feature (21)..(21): n is a, c, g, or t
aggtgacaaggaaggggccangg

SEQ ID NO: 85 - DNA, 23 bp, Homo sapiens
  misc_feature (21)..(21): n is a, c, g, or t
atttctgctgtggctgcagtngg

SEQ ID NO: 86 - DNA, 23 bp, Homo sapiens
  misc_feature (21)..(21): n is a, c, g, or t
gctgtggctgcagttggagangg

SEQ ID NO: 87 - DNA, 23 bp, Homo sapiens
  misc_feature (21)..(21): n is a, c, g, or t
ggcgggggccttgtcgttggngg

SEQ ID NO: 88 - DNA, 23 bp, Homo sapiens
  misc_feature (21)..(21): n is a, c, g, or t
ctctggagttgtggtgggcgngg

SEQ ID NO: 89 - DNA, 23 bp, Homo sapiens
  misc_feature (21)..(21): n is a, c, g, or t
tctggagttgtggtgggcggngg

SEQ ID NO: 90 - DNA, 23 bp, Homo sapiens
  misc_feature (21)..(21): n is a, c, g, or t
cgttggaggtgttgtcttctngg

SEQ ID NO: 91 - DNA, 23 bp, Homo sapiens
  misc_feature (21)..(21): n is a, c, g, or t
agacaacacctccaacgacangg

SEQ ID NO: 92 - DNA, 23 bp, Homo sapiens
  misc_feature (21)..(21): n is a, c, g, or t
gtgggcgggggccttgtcgtngg

SEQ ID NO: 93 - DNA, 23 bp, Homo sapiens
  misc_feature (21)..(21): n is a, c, g, or t
tcgttggaggtgttgtcttcngg

SEQ ID NO: 94 - DNA, 23 bp, Homo sapiens
  misc_feature (21)..(21): n is a, c, g, or t
accacaactccagagcccacngg

SEQ ID NO: 95 - DNA, 23 bp, Homo sapiens
  misc_feature (21)..(21): n is a, c, g, or t
gctctggagttgtggtgggcngg

SEQ ID NO: 96 - DNA, 23 bp, Homo sapiens
  misc_feature (21)..(21): n is a, c, g, or t
gtgggctctggagttgtggtngg

SEQ ID NO: 97 - DNA, 23 bp, Homo sapiens
  misc_feature (21)..(21): n is a, c, g, or t
tgtgggctctggagttgtggngg

SEQ ID NO: 98 - DNA, 23 bp, Homo sapiens
  misc_feature (21)..(21): n is a, c, g, or t
gttggaggtgttgtcttctgngg

SEQ ID NO: 99 - DNA, 23 bp, Homo sapiens
  misc_feature (21)..(21): n is a, c, g, or t
ggctctggagttgtggtgggngg

SEQ ID NO: 100 - DNA, 23 bp, Homo sapiens
  misc_feature (21)..(21): n is a, c, g, or t
catagctgatggtaccccccngg

SEQ ID NO: 101 - DNA, 23 bp, Homo sapiens
  misc_feature (21)..(21): n is a, c, g, or t
cggccagcactgtgccggcgngg

SEQ ID NO: 102 - DNA, 23 bp, Homo sapiens
  misc_feature (21)..(21): n is a, c, g, or t
caagaactcggccacttttcngg

SEQ ID NO: 103 - DNA, 23 bp, Homo sapiens
  misc_feature (21)..(21): n is a, c, g, or t
ggtgttcccgtggctcccctngg

SEQ ID NO: 104 - DNA, 23 bp, Homo sapiens
  misc_feature (21)..(21): n is a, c, g, or t
ccagcactgtgccggcgtggngg

SEQ ID NO: 105 - DNA, 23 bp, Homo sapiens
  misc_feature (21)..(21): n is a, c, g, or t
ggcaagggctggtgttcccgngg

SEQ ID NO: 106 - DNA, 23 bp, Homo sapiens
  misc_feature (21)..(21): n is a, c, g, or t
ggcgtggtggagttctacagngg

SEQ ID NO: 107 - DNA, 23 bp, Homo sapiens
  misc_feature (21)..(21): n is a, c, g, or t
ccaccacgccggcacagtgcngg

SEQ ID NO: 108 - DNA, 23 bp, Homo sapiens
  misc_feature (21)..(21): n is a, c, g, or t
ggagttctacagcggcagccngg

SEQ ID NO: 109 - DNA, 23 bp, Homo sapiens
  misc_feature (21)..(21): n is a, c, g, or t
gttctacagcggcagcctggngg

SEQ ID NO: 110 - DNA, 23 bp, Homo sapiens
  misc_feature (21)..(21): n is a, c, g, or t
accagcccttgccaatccaangg

SEQ ID NO: 111 - DNA, 23 bp, Homo sapiens
  misc_feature (21)..(21): n is a, c, g, or t
agttctacagcggcagcctgngg

SEQ ID NO: 112 - DNA, 23 bp, Homo sapiens
  misc_feature (21)..(21): n is a, c, g, or t
ccaggtcctgggtcttgtccngg

SEQ ID NO: 113 - DNA, 23 bp, Homo sapiens
  misc_feature (21)..(21): n is a, c, g, or t
tggtgttcccgtggctccccngg

SEQ ID NO: 114 - DNA, 23 bp, Homo sapiens
  misc_feature (21)..(21): n is a, c, g, or t
gagttctacagcggcagcctngg

SEQ ID NO: 115 - DNA, 23 bp, Homo sapiens
  misc_feature (21)..(21): n is a, c, g, or t
gaactcaagctgtacctcccngg

SEQ ID NO: 116 - DNA, 23 bp, Homo sapiens
  misc_feature (21)..(21): n is a, c, g, or t
aagaactcggccacttttctngg

SEQ ID NO: 117 - DNA, 23 bp, Homo sapiens
  misc_feature (21)..(21): n is a, c, g, or t
tccattggattggcaagggcngg

SEQ ID NO: 118 - DNA, 23 bp, Homo sapiens
  misc_feature (21)..(21): n is a, c, g, or t
ttctacagcggcagcctgggngg

SEQ ID NO: 119 - DNA, 23 bp, Homo sapiens
  misc_feature (21)..(21): n is a, c, g, or t
agaactcggccacttttctgngg

SEQ ID NO: 120 - DNA, 23 bp, Homo sapiens
  misc_feature (21)..(21): n is a, c, g, or t
gcttcaagaaggagccacacngg

SEQ ID NO: 121 - DNA, 23 bp, Homo sapiens
  misc_feature (21)..(21): n is a, c, g, or t
gatcttccattggattggcangg

SEQ ID NO: 122 - DNA, 23 bp, Homo sapiens
  misc_feature (21)..(21): n is a, c, g, or t
gctgtagaactccaccacgcngg

SEQ ID NO: 123 - DNA, 23 bp, Homo sapiens
  misc_feature (21)..(21): n is a, c, g, or t
gtcctgggcctcatagctgangg

SEQ ID NO: 124 - DNA, 23 bp, Homo sapiens
  misc_feature (21)..(21): n is a, c, g, or t
taccatcagctatgaggcccngg

SEQ ID NO: 125 - DNA, 23 bp, Homo sapiens
  misc_feature (21)..(21): n is a, c, g, or t
ggggggtaccatcagctatgngg

SEQ ID NO: 126 - DNA, 23 bp, Homo sapiens
  misc_feature (21)..(21): n is a, c, g, or t
cctgaagcaatgctccagggngg

SEQ ID NO: 127 - DNA, 23 bp, Homo sapiens
  misc_feature (21)..(21): n is a, c, g, or t
ttttcctgaagcaatgctccngg

SEQ ID NO: 128 - DNA, 23 bp, Homo sapiens
  misc_feature (21)..(21): n is a, c, g, or t
ctctggcagatgcttcaagangg

SEQ ID NO: 129 - DNA, 23 bp, Homo sapiens
  misc_feature (21)..(21): n is a, c, g, or t
agaggaagttctccaggtccngg

SEQ ID NO: 130 - DNA, 23 bp, Homo sapiens
  misc_feature (21)..(21): n is a, c, g, or t
tctggcggccagcactgtgcngg

SEQ ID NO: 131 - DNA, 23 bp, Homo sapiens
  misc_feature (21)..(21): n is a, c, g, or t
ttgagttctggatcttccatngg

SEQ ID NO: 132 - DNA, 23 bp, Homo sapiens
  misc_feature (21)..(21): n is a, c, g, or t
ttctggatcttccattggatngg

SEQ ID NO: 133 - DNA, 23 bp, Homo sapiens
  misc_feature (21)..(21): n is a, c, g, or t
atcttccattggattggcaangg

SEQ ID NO: 134 - DNA, 23 bp, Homo sapiens
  misc_feature (21)..(21): n is a, c, g, or t
tcaagaaggagccacactggngg

SEQ ID NO: 135 - DNA, 23 bp, Homo sapiens
  misc_feature (21)..(21): n is a, c, g, or t
gggaggtacagcttgagttcngg

SEQ ID NO: 136 - DNA, 23 bp, Homo sapiens
  misc_feature (21)..(21): n is a, c, g, or t
cccgtggctcccctgggtctngg

SEQ ID NO: 137 - DNA, 23 bp, Homo sapiens
  misc_feature (21)..(21): n is a, c, g, or t
ccaggacaagacccaggaccngg

SEQ ID NO: 138 - DNA, 23 bp, Homo sapiens
  misc_feature (21)..(21): n is a, c, g, or t
ctctgcaacaacctccagtgngg

SEQ ID NO: 139 - DNA, 23 bp, Homo sapiens
  misc_feature (21)..(21): n is a, c, g, or t
tgttgcagaggaagttctccngg

SEQ ID NO: 140 - DNA, 23 bp, Homo sapiens
  misc_feature (21)..(21): n is a, c, g, or t
caggtcctgggtcttgtcctngg

SEQ ID NO: 141 - DNA, 23 bp, Homo sapiens
  misc_feature (21)..(21): n is a, c, g, or t
tgaggcccaggacaagacccngg

SEQ ID NO: 142 - DNA, 23 bp, Homo sapiens
  misc_feature (21)..(21): n is a, c, g, or t
ctgtgccaccagctgcagccngg

SEQ ID NO: 143 - DNA, 23 bp, Homo sapiens
  misc_feature (21)..(21): n is a, c, g, or t
tgtgccaccagctgcagcctngg

SEQ ID NO: 144 - DNA, 23 bp, Homo sapiens
  misc_feature (21)..(21): n is a, c, g, or t
catctgccagagactgaggcngg

SEQ ID NO: 145 - DNA, 23 bp, Homo sapiens
  misc_feature (21)..(21): n is a, c, g, or t
ctgcagctggtggcacagtcngg

SEQ ID NO: 146 - DNA, 23 bp, Homo sapiens
  misc_feature (21)..(21): n is a, c, g, or t
cacactggaggttgttgcagngg

SEQ ID NO: 147 - DNA, 23 bp, Homo sapiens
  misc_feature (21)..(21): n is a, c, g, or t
cagctggtggcacagtctggngg

SEQ ID NO: 148 - DNA, 23 bp, Homo sapiens
  misc_feature (21)..(21): n is a, c, g, or t
agcaaaggagggcaagaactngg

SEQ ID NO: 149 - DNA, 23 bp, Homo sapiens
  misc_feature (21)..(21): n is a, c, g, or t
gaggaagttctccaggtcctngg

SEQ ID NO: 150 - DNA, 23 bp, Homo sapiens
  misc_feature (21)..(21): n is a, c, g, or t
gccaccagctgcagcctgggngg

SEQ ID NO: 151 - DNA, 23 bp, Homo sapiens
  misc_feature (21)..(21): n is a, c, g, or t
gcaggcagagcccaagacccngg

SEQ ID NO: 152 - DNA, 23 bp, Homo sapiens
  misc_feature (21)..(21): n is a, c, g, or t
caggcagagcccaagacccangg

SEQ ID NO: 153 - DNA, 23 bp, Homo sapiens
  misc_feature (21)..(21): n is a, c, g, or t
tcctcccaggctgcagctggngg

SEQ ID NO: 154 - DNA, 23 bp, Homo sapiens
  misc_feature (21)..(21): n is a, c, g, or t
gctctgcctgcctcagtctcngg

SEQ ID NO: 155 - DNA, 23 bp, Homo sapiens
  misc_feature (21)..(21): n is a, c, g, or t
cctccctggagcattgcttcngg

SEQ ID NO: 156 - DNA, 23 bp, Homo sapiens
  misc_feature (21)..(21): n is a, c, g, or t
tttcctgaagcaatgctccangg

SEQ ID NO: 157 - DNA, 23 bp, Homo sapiens
  misc_feature (21)..(21): n is a, c, g, or t
ccgtggctcccctgggtcttngg

SEQ ID NO: 158 - DNA, 23 bp, Homo sapiens
  misc_feature (21)..(21): n is a, c, g, or t
aaaatcaagccccagaaaagngg

SEQ ID NO: 159 - DNA, 23 bp, Homo sapiens
  misc_feature (21)..(21): n is a, c, g, or t
gaagcatctgccagagactgngg

SEQ ID NO: 160 - DNA, 23 bp, Homo sapiens
  misc_feature (21)..(21): n is a, c, g, or t
ccaagacccaggggagccacngg

SEQ ID NO: 161 - DNA, 23 bp, Homo sapiens
  misc_feature (21)..(21): n is a, c, g, or t
aggcagagcccaagacccagngg

SEQ ID NO: 162 - DNA, 23 bp, Homo sapiens
  misc_feature (21)..(21): n is a, c, g, or t
cccaagacccaggggagccangg

SEQ ID NO: 163 - DNA, 23 bp, Homo sapiens
  misc_feature (21)..(21): n is a, c, g, or t
tactcaggttcaggagacgcngg

SEQ ID NO: 164 - DNA, 23 bp, Homo sapiens
  misc_feature (21)..(21): n is a, c, g, or t
tcaggagacgccgggcctccngg

SEQ ID NO: 165 - DNA, 23 bp, Homo sapiens
  misc_feature (21)..(21): n is a, c, g, or t
actcaggttcaggagacgccngg

SEQ ID NO: 166 - DNA, 23 bp, Homo sapiens
  misc_feature (21)..(21): n is a, c, g, or t
cagtgtctctactcaggttcngg

SEQ ID NO: 167 - DNA, 23 bp, Homo sapiens
  misc_feature (21)..(21): n is a, c, g, or t
atgctcccagggctgcgtgcngg

SEQ ID NO: 168 - DNA, 23 bp, Homo sapiens
  misc_feature (21)..(21): n is a, c, g, or t
gagacgccgggcctcctggangg

SEQ ID NO: 169 - DNA, 23 bp, Homo sapiens
  misc_feature (21)..(21): n is a, c, g, or t
cagcagcagtgtctctactcngg

SEQ ID NO: 170 - DNA, 23 bp, Homo sapiens
  misc_feature (21)..(21): n is a, c, g, or t
gatggcattcacatgctcccngg

SEQ ID NO: 171 - DNA, 23 bp, Homo sapiens
  misc_feature (21)..(21): n is a, c, g, or t
ggagcatgtgaatgccatccngg

SEQ ID NO: 172 - DNA, 23 bp, Homo sapiens
  misc_feature (21)..(21): n is a, c, g, or t
tagagacactgctgctgagangg

SEQ ID NO: 173 - DNA, 23 bp, Homo sapiens
  misc_feature (21)..(21): n is a, c, g, or t
gcatgtgaatgccatccaggngg

SEQ ID NO: 174 - DNA, 23 bp, Homo sapiens
  misc_feature (21)..(21): n is a, c, g, or t
atggcattcacatgctcccangg

SEQ ID NO: 175 - DNA, 23 bp, Homo sapiens
  misc_feature (21)..(21): n is a, c, g, or t
tgaatgccatccaggaggccngg

SEQ ID NO: 176 - DNA, 23 bp, Homo sapiens
  misc_feature (21)..(21): n is a, c, g, or t
tgctcccagggctgcgtgctngg

SEQ ID NO: 177 - DNA, 23 bp, Homo sapiens
  misc_feature (21)..(21): n is a, c, g, or t
cagccccagcacgcagccctngg

SEQ ID NO: 178 - DNA, 23 bp, Homo sapiens
  misc_feature (21)..(21): n is a, c, g, or t
gctcccagggctgcgtgctgngg

SEQ ID NO: 179 - DNA, 23 bp, Homo sapiens
  misc_feature (21)..(21): n is a, c, g, or t
ccttggatttcagcggcacangg

SEQ ID NO: 180 - DNA, 23 bp, Homo sapiens
  misc_feature (21)..(21): n is a, c, g, or t
ccttgtgccgctgaaatccangg

SEQ ID NO: 181 - DNA, 23 bp, Homo sapiens
  misc_feature (21)..(21): n is a, c, g, or t
tgaacctggctaccaggaccngg

SEQ ID NO: 182 - DNA, 23 bp, Homo sapiens
  misc_feature (21)..(21): n is a, c, g, or t
agggccaggtcctggtagccngg

SEQ ID NO: 183 - DNA, 23 bp, Homo sapiens
  misc_feature (21)..(21): n is a, c, g, or t
ctcagctgaacctggctaccngg

SEQ ID NO: 184 - DNA, 23 bp, Homo sapiens
  misc_feature (21)..(21): n is a, c, g, or t
agaaaaaacaggagagtgcangg

SEQ ID NO: 185 - DNA, 23 bp, Homo sapiens
  misc_feature (21)..(21): n is a, c, g, or t
gcacaaggctcagctgaaccngg

SEQ ID NO: 186 - DNA, 23 bp, Homo sapiens
  misc_feature (21)..(21): n is a, c, g, or t
tggcttgccttggatttcagngg

SEQ ID NO: 187 - DNA, 23 bp, Homo sapiens
  misc_feature (21)..(21): n is a, c, g, or t
aaacaggagagtgcagggccngg

SEQ ID NO: 188 - DNA, 23 bp, Homo sapiens
  misc_feature (21)..(21): n is a, c, g, or t
gagagtgcagggccaggtccngg

SEQ ID NO: 189 - DNA, 23 bp, Homo sapiens
  misc_feature (21)..(21): n is a, c, g, or t
ggatgaagagaagaaaaaacngg

SEQ ID NO: 190 - DNA, 23 bp, Homo sapiens
  misc_feature (21)..(21): n is a, c, g, or t
aagaaaaaacaggagagtgcngg

SEQ ID NO: 191 - DNA, 23 bp, Homo sapiens
  misc_feature (21)..(21): n is a, c, g, or t
ccgggtgacagtgcttcggcngg

SEQ ID NO: 192 - DNA, 23 bp, Homo sapiens
  misc_feature (21)..(21): n is a, c, g, or t
acacaaagctggcgatgcctngg

SEQ ID NO: 193 - DNA, 23 bp, Homo sapiens
  misc_feature (21)..(21): n is a, c, g, or t
ccctcagtccttggatagtgngg

SEQ ID NO: 194 - DNA, 23 bp, Homo sapiens
  misc_feature (21)..(21): n is a, c, g, or t
gtgcggcaacctacatgatgngg

SEQ ID NO: 195 - DNA, 23 bp, Homo sapiens
  misc_feature (21)..(21): n is a, c, g, or t
ctgtgcggcaacctacatgangg

SEQ ID NO: 196 - DNA, 23 bp, Homo sapiens
  misc_feature (21)..(21): n is a, c, g, or t
ggcccagcctgctgtggtacngg

SEQ ID NO: 197 - DNA, 23 bp, Homo sapiens
  misc_feature (21)..(21): n is a, c, g, or t
gttcacttgatttccactggngg

SEQ ID NO: 198 - DNA, 23 bp, Homo sapiens
  misc_feature (21)..(21): n is a, c, g, or t
caactcattccccatcatgtngg

SEQ ID NO: 199 - DNA, 23 bp, Homo sapiens
  misc_feature (21)..(21): n is a, c, g, or t
ccgcacagacttcagtcaccngg

SEQ ID NO: 200 - DNA, 23 bp, Homo sapiens
  misc_feature (21)..(21): n is a, c, g, or t
tgtgcggcaacctacatgatngg

SEQ ID NO: 201 - DNA, 23 bp, Homo sapiens
  misc_feature (21)..(21): n is a, c, g, or t
cctcactatccaaggactgangg

SEQ ID NO: 202 - DNA, 23 bp, Homo sapiens
  misc_feature (21)..(21): n is a, c, g, or t
cggacctcagtggctttgccngg

SEQ ID NO: 203 - DNA, 23 bp, Homo sapiens
  misc_feature (21)..(21): n is a, c, g, or t
gaggttcacttgatttccacngg

SEQ ID NO: 204 - DNA, 23 bp, Homo sapiens
  misc_feature (21)..(21): n is a, c, g, or t
ccaggtgactgaagtctgtgngg

SEQ ID NO: 205 - DNA, 23 bp, Homo sapiens
  misc_feature (21)..(21): n is a, c, g, or t
actggaggtgcccgtgcagangg

SEQ ID NO: 206 - DNA, 23 bp, Homo sapiens
  misc_feature (21)..(21): n is a, c, g, or t
caagtgaacctcactatccangg

SEQ ID NO: 207 - DNA, 23 bp, Homo sapiens
  misc_feature (21)..(21): n is a, c, g, or t
gtggtactggccagcagccgngg

SEQ ID NO: 208 - DNA, 23 bp, Homo sapiens
  misc_feature (21)..(21): n is a, c, g, or t
aggtccgggtgacagtgcttngg

SEQ ID NO: 209 - DNA, 23 bp, Homo sapiens
  misc_feature (21)..(21): n is a, c, g, or t
atctgcacgggcacctccagngg

SEQ ID NO: 210 - DNA, 23 bp, Homo sapiens
  misc_feature (21)..(21): n is a, c, g, or t
ccgtgcagatggaatcatctngg

SEQ ID NO: 211 - DNA, 23 bp, Homo sapiens
  misc_feature (21)..(21): n is a, c, g, or t
ctagatgattccatctgcacngg

SEQ ID NO: 212 - DNA, 23 bp, Homo sapiens
  misc_feature (21)..(21): n is a, c, g, or t
acctcactatccaaggactgngg

SEQ ID NO: 213 - DNA, 23 bp, Homo sapiens
  misc_feature (21)..(21): n is a, c, g, or t
cctgccgaagcactgtcaccngg

SEQ ID NO: 214 - DNA, 23 bp, Homo sapiens
  misc_feature (21)..(21): n is a, c, g, or t
tggccagtaccacagcaggcngg

SEQ ID NO: 215 - DNA, 23 bp, Homo sapiens
  misc_feature (21)..(21): n is a, c, g, or t
atctccaggcaaagccactgngg

SEQ ID NO: 216 - DNA, 23 bp, Homo sapiens
  misc_feature (21)..(21): n is a, c, g, or t
gcacgtggcccagcctgctgngg

SEQ ID NO: 217 - DNA, 23 bp, Homo sapiens
  misc_feature (21)..(21): n is a, c, g, or t
gtgtgtgagtatgcatctccngg

SEQ ID NO: 218 - DNA, 23 bp, Homo sapiens
  misc_feature (21)..(21): n is a, c, g, or t
cactgtcacccggacctcagngg

SEQ ID NO: 219 - DNA, 23 bp, Homo sapiens
  misc_feature (21)..(21): n is a, c, g, or t
gctggcgatgcctcggctgcngg

SEQ ID NO: 220 - DNA, 23 bp, Homo sapiens
  misc_feature (21)..(21): n is a, c, g, or t
ctgctggccagtaccacagcngg

SEQ ID NO: 221 - DNA, 23 bp, Homo sapiens
  misc_feature (21)..(21): n is a, c, g, or t
aggcaaagccactgaggtccngg

SEQ ID NO: 222 - DNA, 23 bp, Homo sapiens
  misc_feature (21)..(21): n is a, c, g, or t
gcagatggaatcatctaggangg

SEQ ID NO: 223 - DNA, 23 bp, Homo sapiens
  misc_feature (21)..(21): n is a, c, g, or t
cctagatgattccatctgcangg

SEQ ID NO: 224 - DNA, 23 bp, Homo sapiens
  misc_feature (21)..(21): n is a, c, g, or t
ggccagtaccacagcaggctngg

SEQ ID NO: 225 - DNA, 23 bp, Homo sapiens
  misc_feature (21)..(21): n is a, c, g, or t
tgcatactcacacacaaagcngg

SEQ ID NO: 226 - DNA, 23 bp, Homo sapiens
  misc_feature (21)..(21): n is a, c, g, or t
gcttcggcaggctgacagccngg

SEQ ID NO: 227 - DNA, 23 bp, Homo sapiens
  misc_feature (21)..(21): n is a, c, g, or t
caggcaaagccactgaggtcngg

SEQ ID NO: 228 - DNA, 23 bp, Homo sapiens
  misc_feature (21)..(21): n is a, c, g, or t
tgtagcaccgcccagacgacngg

SEQ ID NO: 229 - DNA, 23 bp, Homo sapiens
  misc_feature (21)..(21): n is a, c, g, or t
ggcgccctggccagtcgtctngg

SEQ ID NO: 230 - DNA, 23 bp, Homo sapiens
  misc_feature (21)..(21): n is a, c, g, or t
cgtctgggcggtgctacaacngg

SEQ ID NO: 231 - DNA, 23 bp, Homo sapiens
  misc_feature (21)..(21): n is a, c, g, or t
aggcgccctggccagtcgtcngg

SEQ ID NO: 232 - DNA, 23 bp, Homo sapiens
  misc_feature (21)..(21): n is a, c, g, or t
caccgcccagacgactggccngg

SEQ ID NO: 233 - DNA, 23 bp, Homo sapiens
  misc_feature (21)..(21): n is a, c, g, or t
accgcccagacgactggccangg

SEQ ID NO: 234 - DNA, 23 bp, Homo sapiens
  misc_feature (21)..(21): n is a, c, g, or t
gggcggtgctacaactgggcngg

SEQ ID NO: 235 - DNA, 23 bp, Homo sapiens
  misc_feature (21)..(21): n is a, c, g, or t
gtctgggcggtgctacaactngg

SEQ ID NO: 236 - DNA, 23 bp, Homo sapiens
  misc_feature (21)..(21): n is a, c, g, or t
cgactggccagggcgcctgtngg

SEQ ID NO: 237 - DNA, 23 bp, Homo sapiens
  misc_feature (21)..(21): n is a, c, g, or t
cggtgctacaactgggctggngg

SEQ ID NO: 238 - DNA, 23 bp, Homo sapiens
  misc_feature (21)..(21): n is a, c, g, or t
tggcggccaggatggttcttngg

SEQ ID NO: 239 - DNA, 23 bp, Homo sapiens
  misc_feature (21)..(21): n is a, c, g, or t
acgactggccagggcgcctgngg

SEQ ID NO: 240 - DNA, 23 bp, Homo sapiens
  misc_feature (21)..(21): n is a, c, g, or t
ctacaactgggctggcggccngg

SEQ ID NO: 241 - DNA, 23 bp, Homo sapiens
  misc_feature (21)..(21): n is a, c, g, or t
gccctggccagtcgtctgggngg

SEQ ID NO: 242 - DNA, 23 bp, Homo sapiens
  misc_feature (21)..(21): n is a, c, g, or t
tgcagatcccacaggcgcccngg

SEQ ID NO: 243 - DNA, 23 bp, Homo sapiens
  misc_feature (21)..(21): n is a, c, g, or t
aactgggctggcggccaggangg

SEQ ID NO: 244 - DNA, 23 bp, Homo sapiens
  misc_feature (21)..(21): n is a, c, g, or t
cggagagcttcgtgctaaacngg

SEQ ID NO: 245 - DNA, 23 bp, Homo sapiens
  misc_feature (21)..(21): n is a, c, g, or t
gcgtgacttccacatgagcgngg

SEQ ID NO: 246 - DNA, 23 bp, Homo sapiens
  misc_feature (21)..(21): n is a, c, g, or t
atgtggaagtcacgcccgttngg

SEQ ID NO: 247 - DNA, 23 bp, Homo sapiens
  misc_feature (21)..(21): n is a, c, g, or t
gccctgctcgtggtgaccgangg

SEQ ID NO: 248 - DNA, 23 bp, Homo sapiens
  misc_feature (21)..(21): n is a, c, g, or t
cacgaagctctccgatgtgtngg

SEQ ID NO: 249 - DNA, 23 bp, Homo sapiens
  misc_feature (21)..(21): n is a, c, g, or t
cctgctcgtggtgaccgaagngg

SEQ ID NO: 250 - DNA, 23 bp, Homo sapiens
  misc_feature (21)..(21): n is a, c, g, or t
tgacacggaagcggcagtccngg

SEQ ID NO: 251 - DNA, 23 bp, Homo sapiens
  misc_feature (21)..(21): n is a, c, g, or t
ccccttcggtcaccacgagcngg

SEQ ID NO: 252 - DNA, 23 bp, Homo sapiens
  misc_feature (21)..(21): n is a, c, g, or t
cagcaaccagacggacaagcngg

SEQ ID NO: 253 - DNA, 23 bp, Homo sapiens
  misc_feature (21)..(21): n is a, c, g, or t
gcagttgtgtgacacggaagngg

SEQ ID NO: 254 - DNA, 23 bp, Homo sapiens
  misc_feature (21)..(21): n is a, c, g, or t
cccttcggtcaccacgagcangg

SEQ ID NO: 255 - DNA, 23 bp, Homo sapiens
  misc_feature (21)..(21): n is a, c, g, or t
ccgggctggctgcggtcctcngg

SEQ ID NO: 256 - DNA, 23 bp, Homo sapiens
  misc_feature (21)..(21): n is a, c, g, or t
aggcggccagcttgtccgtcngg

SEQ ID NO: 257 - DNA, 23 bp, Homo sapiens
  misc_feature (21)..(21): n is a, c, g, or t
cagcttgtccgtctggttgcngg

SEQ ID NO: 258 - DNA, 23 bp, Homo sapiens
  misc_feature (21)..(21): n is a, c, g, or t
cggtcaccacgagcagggctngg

SEQ ID NO: 259 - DNA, 23 bp, Homo sapiens
  misc_feature (21)..(21): n is a, c, g, or t
gtgtcacacaactgcccaacngg

SEQ ID NO: 260 - DNA, 23 bp, Homo sapiens
  misc_feature (21)..(21): n is a, c, g, or t
ctgcagcttctccaacacatngg

SEQ ID NO: 261 - DNA, 23 bp, Homo sapiens
  misc_feature (21)..(21): n is a, c, g, or t
caagctggccgccttccccgngg

SEQ ID NO: 262 - DNA, 23 bp, Homo sapiens
  misc_feature (21)..(21): n is a, c, g, or t
cgtgtcacacaactgcccaangg

SEQ ID NO: 263 - DNA, 23 bp, Homo sapiens
  misc_feature (21)..(21): n is a, c, g, or t
cgttgggcagttgtgtgacangg

SEQ ID NO: 264 - DNA, 23 bp, Homo sapiens
  misc_feature (21)..(21): n is a, c, g, or t
gcttgtccgtctggttgctgngg

SEQ ID NO: 265 - DNA, 23 bp, Homo sapiens
  misc_feature (21)..(21): n is a, c, g, or t
cggaagcggcagtcctggccngg

SEQ ID NO: 266 - DNA, 23 bp, Homo sapiens
  misc_feature (21)..(21): n is a, c, g, or t
cgatgtgttggagaagctgcngg

SEQ ID NO: 267 - DNA, 23 bp, Homo sapiens
  misc_feature (21)..(21): n is a, c, g, or t
catgtggaagtcacgcccgtngg

SEQ ID NO: 268 - DNA, 23 bp, Homo sapiens
  misc_feature (21)..(21): n is a, c, g, or t
ccctgctcgtggtgaccgaangg

SEQ ID NO: 269 - DNA, 23 bp, Homo sapiens
  misc_feature (21)..(21): n is a, c, g, or t
cgggctggctgcggtcctcgngg

SEQ ID NO: 270 - DNA, 23 bp, Homo sapiens
  misc_feature (21)..(21): n is a, c, g, or t
agcttgtccgtctggttgctngg

SEQ ID NO: 271 - DNA, 23 bp, Homo sapiens
  misc_feature (21)..(21): n is a, c, g, or t
gaaggtggcgttgtccccttngg

SEQ ID NO: 272 - DNA, 23 bp, Homo sapiens
  misc_feature (21)..(21): n is a, c, g, or t
acttccacatgagcgtggtcngg

SEQ ID NO: 273 - DNA, 23 bp, Homo sapiens
  misc_feature (21)..(21): n is a, c, g, or t
gccgggctggctgcggtcctngg

SEQ ID NO: 274 - DNA, 23 bp, Homo sapiens
  misc_feature (21)..(21): n is a, c, g, or t
tcggtcaccacgagcagggcngg

SEQ ID NO: 275 - DNA, 23 bp, Homo sapiens
  misc_feature (21)..(21): n is a, c, g, or t
tctggttgctggggctcatgngg

SEQ ID NO: 276 - DNA, 23 bp, Homo sapiens
  misc_feature (21)..(21): n is a, c, g, or t
acggaagcggcagtcctggcngg

SEQ ID NO: 277 - DNA, 23 bp, Homo sapiens
  misc_feature (21)..(21): n is a, c, g, or t
cccgaggaccgcagccagccngg

SEQ ID NO: 278 - DNA, 23 bp, Homo sapiens
  misc_feature (21)..(21): n is a, c, g, or t
ctggctgcggtcctcggggangg

SEQ ID NO: 279 - DNA, 23 bp, Homo sapiens
  misc_feature (21)..(21): n is a, c, g, or t
catgagccccagcaaccagangg

SEQ ID NO: 280 - DNA, 23 bp, Homo sapiens
  misc_feature (21)..(21): n is a, c, g, or t
agtcctggccgggctggctgngg

SEQ ID NO: 281 - DNA, 23 bp, Homo sapiens
  misc_feature (21)..(21): n is a, c, g, or t
gggggttccagggcctgtctngg

SEQ ID NO: 282 - DNA, 23 bp, Homo sapiens
  misc_feature (21)..(21): n is a, c, g, or t
ggtcaccacgagcagggctgngg

SEQ ID NO: 283 - DNA, 23 bp, Homo sapiens
  misc_feature (21)..(21): n is a, c, g, or t
gctgcggtcctcggggaaggngg

SEQ ID NO: 284 - DNA, 23 bp, Homo sapiens
  misc_feature (21)..(21): n is a, c, g, or t
ggaccgcagccagcccggccngg

SEQ ID NO: 285 - DNA, 23 bp, Homo sapiens
  misc_feature (21)..(21): n is a, c, g, or t
gagaaggtgggggggttccangg

SEQ ID NO: 286 - DNA, 23 bp, Homo sapiens
  misc_feature (21)..(21): n is a, c, g, or t
ggagaaggtgggggggttccngg

SEQ ID NO: 287 - DNA, 23 bp, Homo sapiens
  misc_feature (21)..(21): n is a, c, g, or t
agcggcagtcctggccgggcngg

SEQ ID NO: 288 - DNA, 23 bp, Homo sapiens
  misc_feature (21)..(21): n is a, c, g, or t
ggggttccagggcctgtctgngg

SEQ ID NO: 289 - DNA, 23 bp, Homo sapiens
  misc_feature (21)..(21): n is a, c, g, or t
cttctccccagccctgctcgngg

SEQ ID NO: 290 - DNA, 23 bp, Homo sapiens
  misc_feature (21)..(21): n is a, c, g, or t
gttggagaagctgcaggtgangg

SEQ ID NO: 291 - DNA, 23 bp, Homo sapiens
  misc_feature (21)..(21): n is a, c, g, or t
ggggggttccagggcctgtcngg

SEQ ID NO: 292 - DNA, 23 bp, Homo sapiens
  misc_feature (21)..(21): n is a, c, g, or t
ggagaagctgcaggtgaaggngg

SEQ ID NO: 293 - DNA, 23 bp, Homo sapiens
  misc_feature (21)..(21): n is a, c, g, or t
cacgagcagggctggggagangg

SEQ ID NO: 294 - DNA, 23 bp, Homo sapiens
  misc_feature (21)..(21): n is a, c, g, or t
gcagggctggggagaaggtgngg

SEQ ID NO: 295 - DNA, 23 bp, Homo sapiens
  misc_feature (21)..(21): n is a, c, g, or t
cagggctggggagaaggtggngg

SEQ ID NO: 296 - DNA, 23 bp, Homo sapiens
  misc_feature (21)..(21): n is a, c, g, or t
gagcagggctggggagaaggngg

SEQ ID NO: 297 - DNA, 23 bp, Homo sapiens
  misc_feature (21)..(21): n is a, c, g, or t
agcagggctggggagaaggtngg

SEQ ID NO: 298 - DNA, 23 bp, Homo sapiens
  misc_feature (21)..(21): n is a, c, g, or t
agggctggggagaaggtgggngg

SEQ ID NO: 299 - DNA, 23 bp, Homo sapiens
  misc_feature (21)..(21): n is a, c, g, or t
gggctggggagaaggtggggngg

SEQ ID NO: 300 - DNA, 23 bp, Homo sapiens
  misc_feature (21)..(21): n is a, c, g, or t
agaagtggaatacagagcggngg

SEQ ID NO: 301 - DNA, 23 bp, Homo sapiens
  misc_feature (21)..(21): n is a, c, g, or t
aatgtggcaacgtggtgctcngg

SEQ ID NO: 302 - DNA, 23 bp, Homo sapiens
  misc_feature (21)..(21): n is a, c, g, or t
ctaaatggggatttccgcaangg

SEQ ID NO: 303 - DNA, 23 bp, Homo sapiens
  misc_feature (21)..(21): n is a, c, g, or t
catccagatactggctaaatngg

SEQ ID NO: 304 - DNA, 23 bp, Homo sapiens
  misc_feature (21)..(21): n is a, c, g, or t
cagacgggcacgaggttcccngg

SEQ ID NO: 305 - DNA, 23 bp, Homo sapiens
  misc_feature (21)..(21): n is a, c, g, or t
gcggctggggtgtagaagcangg

SEQ ID NO: 306 - DNA, 23 bp, Homo sapiens
  misc_feature (21)..(21): n is a, c, g, or t
gaacctcgtgcccgtctgctngg

SEQ ID NO: 307 - DNA, 23 bp, Homo sapiens
  misc_feature (21)..(21): n is a, c, g, or t
gacgggcacgaggttccctgngg

SEQ ID NO: 308 - DNA, 23 bp, Homo sapiens
  misc_feature (21)..(21): n is a, c, g, or t
atccccatttagccagtatcngg

SEQ ID NO: 309 - DNA, 23 bp, Homo sapiens
  misc_feature (21)..(21): n is a, c, g, or t
gtggaatacagagcggaggtngg

SEQ ID NO: 310 - DNA, 23 bp, Homo sapiens
  misc_feature (21)..(21): n is a, c, g, or t
agacgggcacgaggttccctngg

SEQ ID NO: 311 - DNA, 23 bp, Homo sapiens
  misc_feature (21)..(21): n is a, c, g, or t
ggaacctcgtgcccgtctgcngg

SEQ ID NO: 312 - DNA, 23 bp, Homo sapiens
  misc_feature (21)..(21): n is a, c, g, or t
gagtcacattctctatggtcngg

SEQ ID NO: 313 - DNA, 23 bp, Homo sapiens
  misc_feature (21)..(21): n is a, c, g, or t
atgtgactctagcagacagtngg

SEQ ID NO: 314 - DNA, 23 bp, Homo sapiens
  misc_feature (21)..(21): n is a, c, g, or t
ttttcatcattcattatgccngg

SEQ ID NO: 315 - DNA, 23 bp, Homo sapiens
  misc_feature (21)..(21): n is a, c, g, or t
aatgtgactctagcagacagngg

SEQ ID NO: 316 - DNA, 23 bp, Homo sapiens
  misc_feature (21)..(21): n is a, c, g, or t
atccagatactggctaaatgngg

SEQ ID NO: 317 - DNA, 23 bp, Homo sapiens
  misc_feature (21)..(21): n is a, c, g, or t
tgctgccggatccaaatcccngg

SEQ ID NO: 318 - DNA, 23 bp, Homo sapiens
  misc_feature (21)..(21): n is a, c, g, or t
tctacaccccagccgccccangg

SEQ ID NO: 319 - DNA, 23 bp, Homo sapiens
  misc_feature (21)..(21): n is a, c, g, or t
ttatgcctgggatttggatcngg

SEQ ID NO: 320 - DNA, 23 bp, Homo sapiens
  misc_feature (21)..(21): n is a, c, g, or t
cgctctgtattccacttctgngg

SEQ ID NO: 321 - DNA, 23 bp, Homo sapiens
  misc_feature (21)..(21): n is a, c, g, or t
gaggttccctggggcggctgngg

SEQ ID NO: 322 - DNA, 23 bp, Homo sapiens
  misc_feature (21)..(21): n is a, c, g, or t
tgccccagcagacgggcacgngg

SEQ ID NO: 323 - DNA, 23 bp, Homo sapiens
  misc_feature (21)..(21): n is a, c, g, or t
acagtgggatctactgctgcngg

SEQ ID NO: 324 - DNA, 23 bp, Homo sapiens
  misc_feature (21)..(21): n is a, c, g, or t
tgtgtttgaatgtggcaacgngg

SEQ ID NO: 325 - DNA, 23 bp, Homo sapiens
  misc_feature (21)..(21): n is a, c, g, or t
tgaaaaatttaacctgaagtngg

SEQ ID NO: 326 - DNA, 23 bp, Homo sapiens
  misc_feature (21)..(21): n is a, c, g, or t
acatccagatactggctaaangg

SEQ ID NO: 327 - DNA, 23 bp, Homo sapiens
  misc_feature (21)..(21): n is a, c, g, or t
atgaaagggatgtgaattatngg

SEQ ID NO: 328 - DNA, 23 bp, Homo sapiens
  misc_feature (21)..(21): n is a, c, g, or t
tggtgctcaggactgatgaangg

SEQ ID NO: 329 - DNA, 23 bp, Homo sapiens
  misc_feature (21)..(21): n is a, c, g, or t
ggtgtagaagcagggcagatngg

SEQ ID NO: 330 - DNA, 23 bp, Homo sapiens
  misc_feature (21)..(21): n is a, c, g, or t
acgttgccacattcaaacacngg

SEQ ID NO: 331 - DNA, 23 bp, Homo sapiens
  misc_feature (21)..(21): n is a, c, g, or t
acgaggttccctggggcggcngg

SEQ ID NO: 332 - DNA, 23 bp, Homo sapiens
  misc_feature (21)..(21): n is a, c, g, or t
gcctgtcctgtgtttgaatgngg

SEQ ID NO: 333 - DNA, 23 bp, Homo sapiens
  misc_feature (21)..(21): n is a, c, g, or t
gtgcccgtctgctggggcaangg

SEQ ID NO: 334 - DNA, 23 bp, Homo sapiens
  misc_feature (21)..(21): n is a, c, g, or t
aacctcgtgcccgtctgctgngg

SEQ ID NO: 335 - DNA, 23 bp, Homo sapiens
  misc_feature (21)..(21): n is a, c, g, or t
ggcggctggggtgtagaagcngg

SEQ ID NO: 336 - DNA, 23 bp, Homo sapiens
  misc_feature (21)..(21): n is a, c, g, or t
agtcacattctctatggtcangg

SEQ ID NO: 337 - DNA, 23 bp, Homo sapiens
  misc_feature (21)..(21): n is a, c, g, or t
ctggtttgatgaccaacttcngg

SEQ ID NO: 338 - DNA, 23 bp, Homo sapiens
  misc_feature (21)..(21): n is a, c, g, or t
cattcattatgcctgggattngg

SEQ ID NO: 339 - DNA, 23 bp, Homo sapiens
  misc_feature (21)..(21): n is a, c, g, or t
tgctagagtcacattctctangg

SEQ ID NO: 340 - DNA, 23 bp, Homo sapiens
  misc_feature (21)..(21): n is a, c, g, or t
gggcacgaggttccctggggngg

SEQ ID NO: 341 - DNA, 23 bp, Homo sapiens
  misc_feature (21)..(21): n is a, c, g, or t
ggctcctttgccccagcagangg

SEQ ID NO: 342 - DNA, 23 bp, Homo sapiens
  misc_feature (21)..(21): n is a, c, g, or t
attattggacatccagatacngg

SEQ ID NO: 343 - DNA, 23 bp, Homo sapiens
  misc_feature (21)..(21): n is a, c, g, or t
tttcatcattcattatgcctngg

SEQ ID NO: 344 - DNA, 23 bp, Homo sapiens
  misc_feature (21)..(21): n is a, c, g, or t
ttctacaccccagccgccccngg

SEQ ID NO: 345 - DNA, 23 bp, Homo sapiens
  misc_feature (21)..(21): n is a, c, g, or t
tcagggacacatctcctttgngg

SEQ ID NO: 346 - DNA, 23 bp, Homo sapiens
  misc_feature (21)..(21): n is a, c, g, or t
gctcctttgccccagcagacngg

SEQ ID NO: 347 - DNA, 23 bp, Homo sapiens
  misc_feature (21)..(21): n is a, c, g, or t
ctcagaagtggaatacagagngg

SEQ ID NO: 348 - DNA, 23 bp, Homo sapiens
  misc_feature (21)..(21): n is a, c, g, or t
cgaggttccctggggcggctngg

SEQ ID NO: 349 - DNA, 23 bp, Homo sapiens
  misc_feature (21)..(21): n is a, c, g, or t
gccacattcaaacacaggacngg

SEQ ID NO: 350 - DNA, 23 bp, Homo sapiens
  misc_feature (21)..(21): n is a, c, g, or t
ggtgctcaggactgatgaaangg

SEQ ID NO: 351 - DNA, 23 bp, Homo sapiens
  misc_feature (21)..(21): n is a, c, g, or t
aggtcacccctgcaccgactngg

SEQ ID NO: 352 - DNA, 23 bp, Homo sapiens
  misc_feature (21)..(21): n is a, c, g, or t
ctctctgccgagtcggtgcangg

SEQ ID NO: 353 - DNA, 23 bp, Homo sapiens
  misc_feature (21)..(21): n is a, c, g, or t
tctctctgccgagtcggtgcngg

SEQ ID NO: 354 - DNA, 23 bp, Homo sapiens
  misc_feature (21)..(21): n is a, c, g, or t
ccaaggatgcttaccaccagngg

SEQ ID NO: 355 - DNA, 23 bp, Homo sapiens
  misc_feature (21)..(21): n is a, c, g, or t
tctctgccgagtcggtgcagngg

SEQ ID NO: 356 - DNA, 23 bp, Homo sapiens
  misc_feature (21)..(21): n is a, c, g, or t
cccctggtggtaagcatcctngg

SEQ ID NO: 357 - DNA, 23 bp, Homo sapiens
  misc_feature (21)..(21): n is a, c, g, or t
tccaaggatgcttaccaccangg

SEQ ID NO: 358 - DNA, 23 bp, Homo sapiens
  misc_feature (21)..(21): n is a, c, g, or t
ggtggtaagcatccttggaangg

SEQ ID NO: 359 - DNA, 23 bp, Homo sapiens
  misc_feature (21)..(21): n is a, c, g, or t
gtgaagtctctctgccgagtngg

SEQ ID NO: 360 - DNA, 23 bp, Homo sapiens
  misc_feature (21)..(21): n is a, c, g, or t
atgcttaccaccaggggacangg

SEQ ID NO: 361 - DNA, 23 bp, Homo sapiens
  misc_feature (21)..(21): n is a, c, g, or t
ttccaaggatgcttaccaccngg

SEQ ID NO: 362 - DNA, 23 bp, Homo sapiens
  misc_feature (21)..(21): n is a, c, g, or t
agtcggtgcaggggtgacctngg

SEQ ID NO: 363 - DNA, 23 bp, Homo sapiens
  misc_feature (21)..(21): n is a, c, g, or t
acttcactgcagcctttccangg

SEQ ID NO: 364 - DNA, 23 bp, Homo sapiens
  misc_feature (21)..(21): n is a, c, g, or t
caaagagattacgaatgcctngg

SEQ ID NO: 365 - DNA, 23 bp, Homo sapiens
  misc_feature (21)..(21): n is a, c, g, or t
caaggcattcgtaatctcttngg

SEQ ID NO: 366 - DNA, 23 bp, Homo sapiens
  misc_feature (21)..(21): n is a, c, g, or t
cttgtagatatcctgatcatngg

SEQ ID NO: 367 - DNA, 23 bp, Homo sapiens
  misc_feature (21)..(21): n is a, c, g, or t
cttgggtcaggacatcaactngg

SEQ ID NO: 368 - DNA, 23 bp, Homo sapiens
  misc_feature (21)..(21): n is a, c, g, or t
cgatgatcaggatatctacangg

SEQ ID NO: 369 - DNA, 23 bp, Homo sapiens
  misc_feature (21)..(21): n is a, c, g, or t
ttacgaatgccttggaaaccngg

SEQ ID NO: 370 - DNA, 23 bp, Homo sapiens
  misc_feature (21)..(21): n is a, c, g, or t
tacgaatgccttggaaacctngg

SEQ ID NO: 371 - DNA, 23 bp, Homo sapiens
  misc_feature (21)..(21): n is a, c, g, or t
acgaatgccttggaaacctgngg

SEQ ID NO: 372 - DNA, 23 bp, Homo sapiens
  misc_feature (21)..(21): n is a, c, g, or t
tgatattgacgatataaaatngg

SEQ ID NO: 373 - DNA, 23 bp, Homo sapiens
  misc_feature (21)..(21): n is a, c, g, or t
atgatattgacgatataaaangg

SEQ ID NO: 374 - DNA, 23 bp, Homo sapiens
  misc_feature (21)..(21): n is a, c, g, or t
gcatctgaagaccgatgatcngg

SEQ ID NO: 375 - DNA, 23 bp, Homo sapiens
  misc_feature (21)..(21): n is a, c, g, or t
aacctggggtgccttgggtcngg

SEQ ID NO: 376 - DNA, 23 bp, Homo sapiens
  misc_feature (21)..(21): n is a, c, g, or t
ttggaaacctggggtgccttngg

SEQ ID NO: 377 - DNA, 23 bp, Homo sapiens
  misc_feature (21)..(21): n is a, c, g, or t
gtatcaatatatgatacaaangg

SEQ ID NO: 378 - DNA, 23 bp, Homo sapiens
  misc_feature (21)..(21): n is a, c, g, or t
caaggcaccccaggtttccangg

SEQ ID NO: 379 - DNA, 23 bp, Homo sapiens
  misc_feature (21)..(21): n is a, c, g, or t
cttggaaacctggggtgcctngg

SEQ ID NO: 380 - DNA, 23 bp, Homo sapiens
  misc_feature (21)..(21): n is a, c, g, or t
tcatcactcatttgaaaactngg

SEQ ID NO: 381 - DNA, 23 bp, Homo sapiens
  misc_feature (21)..(21): n is a, c, g, or t
caagttgatgtcctgacccangg

SEQ ID NO: 382 - DNA, 23 bp, Homo sapiens
  misc_feature (21)..(21): n is a, c, g, or t
gtcctgacccaaggcaccccngg

SEQ ID NO: 383 - DNA, 23 bp, Homo sapiens
  misc_feature (21)..(21): n is a, c, g, or t
atatttgatttgaagattcangg

SEQ ID NO: 384 - DNA, 23 bp, Homo sapiens
  misc_feature (21)..(21): n is a, c, g, or t
atatttgatttgaagattcangg

SEQ ID NO: 385 - DNA, 23 bp, Homo sapiens
  misc_feature (21)..(21): n is a, c, g, or t
acatataagctatttaaaaangg

SEQ ID NO: 386 - DNA, 23 bp, Homo sapiens
  misc_feature (21)..(21): n is a, c, g, or t
aaaagagaaagagactttcangg

SEQ ID NO: 387 - DNA, 23 bp, Homo sapiens
  misc_feature (21)..(21): n is a, c, g, or t
cttgatacaggtttaattcgngg

SEQ ID NO: 388 - DNA, 23 bp, Homo sapiens
  misc_feature (21)..(21): n is a, c, g, or t
acagctgacaggctcgacacngg

SEQ ID NO: 389 - DNA, 23 bp, Homo sapiens
  misc_feature (21)..(21): n is a, c, g, or t
gatgtttcccatcttgatacngg

SEQ ID NO: 390 - DNA, 23 bp, Homo sapiens
  misc_feature (21)..(21): n is a, c, g, or t
gtcgagcctgtcagctgtccngg

SEQ ID NO: 391 - DNA, 23 bp, Homo sapiens
  misc_feature (21)..(21): n is a, c, g, or t
caaaattcaagtgcacagcangg

SEQ ID NO: 392 - DNA, 23 bp, Homo sapiens
  misc_feature (21)..(21): n is a, c, g, or t
gaattttgcactcaggctggngg

SEQ ID NO: 393 - DNA, 23 bp, Homo sapiens
  misc_feature (21)..(21): n is a, c, g, or t
gaattaaacctgtatcaagangg

SEQ ID NO: 394 - DNA, 23 bp, Homo sapiens
  misc_feature (21)..(21): n is a, c, g, or t
aattaaacctgtatcaagatngg

SEQ ID NO: 395 - DNA, 23 bp, Homo sapiens
  misc_feature (21)..(21): n is a, c, g, or t
agttccattcattacctcacngg

SEQ ID NO: 396 - DNA, 23 bp, Homo sapiens
  misc_feature (21)..(21): n is a, c, g, or t
agagggtcatcacacacaagngg

SEQ ID NO: 397 - DNA, 23 bp, Homo sapiens
  misc_feature (21)..(21): n is a, c, g, or t
atacaagtccaggagatcttngg

SEQ ID NO: 398 - DNA, 23 bp, Homo sapiens
  misc_feature (21)..(21): n is a, c, g, or t
tcttgatacaggtttaattcngg

SEQ ID NO: 399 - DNA, 23 bp, Homo sapiens
  misc_feature (21)..(21): n is a, c, g, or t
ctgacctgtgaggtaatgaangg

SEQ ID NO: 400 - DNA, 23 bp, Homo sapiens
  misc_feature (21)..(21): n is a, c, g, or t
acatctaaaactttctcagangg

SEQ ID NO: 401 - DNA, 23 bp, Homo sapiens
  misc_feature (21)..(21): n is a, c, g, or t
gcaaaattcaagtgcacagcngg

SEQ ID NO: 402 - DNA, 23 bp, Homo sapiens
  misc_feature (21)..(21): n is a, c, g, or t
ggttgtgttgatacaagtccngg

SEQ ID NO: 403 - DNA, 23 bp, Homo sapiens
  misc_feature (21)..(21): n is a, c, g, or t
atcttgatacaggtttaattngg

SEQ ID NO: 404 - DNA, 23 bp, Homo sapiens
  misc_feature (21)..(21): n is a, c, g, or t
attcattacctcacaggtcangg

SEQ ID NO: 405 - DNA, 23 bp, Homo sapiens
  misc_feature (21)..(21): n is a, c, g, or t
aacatctaaaactttctcagngg

SEQ ID NO: 406 - DNA, 23 bp, Homo sapiens
  misc_feature (21)..(21): n is a, c, g, or t
agcagggaacaaagtcagcangg

SEQ ID NO: 407 - DNA, 23 bp, Homo sapiens
  misc_feature (21)..(21): n is a, c, g, or t
caacacaaccctgacctgtgngg

SEQ ID NO: 408 - DNA, 23 bp, Homo sapiens
  misc_feature (21)..(21): n is a, c, g, or t
cttgaattttgcactcaggcngg

SEQ ID NO: 409 - DNA, 23 bp, Homo sapiens
  misc_feature (21)..(21): n is a, c, g, or t
cattcattacctcacaggtcngg

SEQ ID NO: 410 - DNA, 23 bp, Homo sapiens
  misc_feature (21)..(21): n is a, c, g, or t
tgcacttgaattttgcactcngg

SEQ ID NO: 411 - DNA, 23 bp, Homo sapiens
  misc_feature (21)..(21): n is a, c, g, or t
tctcaaaaccaaagatctccngg

SEQ ID NO: 412 - protein, 30 aa, Artificial Sequence
  Description of Artificial Sequence Synthetic polypeptide
  MISC_FEATURE: This sequence may encompass 2-6 “Gly Gly Gly Gly Ser” repeating units
GGGGSGGGGSGGGGSGGGGSGGGGSGGGGS

SEQ ID NO: 413 - protein, 50 aa, Artificial Sequence
  Description of Artificial Sequence Synthetic polypeptide
  MISC_FEATURE: This sequence may encompass 1-3 “Gly Gly Gly Gly Ser” repeating units, 1 “Gly Gly Gly Gly Pro” repeating unit, and 1 “Gly Gly Gly Gly Cys” repeating unit.
GGGGSGGGGCGGGGSGGGGSGGGGPGGGGSGGGGSGGGGSGGGGCGGGGS

SEQ ID NO: 414 - protein, 20 aa, Artificial Sequence
  Description of Artificial Sequence Synthetic polypeptide
  MISC_FEATURE: This sequence may encompass 3-4 “Gly Gly Gly Gly Ser” repeating units
GGGGSGGGGSGGGGSGGGGS

SEQ ID NO: 415 - protein, 20 aa, Artificial Sequence
  Description of Artificial Sequence Synthetic polypeptide
GGGGSGGGGSGGGGSGGGGS

SEQ ID NO: 416 - protein, 50 aa, Artificial Sequence
  Description of Artificial Sequence Synthetic polypeptide
GGGGSGGGGSGGGGSGGGGSGGGGSGGGGSGGGGSGGGGSGGGGSGGGGS

SEQ ID NO: 417 - protein, 45 aa, Artificial Sequence
  Description of Artificial Sequence Synthetic polypeptide
GGGGSGGGGSGGGGSGGGGSGGGGPGGGGSGGGGSGGGGSGGGGS

(19) United States
(12) Patent Application Publication
(10) Pub. No.: US 2020/0109364 A1
(43) Pub. Date: Apr. 9, 2020

(54) METHODS FOR GENOME-EDITING AND ACTIVATION OF CELLS

(71) Applicant: Washington University, St. Louis, MO (US)

(72) Inventors: John DiPersio, St. Louis, MO (US); Matthew Cooper, St. Louis, MO (US)

(21) Appl. No.: 16/428,348

(22) Filed: May 31, 2019

Related U.S. Application Data
(60) Provisional application No. 62/678,886, filed on May 31, 2018.

(51) Int. Cl. C12N 5/0783; C12N 15/11; C12N 9/22; C12N 15/87; C12N 15/86; C12N 7/00; A61K 35/17

(52) U.S. Cl. CPC C12N 5/0636; C12N 15/11; C12N 9/22; C12N 15/87; C12N 15/86; C12N 2740/15043; A61K 35/17; C12N 2510/00; C12N 2310/20; C12N 2800/80; C12N 2501/998; C12N 7/00

(57) ABSTRACT

Disclosed herein are methods of genome-editing and transduction of T cells and methods of immunotherapy in using them. In particular, the disclosure relates to engineered chimeric antigen receptor (CAR)-bearing T cells and methods of using the same for the treatment of cancer.

This application claims the benefit of U.S. Provisional Application No. 62/678,886, filed May 31, 2018, the disclosure of which is hereby incorporated by reference in its entirety.

The instant application contains a Sequence Listing which has been submitted electronically in ASCII format and is hereby incorporated by reference in its entirety. Said ASCII copy, created on Aug. 16, 2019, is named WGN0004-201-US.txt and is 162,096 bytes in size.

Disclosed herein are methods of genome-editing and transduction of T cells and methods of immunotherapy in using them. In particular, the disclosure relates to engineered chimeric antigen receptor (CAR)-bearing T cells (CAR-T) methods of using the same for the treatment of T and B cell malignancies.

Chimeric antigen receptor T cell (CAR-T) immunotherapy is increasingly well known. T cells are genetically modified to express chimeric antigen receptors (CARs), which are fusion proteins comprised of an antigen recognition moiety and T cell activation domains. The CARs are designed to recognize antigens that are overexpressed on cancer cells. CAR-Ts demonstrate exceptional clinical efficacy against B cell malignancies, and two therapies, Kymriah™ (tisagenlecleucel, Novartis) and Yescarta™ (axicabtagene ciloleucel, Kite/Gilead), were recently approved by the FDA. Each of these therapies involves transduction of a CAR into each patient's own T cells, and adoptive cell transfer of disease-targeting autologous CAR-T cells into the patient. This process takes a significant amount of time and is extremely expensive.

Broad applicability of CAR-T therapy has been limited in two additional ways. First, the development of CAR-T cell therapy against T cell malignancies has proven problematic, in part due to the shared expression of target antigens between malignant T cells and effector T cells, because expression of target antigens on CAR-T cells may induce fratricide of CAR-T cells and loss of efficacy. Second, the use of T-cells other than an individual patient's own (allogenic) in CAR-T therapy may lead to allogenic reactivity including graft-versus-host disease.

Furthermore, the production of CAR-T cells is inefficient, and the end goal of inexpensive, readily-available adoptive cell transfer therapy including CAR-T therapy would be well-served by improved methods which increase expansion of allogeneic cells with the desired characteristics. Disclosed herein are such methods, and cells made by them.

### BRIEF DESCRIPTION OF THE DRAWINGS

FIG. 1 shows two embodiments of the alternative method of producing genome-edited CAR-T cells disclosed herein, wherein gene editing precedes activation. The top panel shows a flow diagram with alternative lengths of time for and between steps; the bottom panel give a more specific embodiment.

FIG. 2 shows T cells by flow cytometry to check for the deletion of T Cell Receptor (TCR) following genome editing of TRAC.

FIGS. 3-8 show the effect of increasing the time between genome editing (e.g., electroporation (EP) of Cas9 mRNA and gRNA) and activation (e.g., by stimulation with anti-CD3 and anti-CD28 mAbs) of T cells on proliferation of CD3⁺ and CD3⁻ T cells as measured by TCRα and CD3ε surface expression in T cells on day +4. In each of FIGS. 3-8, the top panel is a scatter plot of flow cytometry results showing TCR expression, specifically, TRAC expression (FL1-A by FITC (fluorescein isothiocyanate), vertical axis) against CD3 expression (FL6-A by APC (allophycocyanin), horizontal axis). The bottom panel shows the count of CD3⁺ and CD3⁻ cells (vertical axis) against CD3 antigen expression (FL6-A by APC, horizontal axis).

FIG. 3 shows proliferation of CD3⁺ and CD3⁻ cells when no genome editing (EP) is performed prior to activation, as measured by TCRα and CD3ε surface expression in T cells on day +4.

FIG. 4 shows proliferation of CD3⁺ and CD3⁻ cells when cells are activated immediately after genome editing (EP), i.e., with no deliberate delay, as measured by TCRα and CD3ε surface expression in T cells on day +4.

FIG. 5 shows proliferation of CD3⁺ and CD3⁻ cells when cells are activated 4 hours after genome editing (EP), as measured by TCRα and CD3ε surface expression in T cells on day +4.

FIG. 6 shows proliferation of CD3⁺ and CD3⁻ cells when cells are activated 8 hours after genome editing (EP), as measured by TCRα and CD3ε surface expression in T cells on day +4.

FIG. 7 shows proliferation of CD3⁺ and CD3⁻ cells when cells are activated 20 hours after genome editing (EP), as measured by TCRα and CD3ε surface expression in T cells on day +4.

FIG. 8 shows the kinetics of TRAC deletion in gene edited T cells.

FIG. 9 shows a theoretical T cell activation window.

FIG. 10 shows the kinetics of T cell expansion in gene edited T cells. The top panel shows absolute cell counts; the bottom panel shows fold expansion.

Additional description of the figures is given below.

### DETAILED DESCRIPTION

Accordingly, disclosed herein as Embodiment 1 is a method of making a population of genome-edited immune effector cells, comprising the steps of:
a. editing the genome of a population of T-cell receptor (TCR) bearing immune effector cells;
b. activating the immune effector cell population; and
c. expanding the population of genome-edited immune effector cells.

Conventional methods teach that it is necessary to activate cells before editing to expand their population. As shown herein, the opposite sequence is in some circumstances more effective, enabling efficient genome editing of cells and expansion of the edited population.

The editing may take many forms. Either protein or a nucleic acid, particularly RNA, may be transduced into a cell, for a range of purposes. Gene deletion or suppression, insertion or expression of a chimeric antigen receptor (CAR), and expression of a protein or short hairpin RNA (shRNA) may all be effected. Techniques such as CRISPR (particularly using Cas9 and guide RNA), editing with zinc finger nucleases (ZFNs) and transcription activator-like effector nucleases (TALENs) may be used; vectors may also deliver constructs for expression and/or genetic integration. Preceding or subsequent editing steps may also be performed attendant to the core editing followed by activation.

The following disclosure will detail embodiments, alternatives, and applications of the method, as well as engineered cells made by the method and the use sch cells in, for example, immunotherapy and adoptive cell transfer for the treatment of diseases. Accordingly, provided herein are the following embodiments.

### Embodiment 2

The method as recited in Embodiment 1, wherein the T-cell receptor (TCR) bearing immune effector cells are transduced with at least one chimeric antigen receptor (CAR) that recognize(s) one or more proteins.

### Embodiment 3

The method as recited in Embodiment 2, wherein the genome editing step (a) comprises transducing the immune effector cell population with the one or more CARs.

### Embodiment 4

The method as recited in Embodiment 2, comprising an additional step to be performed between steps (b) and (c), of transducing the immune effector cell population with the one or more CARs.

### Embodiment 5

A method of making a population of genome-edited, chimeric antigen receptor (CAR) bearing immune effector cells, comprising the steps of:
a. editing the genome of a population of T-cell receptor (TCR) bearing immune effector cells;
b. activating the immune effector cell population;
c. transducing the immune effector cell population with at least one chimeric antigen receptor (CAR) that recognize(s) one or more proteins; and
d. expanding the population of genome-edited, chimeric antigen receptor bearing immune effector cells.

### Embodiment 6

The method as recited in any of Embodiments 1-5, wherein the TCR bearing immune effector cells are purified.

### Embodiment 7

The method as recited in any of Embodiments 1-6, wherein the immune effector cells are T cells.

### Embodiment 8

The method as recited in any of Embodiments 1-7, wherein the one or more proteins recognized by the chimeric antigen receptor (CAR) is/are chosen from antigens and cell surface proteins.

### Embodiment 9

The method as recited in any of Embodiments 1-8, wherein genome is edited using a CRISPR associated protein (Cas-CRISPR), a transcription activator-like effector nuclease (TALEN), or a zinc-finger nuclease (ZFN) delivered into the cell.

### Embodiment 10

The method as recited in Embodiment 9, wherein genome is edited using Cas-CRISPR.

### Embodiment 11

The method as recited in Embodiment 10, wherein the genome is edited using Cas9-CRISPR.

### Embodiment 12

The method as recited in Embodiment 11, wherein the Cas9 is delivered into the cell as mRNA or protein.

### Embodiment 13

The method as recited in Embodiment 12, wherein the Cas9 is delivered into the cell as mRNA.

### Embodiment 14

The method as recited in Embodiment 12, wherein the Cas9 is delivered into the cell as protein.

### Embodiment 15

The method as recited in any of Embodiments 9-14, wherein a guide RNA (gRNA) targeting the gene to be edited is delivered contemporaneously with the Cas9.

### Embodiment 16

The method as recited in any of Embodiments 1-16, wherein genome is edited by transducing the cells with a nucleic acid encoding a protein or shRNA.

### Embodiment 17

The method as recited in Embodiment 16, wherein the transducing is by a virus or viral vector.

### Embodiment 18

The method as recited in Embodiment 17, wherein the transducing is by a lentiviral vector.

### Embodiment 19

The method as recited in Embodiment 17, wherein the transducing is by an adeno-associated virus.

### Embodiment 20

The method as recited in any of Embodiments 9-19, wherein the delivery or transducing is by electroporation.

### Embodiment 21

The method as recited in Embodiment 1-20, wherein the genome editing comprises deleting or suppressing the expression of one or more antigens or cell surface proteins.

### Embodiment 22

The method as recited in Embodiment 21, wherein a cell surface protein deleted/suppressed is the major histocompatibility complex I (MHCI), or a subunit thereof.

### Embodiment 23

The method as recited in Embodiment 22, wherein a cell surface protein deleted/suppressed is (32 microglobulin.

### Embodiment 24

The method as recited in Embodiment 21, wherein a cell surface protein deleted/suppressed is the T Cell Receptor (TCR), or a subunit thereof.

### Embodiment 25

The method as recited in Embodiment 24, wherein a cell surface protein deleted/suppressed is chosen from TRAC (TCR-α), TCR-β, CD3ε, CD3ζ; CD3δ, and CD3γ.

### Embodiment 26

The method as recited in Embodiment 25, wherein a cell surface protein deleted/suppressed is TRAC.

### Embodiment 27

The method as recited in Embodiment 21, wherein a cell surface protein deleted/suppressed is a protein which prevents T cell exhaustion.

### Embodiment 28

The method as recited in Embodiment 27, wherein a cell surface protein which prevents T cell exhaustion is an immunological checkpoint on a T cell.

### Embodiment 29

The method as recited in Embodiment 28, wherein the surface protein which prevents T cell exhaustion is chosen from PD-1, LAG-3, Tim-3, and CTLA-4.

### Embodiment 30

The method as recited in Embodiment 21, wherein the genome editing comprises deleting or suppressing the expression of one or more secretable proteins.

### Embodiment 31

The method as recited in Embodiment 30, wherein the secretable protein is a cytokine.

### Embodiment 32

The method as recited in Embodiment 31, wherein the cytokine is chosen from MCP1 (CCL2), MCP-2, GM-CSF, G-CSF, M-CSF, 11-4, and IFNγ.

### Embodiment 33

The method as recited in Embodiment 32, wherein the cytokine is GM-CSF.

### Embodiment 34

The method as recited in Embodiment 31, wherein the secretable protein is a transcription factor.

### Embodiment 35

The method as recited in Embodiment 32, wherein the transcription factor is chosen from AHR, BCL6, FOXP3, GATA3, MAF, RORC, SPI1, TBX21.

### Embodiment 36

The method as recited in Embodiment 21, wherein the cell surface protein or antigen deleted/suppressed is the target of the CAR.

### Embodiment 37

The method as recited in Embodiment 21, wherein the genome editing comprises transduction to express a protein expression blocker (PEBL).

### Embodiment 38

The method as recited in Embodiment 21, wherein the genome editing comprises transduction to express a shRNA.

### Embodiment 39

The method as recited in any of Embodiments 1-38, wherein the cells are allowed to rest after editing for up to 48 hours before activation.

### Embodiment 40

The method as recited in any of Embodiments 1-38, wherein the cells are allowed to rest after editing for up to 24 hours before activation.

### Embodiment 41

The method as recited in any of Embodiments 1-38, wherein the cells are allowed to rest after editing for up to 8 hours before activation.

### Embodiment 42

The method as recited in any of Embodiments 1-38, wherein the cells are allowed to rest after editing for up to 4 hours before activation.

### Embodiment 43

The method as recited in any of Embodiments 1-38, wherein the cells are allowed to rest after editing for between 24 and 48 hours before activation

### Embodiment 44

The method as recited in any of Embodiments 1-38, wherein the cells are activated immediately after genome editing.

### Embodiment 45

The method as recited in any of Embodiments 1-44, wherein the activating of the immune effector cells is done by exposing the cell population to anti-CD3 antibodies and anti-CD28 antibodies, or a functional fragment of either of the foregoing.

### Embodiment 46

The method as recited in any of Embodiments 1-44, wherein the activating of the immune effector cells is done by exposing the cell population to anti-CD3, anti-CD28, and anti-CD2 antibodies, or a functional fragment of either of the foregoing.

### Embodiment 47

The method as recited in any of Embodiments 45-46, wherein the antibodies are affixed to beads.

### Embodiment 48

The method as recited in any of Embodiments 1-47, wherein the genome-edited cells are activated for up to five days.

### Embodiment 49

The method as recited in any of Embodiments 1-47, wherein the genome-edited cells are activated for up to two days.

### Embodiment 50

The method as recited in any of Embodiments 1-47, wherein the genome-edited cells are activated for up to one day.

### Embodiment 51

The method as recited in any of Embodiments 45-50, wherein the anti-CD3 antibodies, anti-CD28 antibodies, and/or anti-CD2 antibodies are removed from the cell population by application of a magnetic field or by washing.

### Embodiment 52

The method as recited in any of Embodiments 2-51, wherein the CAR is transduced into the cell less than 48 hours post-activation.

### Embodiment 53

The method as recited in any of Embodiments 2-51, wherein the CAR is transduced into the cell less than 24 hours post-activation.

### Embodiment 54

The method as recited in any of Embodiments 2-53, wherein the CAR is transduced into the cell using a lentiviral vector encoding the CAR.

### Embodiment 55

The method as recited in any of Embodiments 1-54, wherein the population of cells is expanded for less than 20 days.

### Embodiment 56

The method as recited in Embodiments 1-54, wherein the population of cells is expanded for less than 12 days.

### Embodiment 57

The method as recited in Embodiments 1-54, wherein the population of cells is expanded for less than 10 days.

### Embodiment 58

The method as recited in Embodiments 1-54, wherein the population of cells is expanded for less than 8 days.

### Embodiment 59

The method as recited in Embodiments 1-54, wherein the population of cells is expanded for less than 6 days.

### Embodiment 60

The method as recited in any of Embodiments 1-59, performed at a temperature of between about 25° C. and about 40° C.

### Embodiment 61

The method as recited in any of Embodiments 1-59, performed at a temperature of between about 30° C. and about 37° C.

### Embodiment 62

The method as recited in any of Embodiments 1-59, performed at about 37° C.

### Embodiment 63

The method as recited in any of Embodiments 1-59, performed at about 30° C.

### Embodiment 64

The method as recited in any of Embodiments 1-63, comprising the additional step of analyzing the cells by flow cytometry to confirm expression of the CAR (or CARs if multiple were transduced in) and/or expression of a transduced protein and/or expression (or lack thereof, i.e., deletion or suppression) of a protein.

### Embodiment 65

The method as recited in any of Embodiments 24-64, comprising the additional step of depleting TCR⁺ cells.

### Embodiment 66

The method as recited in any of Embodiments 1-65, wherein the immune effector cells to be used are harvested from a healthy donor (or from cord blood, or from PBMCs).

### Embodiment 67

The method as recited in Embodiment 66, wherein the donor is a human.

### Embodiment 68

The method as recited in any of Embodiments 2-67, wherein the chimeric antigen receptor(s) specifically binds at least one antigen expressed on a malignant cell.

### Embodiment 69

The method as recited in Embodiment 68, wherein the one or more antigens expressed on a malignant cell is chosen from BCMA, CS1, CD38, CD138, CD19, CD33, CD123, CD371, CD117, CD135, Tim-3, CD5, CD7, CD2, CD4, CD3, CD79A, CD79B, APRIL, CD56, and CD1a.

### Embodiment 70

The method as recited in Embodiment 68, wherein the chimeric antigen receptor(s) specifically binds at least one antigen expressed on a malignant T cell.

### Embodiment 71

The method as recited in Embodiment 70, wherein the antigen expressed on a malignant T cell is chosen from CD2, CD3, CD4, CD5, CD7, TCRA, and TCRβ.

### Embodiment 72

The method as recited in Embodiment 68, wherein the chimeric antigen receptor specifically binds at least one antigen expressed on a malignant plasma cell.

### Embodiment 73

The method as recited in Embodiment 72, wherein the antigen expressed on a malignant plasma cell is chosen from BCMA, CS1, CD38, CD79A, CD79B, CD138, and CD19.

### Embodiment 74

The method as recited in Embodiment 68, wherein the chimeric antigen receptor(s) specifically binds at least one antigen expressed on a malignant B cell.

### Embodiment 75

The method as recited in Embodiment 74, wherein the antigen expressed on a malignant B cell is chosen from CD19, CD20, CD21, CD22, CD23, CD24, CD25, CD27, CD38, and CD45.

### Embodiment 76

The method as recited in Embodiment 75, wherein the antigen expressed on a malignant B cell is chosen from CD19, CD20, CD22, CD24, CD38, and CD45.

### Embodiment 77

The method as recited in Embodiment 68, wherein the chimeric antigen receptor specifically binds at least one antigen expressed on a malignant mesothelial cell.

### Embodiment 78

The method as recited in Embodiment 77, wherein the antigen expressed on a malignant mesothelial cell is mesothelin.

### Embodiment 79

A method of making a population of genome-edited CAR-T cells comprising the steps of:
a. deleting or suppressing the expression of one or more antigen(s) or cell surface protein(s) in a T cell population, using Cas9-CRISPR and gRNA targeting the gene(s) encoding the antigen(s) or cell surface protein(s);
b. activating the T cell population;
c. transducing the T cell population with a chimeric antigen receptor that recognizes one or more antigens or cell surface proteins; and
d. expanding the population of CAR-T cells.

### Embodiment 80

The method as recited in Embodiment 79, wherein a cell surface protein or antigen deleted/suppressed is chosen from TRAC (TCR-α), TCR-β, CD3ε, CD3ζ, CD3δ, and CD3γ.

### Embodiment 81

The method as recited in Embodiment 80, wherein a cell surface protein or antigen deleted/suppressed is TRAC.

### Embodiment 82

A method of making a population of genome-edited CAR-T cells that are deficient in T Cell Receptor (TCR) signaling comprising the steps of:
a. deleting or suppressing the expression of the T cell receptor (TCR) or a subunit thereof and, optionally, deleting or suppressing the expression of one or more antigen(s) or cell surface protein(s) in a T cell population, using Cas9-CRISPR and gRNA targeting the gene(s) encoding the antigen(s) or cell surface protein(s);
b. activating the T cell population;
c. transducing the T cell population with a chimeric antigen receptor that recognizes one or more antigens or cell surface proteins; and
d. expanding the population of TCR-deficient CAR-T cells.

### Embodiment 83

The method as recited in Embodiment 82, wherein the TCR subunit deleted/suppressed is chosen from TRAC (TCR-α), TCR-β, CD3ε, CD3ζ, CD3δ, and CD3γ.

### Embodiment 84

The method as recited in Embodiment 82, wherein the TCR subunit deleted/suppressed is TRAC.

### Embodiment 85

The method as recited in any of Embodiments 79-83, wherein the Cas9 is delivered into the cell as mRNA or protein.

### Embodiment 86

The method as recited in Embodiment 85, wherein the Cas9 is delivered into the cell as mRNA.

### Embodiment 87

The method as recited in Embodiment 86, wherein the Cas9 is delivered into the cell as protein.

### Embodiment 88

The method as recited in any of Embodiments 82-87, comprising deleting or suppressing the expression of one or more antigen(s) or cell surface protein(s).

### Embodiment 89

The method as recited in Embodiment 88, wherein the cell surface protein or antigen deleted/suppressed is the target of the CAR.

### Embodiment 90

The method as recited in any of Embodiments 79-89, wherein genome is edited by transducing the cells with a nucleic acid encoding a protein or shRNA.

### Embodiment 91

The method as recited in Embodiment 90, wherein the transducing is by a virus or viral vector.

### Embodiment 92

The method as recited in Embodiment 91, wherein the transducing is by a lentiviral vector.

### Embodiment 93

The method as recited in Embodiment 91, wherein the transducing is by an adeno-associated virus.

### Embodiment 94

The method as recited in any of Embodiments 79-93, wherein the delivery or transducing is by electroporation.

### Embodiment 95

The method as recited in any of Embodiments 79-94, wherein a cell surface protein deleted/suppressed is the major histocompatibility complex I (MHCI), or a subunit thereof.

### Embodiment 96

The method as recited in Embodiment 94, wherein the subunit is (32 microglobulin.

### Embodiment 97

The method as recited in any of Embodiments 79-93, wherein a cell surface protein deleted/suppressed is a protein which prevents T cell exhaustion.

### Embodiment 98

The method as recited in Embodiment 97, wherein a cell surface protein which prevents T cell exhaustion is an immunological checkpoint on a T cell.

### Embodiment 99

The method as recited in Embodiment 98, wherein the surface protein which prevents T cell exhaustion is chosen from PD-1, LAG-3, Tim-3, and CTLA-4.

### Embodiment 100

The method as recited in any of Embodiments 79-93, wherein the genome editing comprises transduction to express a protein expression blocker (PEBL).

### Embodiment 101

The method as recited in any of Embodiments 79-100, wherein the chimeric antigen receptor(s) specifically binds at least one antigen expressed on a malignant cell.

### Embodiment 102

The method as recited in Embodiment 101, wherein the one or more antigens expressed on a malignant cell is chosen from BCMA, CS1, CD38, CD138, CD19, CD33, CD123, CD371, CD117, CD135, Tim-3, CD5, CD7, CD2, CD4, CD3, CD79A, CD79B, APRIL, CD56, and CD1a.

### Embodiment 103

The method as recited in Embodiment 101, wherein the chimeric antigen receptor(s) specifically binds at least one antigen expressed on a malignant T cell.

### Embodiment 104

The method as recited in Embodiment 103, wherein the antigen expressed on a malignant T cell is chosen from CD2, CD3, CD4, CD5, CD7, TCRA, and TCRβ.

### Embodiment 105

The method as recited in Embodiment 101, wherein the chimeric antigen receptor specifically binds at least one antigen expressed on a malignant plasma cell.

### Embodiment 106

The method as recited in Embodiment 105, wherein the antigen expressed on a malignant plasma cell is chosen from BCMA, CS1, CD38, CD79A, CD79B, CD138, and CD19.

### Embodiment 107

The method as recited in Embodiment 101, wherein the chimeric antigen receptor(s) specifically binds at least one antigen expressed on a malignant B cell.

### Embodiment 108

The method as recited in Embodiment 107, wherein the antigen expressed on a malignant B cell is chosen from CD19, CD20, CD21, CD22, CD23, CD24, CD25, CD27, CD38, and CD45.

### Embodiment 109

The method as recited in Embodiment 108, wherein the antigen expressed on a malignant B cell is chosen from CD19, CD20, CD22, CD24, CD38, and CD45; or is chosen from CD19 and CD20.

### Embodiment 110

The method as recited in Embodiment 101, wherein the chimeric antigen receptor specifically binds at least one antigen expressed on a malignant mesothelial cell.

### Embodiment 111

The method as recited in Embodiment 110, wherein the antigen expressed on a malignant mesothelial cell is mesothelin.

### Embodiment 112

A method of making a population of chimeric antigen receptor T (CAR-T) cells in which the CAR targets CD7, in which TRAC and CD7 are deleted (UCART7 cells), comprising the steps of:
a. editing the CD7 and TRAC genes in of a population of T-cells from a healthy human donor to delete/suppress CD7 and TRAC, using Cas9-CRISPR and gRNA targeting the gene encoding the antigen(s) or cell surface protein(s);
b. activating the T cell population;
c. transducing the T cell population with a chimeric antigen receptor that recognizes CD7; and
d. expanding the population of UCART7 cells.

### Embodiment 113

A method of making a population of chimeric antigen receptor T (CAR-T) cells in which the CAR is a tandem CAR that targets CD2 and CD3ε, in which CD3ε and CD2 are deleted (tUCART2/3 cells), comprising the steps of:
a. editing the CD2 and CD3ε genes in of a population of T-cells from a healthy human donor to delete/suppress CD2 and CD3ε, using Cas9-CRISPR and gRNA targeting the gene encoding the antigen(s) or cell surface protein(s);
b. activating the T cell population;
c. transducing the T cell population with a tandem chimeric antigen receptor that recognizes CD and CD3ε; and
d. expanding the population of tUCART2/3 cells.

### Embodiment 114

The method as recited in any of Embodiments 79-113, wherein the Cas9 is delivered into the cell as mRNA or protein.

### Embodiment 115

The method as recited in Embodiment 114, wherein the Cas9 is delivered into the cell as mRNA.

### Embodiment 116

The method as recited in Embodiment 114, wherein the Cas9 is delivered into the cell as protein.

### Embodiment 117

The method as recited in any of Embodiments 79-116, comprising deleting or suppressing the expression of one or more antigen(s), cell surface protein(s), or secretable proteins.

### Embodiment 118

The method as recited in any of Embodiments 79-119, wherein genome is edited by transducing the cells with a nucleic acid encoding a protein or shRNA.

### Embodiment 119

The method as recited in Embodiment 118, wherein the transducing is by a virus or viral vector.

### Embodiment 120

The method as recited in Embodiment 119, wherein the transducing is by a lentiviral vector.

### Embodiment 121

The method as recited in Embodiment 118, wherein the transducing is by an adeno-associated virus.

### Embodiment 122

The method as recited in any of Embodiments 79-121, wherein the delivery or transducing is by electroporation.

### Embodiment 123

The method as recited in any of Embodiments 117-122, wherein a cell surface protein deleted/suppressed is the major histocompatibility complex I (MHCI), or a subunit thereof.

### Embodiment 124

The method as recited in Embodiment 123, wherein the subunit is (32 microglobulin.

### Embodiment 125

The method as recited in any of Embodiments 117-122, wherein a cell surface protein deleted/suppressed is a protein which prevents T cell exhaustion.

### Embodiment 126

The method as recited in Embodiment 125, wherein a cell surface protein which prevents T cell exhaustion is an immunological checkpoint on a T cell.

### Embodiment 127

The method as recited in Embodiment 126, wherein the surface protein which prevents T cell exhaustion is chosen from PD-1, LAG-3, Tim-3, and CTLA-4.

### Embodiment 128

The method as recited in any of Embodiments 117-122, wherein the genome editing comprises transduction to express a protein expression blocker (PEBL).

### Embodiment 129

The method as recited in any of any of Embodiments 79-128, wherein the cells are allowed to rest after editing for up to 48 hours before activation.

### Embodiment 130

The method as recited in any of Embodiments 79-128, wherein the cells are allowed to rest after editing for up to 24 hours before activation.

### Embodiment 131

The method as recited in any of Embodiments 79-128, wherein the cells are allowed to rest after editing for up to 8 hours before activation.

### Embodiment 132

The method as recited in any of Embodiments 79-128, wherein the cells are allowed to rest after editing for up to 4 hours before activation.

### Embodiment 133

The method as recited in any of Embodiments 79-128, wherein the cells are allowed to rest after editing for between 24 and 48 hours before activation

### Embodiment 134

The method as recited in any of Embodiments 79-128, wherein the cells are activated immediately after genome editing.

### Embodiment 135

The method as recited in any of Embodiments 79-134, wherein the activating of the immune effector cells is done by exposing the cell population to anti-CD3 antibodies and anti-CD28 antibodies, or a functional fragment of either of the foregoing.

### Embodiment 136

The method as recited in any of Embodiments 79-134, wherein the activating of the immune effector cells is done by exposing the cell population to anti-CD3, anti-CD28, and anti-CD2 antibodies, or a functional fragment of either of the foregoing.

### Embodiment 137

The method as recited in any of Embodiments 107-136, wherein the antibodies are affixed to beads.

### Embodiment 138

The method as recited in any of Embodiments 79-137, wherein the genome-edited cells are activated for up to five days.

### Embodiment 139

The method as recited in any of Embodiments 79-137, wherein the genome-edited cells are activated for up to two days.

### Embodiment 140

The method as recited in any of Embodiments 79-137, wherein the genome-edited cells are activated for up to one day.

### Embodiment 141

The method as recited in any of Embodiments 79-137, wherein the anti-CD3 antibodies, anti-CD28 antibodies, and/or anti-CD2 antibodies are removed from the cell population by application of a magnetic field or by washing.

### Embodiment 142

The method as recited in any of Embodiments 79-141, wherein the CAR is transduced into the cell less than 48 hours post-activation.

### Embodiment 143

The method as recited in any of Embodiments 79-141, wherein the CAR is transduced into the cell less than 24 hours post-activation.

### Embodiment 144

The method as recited in any of Embodiments 79-143, wherein the CAR is transduced into the cell using a lentiviral vector encoding the CAR.

### Embodiment 145

The method as recited in any of Embodiments 79-144, wherein the population of cells is expanded for less than 20 days.

### Embodiment 146

The method as recited in Embodiments 79-144, wherein the population of cells is expanded for less than 12 days.

### Embodiment 147

The method as recited in Embodiments 79-144, wherein the population of cells is expanded for less than 10 days.

### Embodiment 148

The method as recited in Embodiments 79-144, wherein the population of cells is expanded for less than 8 days.

### Embodiment 149

The method as recited in Embodiments 79-144, wherein the population of cells is expanded for less than 6 days.

### Embodiment 150

The method as recited in any of Embodiments 79-149, performed at a temperature of between about 25° C. and about 40° C.

### Embodiment 151

The method as recited in any of Embodiments 79-149, performed at a temperature of between about 30° C. and about 37° C.

### Embodiment 152

The method as recited in any of Embodiments 79-149, performed at about 37° C.

### Embodiment 153

The method as recited in any of Embodiments 79-149, performed at about 30° C.

### Embodiment 154

The method as recited in any of Embodiments 79-153, comprising the additional step of analyzing the cells by flow cytometry to confirm expression of the CAR (or CARs if multiple were transduced in) and/or expression of a transduced protein and/or expression (or lack thereof, i.e., deletion or suppression) of a protein.

### Embodiment 155

The method as recited in any of Embodiments 79-154, comprising the additional step of depleting TCR⁺ cells.

### Embodiment 156

The method as recited in any of Embodiments 79-155, wherein the immune effector cells to be used are harvested from a healthy donor (or from cord blood, or from PBMCs).

### Embodiment 157

The method as recited in Embodiment 156, wherein the donor is a human.

### Embodiment 158

A population of genome-edited, chimeric antigen receptor bearing immune effector cells made by the method as recited in any of Embodiments 1-157.

### Embodiment 159

The genome-edited, chimeric antigen receptor bearing immune effector cells as recited in Embodiment 158, wherein the chimeric antigen receptor bearing immune effector cells further comprise a suicide gene.

### Embodiment 160

The genome-edited, chimeric antigen receptor bearing immune effector cells as recited in any of Embodiments 158-159, wherein endogenous T cell receptor mediated signaling is blocked in the cell.

### Embodiment 161

The genome-edited, chimeric antigen receptor bearing immune effector cell as recited in Embodiment 160, wherein the genome-edited, chimeric antigen receptor bearing immune effector cells do not induce alloreactivity or graft-versus-host disease.

### Embodiment 162

The genome-edited, chimeric antigen receptor bearing immune effector cells as recited in Embodiment 160 or 161, wherein the cell does not induce fratricide.

### Embodiment 163

The genome-edited, chimeric antigen receptor bearing immune effector cells as recited in any of Embodiments 158-162, which are a dual-CAR or tandem-CAR bearing, genome-edited immune effector cells.

### Embodiment 164

A therapeutic composition comprising the population of genome-edited, chimeric antigen receptor bearing immune effector cells as recited in any of Embodiments 158-162, and at least one therapeutically acceptable carrier and/or adjuvant.

### Embodiment 165

A method of treatment of cancer, autoimmune disease, or infectious disease in a subject on need thereof comprising administering to the subject a population of genome-edited immune effector cells, genome-edited CAR-T cells, or genome-edited tandem CAR-T cells as recited in any of Embodiments 1-157.

### Embodiment 166

The method as recited in Embodiment 165, wherein the method is for the treatment of cancer.

### Embodiment 167

The method as recited in Embodiment 166, wherein the cancer is a hematologic malignancy.

### Embodiment 168

The method as recited in Embodiment 167, wherein the hematologic malignancy is chosen from leukemia, lymphoma, multiple myeloma.

### Embodiment 169

The method as recited in Embodiment 167, wherein the hematologic malignancy is Hodgkin's lymphoma.

### Embodiment 170

The method as recited in Embodiment 167, wherein the hematologic malignancy is a B-cell lymphoma.

### Embodiment 171

The method as recited in Embodiment 170, wherein the B-cell lymphoma is chosen from diffuse large B-cell lymphoma (DLBCL), chronic lymphocytic leukemia (CLL)/small lymphocytic lymphoma (SLL), and B cell-precursor acute lymphoblastic leukemia (ALL).

### Embodiment 172

The method as recited in Embodiment 167, wherein the hematologic malignancy is a T-cell lymphomas.

### Embodiment 173

The method as recited in Embodiment 172, wherein the T-cell lymphoma is chosen from T-cell acute lymphoblastic leukemia/lymphoma (T-ALL), peripheral T-cell lymphoma (PTCL), T-cell chronic lymphocytic leukemia (T-CLL), and Sezary syndrome.

### Embodiment 174

The method as recited in Embodiment 167, wherein the hematologic malignancy is a leukemia.

### Embodiment 175

The method as recited in Embodiment 174, wherein the leukemia is chosen from Acute myeloid (or myelogenous) leukemia (AML), chronic myeloid (or myelogenous) leukemia (CML), acute lymphocytic (or lymphoblastic) leukemia (ALL), chronic lymphocytic leukemia (CLL) hairy cell leukemia (sometimes classified as a lymphoma).

### Embodiment 176

The method as recited in Embodiment 167, wherein the hematologic malignancy is a plasma cell malignancy.

### Embodiment 177

The method as recited in Embodiment 176, wherein the hematologic malignancy is a plasma cell malignancy is chosen from lymphoplasmacytic lymphoma, plasmacytoma, and multiple myeloma.

### Embodiment 178

The method as recited in Embodiment 165, wherein the cancer is a solid tumor.

Disclosed herein is a method of making a population of genome-edited CAR-T cells comprising the steps of deleting or suppressing the expression of one or more antigens or cell surface proteins in a T cell population, activating the T cell population and transducing the T cell population with a chimeric antigen receptor that recognizes one or more antigens or cell surface proteins; and expanding the population of CAR-T cells.

In certain embodiments, the transduction step utilizes a viral or non-viral vector.

In certain embodiments, the chimeric antigen receptor specifically binds at least one antigen expressed on a malignant T cell.

In certain embodiments, the antigen is chosen from CD2, CD3ε, CD4, CD5, CD7, TCRA, and TCRβ.

In certain embodiments, the cell surface protein is an immunological checkpoint on a T cell which is chosen from but not limited to PD-1, LAG-3, Tim-3, and CTLA-4.

In certain embodiments, the chimeric antigen receptor specifically binds at least one antigen expressed on a malignant plasma cell.

Also disclosed is a CAR-T cell further comprising a suicide gene.

Also disclosed is a CAR-T cell where the endogenous T cell receptor mediated signaling is blocked.

Also disclosed is a CAR-T cell that does not induce alloreactivity or graft-versus-host disease.

In certain embodiments, the CAR-T cells do not induce fratricide.

In certain embodiments, a dual or tandem CAR-T cell as recited in the methods is disclosed.

In certain embodiments, is a therapeutic composition comprising the population of CAR-T cells and at least one therapeutically acceptable carrier and/or adjuvant.

Also disclosed is a method of treatment of a solid tumor in a patient comprising administering a population of genome-edited CAR-T cells, dual CAR-T cells, tandem CAR-T cells or the therapeutic composition to a patient in need thereof.

Also disclosed is a method of treatment of a hematologic malignancy in a patient comprising administering a population of genome-edited CAR-T cells, dual CAR-T cells, tandem CAR-T cells or the therapeutic composition to a patient in need thereof.

In certain embodiments, the hematologic malignancy is a T-cell malignancy.

In certain embodiments, the T cell malignancy is T-cell acute lymphoblastic leukemia (T-ALL).

In certain embodiments, T cell malignancy is non-Hodgkin's lymphoma.

In certain embodiments, the hematologic malignancy is a B-cell malignancy.

In certain embodiments, the B-cell malignancy is a B-cell lymphoma.

In certain embodiments, the B-cell malignancy is a B-cell leukemia.

In certain embodiments, the hematologic malignancy is a myeloid malignancy.

In certain embodiments, the myeloid malignancy is acute myeloid leukemia.

Also disclosed is a method of making a population of genome-edited CAR-T cells that are deficient in T Cell Receptor (TCR) signaling comprising the steps of deleting or suppressing the expression of one or more antigens or cell surface proteins in a T cell population, activating the T cell population, transducing the T cell population with a chimeric antigen receptor that recognizes one or more antigens or cell surface proteins, and expanding the population of CAR-T cells.

### Genome-Edited T Cells and CAR-T Cells

The present disclosure provides chimeric antigen receptor-bearing immune effector cells such as T cells (CAR-T cells), pharmaceutical compositions comprising them, methods of immunotherapy for the treatment of cancer, for example hematologic malignancies.

A CAR-T cell is a T cell which expresses a chimeric antigen receptor. The T cell expressing a CAR molecule may be a helper T cell, a cytotoxic T cell, a viral-specific cytotoxic T cell, a memory T cell, or a gamma delta (γδ) T cell.

A chimeric antigen receptor (CAR), is a recombinant fusion protein comprising: 1) an extracellular ligand-binding domain, i.e., an antigen-recognition domain, 2) a transmembrane domain, and 3) a signaling transducing domain.

The extracellular ligand-binding domain is an oligo- or polypeptide that is capable of binding a ligand. Preferably, the extracellular ligand-binding domain will be capable of interacting with a cell surface molecule which may be an antigen, a receptor, a peptide ligand, a protein ligand of the target, or a polypeptide of the target. The extracellular ligand-binding domain can specifically bind to an antigen with an affinity constant or affinity of interaction (K_{D}) between about 0.1 pM to about 10 pM, to about 0.1 pM to about 1 pM, or more preferably to about 0.1 pM to about 100 nM. Methods for determining the affinity constant or affinity of interaction (K_{D}) are well-known in the art. In some instances, the extracellular ligand-binding domain is chosen to recognize a ligand that acts as a cell surface marker on target cells associated with particular disease states.

In one embodiment, the extracellular ligand-binding domain comprises a single chain antibody fragment (scFv) comprising the light (V_{L}) and the heavy (V_{H}) variable fragment joined by a linker (e.g., GGGGS₍₂₋₆₎) (SEQ ID NO:412) and confers specificity for either a T cell antigen or an antigen that is not specific to a T cell. In one embodiment, the chimeric antigen receptor of a CAR-T cell may bind to an T cell-specific antigen expressed or overexpressed on a malignant T cell for which a CAR-T cell is deficient in the antigen (e.g., a genome-edited CAR-T cell).

Non-limiting examples of CAR-targeted antigens expressed on malignant T cells include CD5, CD7, CD2, CD4, and CD3.

Non-limiting examples of CAR-targeted antigens expressed on the surface of leukemia cells (e.g., abnormal myeloblasts, red blood cells, or platelets) include CD123 (IL3RA), CD371 (CLL-1; CLEC12A), CD117 (c-kit), and CD135 (FLT3), CD7 and Tim3. A CAR may be constructed with an extracellular ligand-binding domain to target these antigens for treatment of leukemia, i.e., acute myeloid leukemia (AML).

Non-limiting examples of CAR-targeted antigens expressed on the surface of a multiple myeloma cell (e.g., a malignant plasma cell) include BCMA, CS1, CD38, CD79A, CD79B, CD138, and CD19. A CAR may be constructed with an extracellular ligand-binding domain to target these antigens for treatment of multiple myeloma. In another embodiment, the CAR may be constructed with a portion of the APRIL protein, targeting the ligand for the B-Cell Maturation Antigen (BCMA) and Transmembrane Activator and CAML Interactor (TACI), effectively co-targeting both BCMA and TACI for the treatment of multiple myeloma. A signal peptide directs the transport of a secreted or transmembrane protein to the cell membrane and/or cell surface to allow for correct localization of the polypeptide. Particularly, the signal peptide of the present disclosure directs the appended polypeptide, i.e., the CAR receptor, to the cell membrane wherein the extracellular ligand-binding domain of the appended polypeptide is displayed on the cell surface, the transmembrane domain of the appended polypeptide spans cell membrane, and the signaling transducing domain of the appended polypeptide is in the cytoplasmic portion of the cell. In one embodiment, the signal peptide is the signal peptide from human CD8α. In one embodiment, the signal peptide is a functional fragment of the CD8α signal peptide. A functional fragment is defined as a fragment of at least 10 amino acids of the CD8α signal peptide that directs the appended polypeptide to the cell membrane and/or cell surface. Examples of functional fragments of the human CD8α signal peptide include the amino acid sequences MALPVTALLLPLALLLHAA (SEQ ID NO:18), MALPVTALLLP (SEQ ID NO:19), PVTALLPLALL (SEQ ID NO:20), and LLLPLALLLHAARP (SEQ ID NO:21).

Typically, the extracellular ligand-binding domain is linked to the signaling transducing domain of the chimeric antigen receptor (CAR) by a transmembrane domain (Tm). The transmembrane domain traverses the cell membrane, anchors the CAR to the T cell surface, and connects the extracellular ligand-binding domain to the signaling transducing domain, impacting the expression of the CAR on the T cell surface.

The distinguishing feature of the transmembrane domain in the present disclosure is the ability to be expressed at the surface of an immune cell to direct an immune cell response against a pre-defined target cell. The transmembrane domain can be derived from natural or synthetic sources. Alternatively, the transmembrane domain of the present disclosure may be derived from any membrane-bound or transmembrane protein.

Non-limiting examples of transmembrane polypeptides of the present disclosure include the subunits of the T-cell receptor such as α, β, γ, or ζ, polypeptides, constituting the CD3 complex, IL-2 receptor p55 (α chain), p75 (β chain or γ chain), and subunit chains of the Fc receptors, in particular the FcγIII or CD proteins. Alternatively, the transmembrane domain can be synthetic and comprise predominantly hydrophobic amino acid residues (e.g., leucine and valine). In one embodiment, the transmembrane domain is derived from the T-cell surface glycoprotein CD8 alpha chain isoform 1 precursor (NP_001139345.1) selected from CD8α, and CD28.

The transmembrane domain can further comprise a hinge region between extracellular ligand-binding domain and said transmembrane domain. The term “hinge region” generally means any oligo- or polypeptide that functions to link the transmembrane domain to the extracellular ligand-binding domain. In particular, hinge region is used to provide more flexibility and accessibility for the extracellular ligand binding domain. A hinge region may comprise up to 300 amino acids, preferably 10 to 100 amino acids and most preferably 25 to 50 amino acids. Hinge region may be derived from all or parts of naturally-occurring molecules such as CD28, 4-1BB (CD137), OX-40 (CD134), CD3ζ, the T cell receptor α or β chain, CD45, CD4, CD5, CD8, CD8α, CD9, CD16, CD22, CD33, CD37, CD64, CD80, CD86, ICOS, CD154 or from all or parts of an antibody constant region. Alternatively, the hinge region may be a synthetic sequence that corresponds to a naturally-occurring hinge sequence or the hinge region may be an entirely synthetic hinge sequence. In one embodiment, the hinge domain comprises a part of human CD8α, FcγRIIIα receptor, or IgG1, and referred to in this specification as, and have at least 80%, 90%, 95%, 97%, or 99% sequence identity with these polypeptides.

A chimeric antigen receptor (CAR) of the present disclosure comprises a signal transducing domain or intracellular signaling domain of a CAR which is responsible for intracellular signaling following the binding of the extracellular ligand binding domain to the target resulting in the activation of the immune cell and immune response. In other words, the signal transducing domain is responsible for the activation of at least one of the normal effector functions of the immune cell in which the CAR is expressed. For example, the effector function of a T cell can be a cytolytic activity or helper T cell activity, including the secretion of cytokines. Thus, the term “signal transducing domain” refers to the portion of a protein which transduces the effector signal function signal and directs the cell to perform a specialized function.

Examples of signal transducing domains for use in a CAR can be the cytoplasmic sequences of the T cell receptor and co-receptors that act in concert to initiate signal transduction following antigen receptor engagement, as well as any derivate or variant of these sequences and any synthetic sequence that has the same functional capability. Signal transduction domain comprises two distinct classes of cytoplasmic signaling sequence, those that initiate antigen-dependent primary activation, and those that act in an antigen-independent manner to provide a secondary or co-stimulatory signal. Primary cytoplasmic signaling sequence can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Non-limiting examples of ITAM that can be used in the present disclosure can include those derived from TCRζ, FcRγ, FcRβ, FcRε, CD3γ, CD3δ, CD3ε, CDS, CD22, CD79a, CD79b and CD66d. In some embodiments, the signaling transducing domain of the CAR can comprise the CD3ζ signaling domain with an amino acid sequence of at least 80%, 90%, 95%, 97%, or 99% sequence identity thereto.

In addition, the CAR-T cells of the present disclosure may further comprise one or more suicide gene therapy systems. Suitable suicide gene therapy systems known in the art include, but are not limited to, several herpes simplex virus thymidine kinase (HSVtk)/ganciclovir (GCV) or inducible caspase 9 proteins. In one embodiment, the suicide gene is a chimeric CD34/thymidine kinase.

Fratricide Resistance.

T cells disclosed herein may be deficient in an antigen to which the chimeric antigen receptor specifically binds and are therefore fratricide-resistant. In some embodiments, the antigen of the T cell is modified such that the chimeric antigen receptor no longer specifically binds the modified antigen. For example, the epitope of the antigen recognized by the chimeric antigen receptor may be modified by one or more amino acid changes (e.g., substitutions or deletions) or the epitope may be deleted from the antigen. In other embodiments, expression of the antigen is reduced in the T cell by at least 50%, at least 60%, at least 70%, at least 80%, at least 90% or more. Methods for decreasing the expression of a protein are known in the art and include, but are not limited to, modifying or replacing the promoter operably linked to the nucleic acid sequence encoding the protein. In still other embodiments, the T cell is modified such that the antigen is not expressed, e.g., by deletion or disruption of the gene encoding the antigen. In each of the above embodiments, the T cell may be deficient in one or preferably all the antigens to which the chimeric antigen receptor specifically binds. Methods for genetically modifying a T cell to be deficient in an antigen are well known in art, and non-limiting examples are provided above. In an exemplary embodiment, CRISPR/cas9 gene editing can be used to modify a T cell to be deficient in an antigen, for example as described below. Alternatively, TALENs may be used to edit genes.

In certain circumstances, an T cell may be selected for deficiency in the antigen to which the chimeric antigen receptor specifically binds. Certain T cells will produce and display less of a given surface protein; instead if deleting or non-functionalizing the antigen that will be the target of the T-CAR, the T cell can be selected for deficiency in the antigen, and the population of antigen-deficient cells expanded for transduction of the CAR. Such a cell would also be fratricide-resistant.

Avoidance of Alloreactivity.

CAR-T and other CAR-bearing immune effector cells encompassed by the present disclosure may further be deficient in endogenous T cell receptor (TCR) signaling as a result of deleting a part of the T Cell Receptor (TCR)-CD3 complex. In various embodiments it may be desirable to eliminate or suppress endogenous TCR signaling in CAR-bearing immune effector cells disclosed herein. For example, decreasing or eliminating endogenous TCR signaling in CAR-T cells may prevent or reduce graft versus host disease (GvHD) when allogenic T cells are used to produce the CAR-T cells. Methods for eliminating or suppressing endogenous TCR signaling are known in the art and include, but are not limited to, deleting a part of the TCR-CD3 receptor complex, e.g., the TCR receptor alpha chain (TRAC), the TCR receptor beta chain (TRBC), CD3ε CD3γ CD3δ, and/or CD3ζ. Deleting a part of the TCR receptor complex may block TCR mediated signaling and may thus permit the safe use of allogeneic T cells as the source of CAR-T cells without inducing life-threatening GvHD.

CAR Antigens.

Suitable antigens to be genome-edited in the T cells disclosed herein, and to be recognized by the CARs of CAR-T cells disclosed herein, include antigens specific to hematologic malignancies. These can include T cell-specific antigens and/or antigens that are not specific to T cells. The antigen may be specifically bound by the chimeric antigen receptor of a CAR-T cell, and the antigen for which the T-CARs cell is deficient, is an antigen expressed on a malignant T cell, preferably an antigen that is overexpressed on malignant T cell (i.e., a T cell derived from a T-cell malignancy) in comparison to a nonmalignant T cell. Examples of such antigens include CD2, CD3ε, CD4, CD5, CD7, TRAC, and TCRβ.

T-cell malignancies comprise malignancies derived from T-cell precursors, mature T cells, or natural killer cells. Examples of T-cell malignancies include T-cell acute lymphoblastic leukemia/lymphoma (T-ALL), T-cell large granular lymphocyte (LGL) leukemia, human T-cell leukemia virus type 1-positive (HTLV-1+) adult T-cell leukemia/lymphoma (ATL), T-cell prolymphocytic leukemia (T-PLL), and various peripheral T-cell lymphomas (PTCLs), including but not limited to angioimmunoblastic T-cell lymphoma (AITL), ALK-positive anaplastic large cell lymphoma, and ALK-negative anaplastic large cell lymphoma.

Suitable CAR antigens can also include antigens found on the surface of a multiple myeloma cell, i.e., a malignant plasma cell, such as BCMA, CS1, CD38, and CD19. Alternatively, the CAR may be designed to express the extracellular portion of the APRIL protein, the ligand for BCMA and TACI, effectively co-targeting both BCMA and TACI for the treatment of multiple myeloma, B cell lymphoma, B-cell acute lymphoblastic leukemia (B-ALL) and myeloid leukemia.

Additional examples of suitable antigens to be genome-edited in the T cells disclosed herein, and to be recognized by the CARs of the CAR-T cells disclosed herein, are given below in Tables 2-4. These include CD2, CD3ε, CD4, CD5, CD7, TRAC, TCRβ, CS1, CD38.

Suicide Genes.

Alternatively, or in addition, genome-edited T cells may further comprise one or more suicide genes. As used herein, “suicide gene” refers to a nucleic acid sequence introduced to a CAR-T cell by standard methods known in the art that, when activated, results in the death of the CAR-T cell. Suicide genes may facilitate effective tracking and elimination of the T cells in vivo if required. Facilitated killing by activating the suicide gene may occur by methods known in the art. Suitable suicide gene therapy systems known in the art include, but are not limited to, various the herpes simplex virus thymidine kinase (HSVtk)/ganciclovir (GCV) suicide gene therapy systems or inducible caspase 9 protein. In an exemplary embodiment, a suicide gene is a CD34/thymidine kinase chimeric suicide gene.

### Methods of CAR and CAR-T Construction

A “chimeric antigen receptor (CAR),” as used herein and generally used in the art, refers to a recombinant fusion protein that has an antigen-specific extracellular domain (antigen recognition domain) coupled to an intracellular domain (signaling domain) that directs the cell to perform a specialized function upon binding of an antigen to the extracellular domain. Chimeric antigen receptors are distinguished from other antigen binding agents by their ability to both bind MHC-independent antigen and transduce activation signals via their intracellular domain.

Methods for CAR design, delivery and expression, and the manufacturing of clinical-grade CAR-T cell populations are known in the art. See, for example, Lee et al., Clin. Cancer Res., 2012, 18(1 0): 2780-90. An engineered chimeric antigen receptor polynucleotide that encodes for a CAR comprises: a signal peptide, an antigen recognition domain, at least one co-stimulatory domain, and a signalling domain.

The antigen-specific extracellular domain of a chimeric antigen receptor recognizes and specifically binds an antigen, typically a surface-expressed antigen of a malignancy. An “antigen-specific extracellular domain” (or, equivalently, “antigen-binding domain”) specifically binds an antigen when, for example, it binds the antigen with an affinity constant or affinity of interaction (KD) between about 0.1 pM to about 10 μM, preferably about 0.1 pM to about 1 μM, more preferably about 0.1 pM to about 100 nM. Methods for determining the affinity of interaction are known in the art. An antigen-specific extracellular domain suitable for use in a CAR of the present disclosure may be any antigen-binding polypeptide, a wide variety of which are known in the art. In some instances, the antigen-binding domain is a single chain Fv (scFv). Other antibody based recognition domains (cAb VHH (camelid antibody variable domains) and humanized versions thereof, lgNAR VH (shark antibody variable domains) and humanized versions thereof, sdAb VH (single domain antibody variable domains) and “camelized” antibody variable domains are suitable for use. In some instances, T-cell receptor (TCR) based recognition domains such as single chain TCR (scTv, single chain two-domain TCR containing VαVβ) are also suitable for use.

A chimeric antigen receptor of the present disclosure also comprises an “intracellular domain” that provides an intracellular signal to the T cell upon antigen binding to the antigen-specific extracellular domain. The intracellular signaling domain of a chimeric antigen receptor of the present disclosure is responsible for activation of at least one of the effector functions of the T cell in which the chimeric receptor is expressed. The term “effector function” refers to a specialized function of a differentiated cell, such as an T cell. An effector function of an T cell, for example, may be NK transactivation, T cell activation and differentiation, B cell activation, dendritic cell activation and cross-presentation activity, and macrophage activation. Thus, the term “intracellular domain” refers to the portion of a CAR that transduces the effector function signal upon binding of an antigen to the extracellular domain and directs the T cell to perform a specialized function. Non-limiting examples of suitable intracellular domains include the zeta chain of the T-cell receptor or any of its homologs (e.g., eta, delta, gamma, or epsilon), MB 1 chain, 829, Fe Rill, Fe R1, and combinations of signaling molecules, such as CD3ζ and CD28, CD27, 4-1 BB, DAP-1 0, OX40, and combinations thereof, as well as other similar molecules and fragments. Intracellular signaling portions of other members of the families of activating proteins may be used, such as FcγRIII and FcεRI. While usually the entire intracellular domain will be employed, in many cases it will not be necessary to use the entire intracellular polypeptide. To the extent that a truncated portion of the intracellular signaling domain may find use, such truncated portion may be used in place of the intact chain as long as it still transduces the effector function signal. The term intracellular domain is thus meant to include any truncated portion of the intracellular domain sufficient to transduce the effector function signal.

Typically, the antigen-specific extracellular domain is linked to the intracellular domain of the chimeric antigen receptor by a “transmembrane domain.” A transmembrane domain traverses the cell membrane, anchors the CAR to the T cell surface, and connects the extracellular domain to the intracellular signaling domain, thus impacting expression of the CAR on the T cell surface. Chimeric antigen receptors may also further comprise one or more costimulatory domain and/or one or more spacer. A “costimulatory domain” is derived from the intracellular signaling domains of costimulatory proteins that enhance cytokine production, proliferation, cytotoxicity, and/or persistence in vivo. A “spacer” connects (i) the antigen-specific extracellular domain to the transmembrane domain, (ii) the transmembrane domain to a costimulatory domain, (iii) a costimulatory domain to the intracellular domain, and/or (iv) the transmembrane domain to the intracellular domain. For example, inclusion of a spacer domain between the antigen-specific extracellular domain and the transmembrane domain may affect flexibility of the antigen-binding domain and thereby CAR function. Suitable transmembrane domains, costimulatory domains, and spacers are known in the art.

### Mono CAR-T Cells

In certain embodiments, the disclosure provides an engineered T cell comprising a single CAR, that specifically binds an antigen or cell surface protein, wherein the T cell is optionally deficient in that antigen or cell surface protein (e.g., CD7CARTΔCD7 cell). In non-limiting examples, the deficiency in the antigen or cell surface protein resulted from (a) modification of antigen or cell surface protein expressed by the T cell such that the chimeric antigen receptors no longer specifically binds the modified antigen or cell surface protein (e.g., the epitope of the one or more antigens recognized by the chimeric antigen receptor may be modified by one or more amino acid changes (e.g., substitutions or deletions) or the epitope may be deleted from the antigen), (b) modification of the T cell such that expression of antigen or cell surface protein is reduced in the T cell by at least 50%, at least 60%, at least 70%, at least 80%, at least 90% or more, or (c) modification of the T cell such that antigen or cell surface protein is not expressed (e.g., by deletion or disruption of the gene encoding antigen or cell surface protein). In each of the above embodiments, the CAR-T cell may be deficient in one or preferably all the antigens or cell surface proteins to which the chimeric antigen receptor specifically binds. The methods to genetically modify a T cell to be deficient in one or more antigens or cell surface proteins are well known in art and non-limiting examples are provided herein. In embodiments described below, the CRISPR-Cas9 system is used to modify a T cell to be deficient in one or more antigens. Any of these may be accomplished by the methods disclosed herein. In further embodiments, the T cell comprises a suicide gene.

For example, the CAR for a CD7 specific CAR-T cell may be generated by cloning a commercially synthesized anti-CD7 single chain variable fragment (scFv) into a 3rd generation CAR backbone with CD28 and/or 4-1BB internal signaling domains. An extracellular hCD34 domain may be added after a P2A peptide to enable both detection of CAR following viral transduction and purification using anti-hCD34 magnetic beads. A similar method may be followed for making CARs specific for other malignant T cell antigens.

CAR-T cells encompassed by the present disclosure may further be deficient in endogenous T cell receptor (TCR) signaling as a result of deleting a part of the T Cell Receptor (TCR)-CD3 complex. In various embodiments it may be desirable to eliminate or suppress endogenous TCR signaling in CAR-T cells disclosed herein. For example, decreasing or eliminating endogenous TCR signaling in CAR-T cells may prevent or reduce graft versus host disease (GvHD) when allogenic T cells are used to produce the CAR-T cells. Methods for eliminating or suppressing endogenous TCR signaling are known in the art and include, but are not limited to, deleting a part of the TCR-CD3 receptor complex, e.g., the TCR receptor alpha chain (TRAC), the TCR receptor beta chain (TCRβ) or subtypes thereof, TCRδ, TCRγ, CD3ε, CD3γ, and/or CD3δ. Deleting a part of the TCR receptor complex may block TCR mediated signaling and may thus permit the safe use of allogeneic T cells as the source of CAR-T cells without inducing life-threatening GvHD.

In addition, the CAR-T cells encompassed by the present disclosure may further comprise one or more suicide genes as described herein.

In a similar manner, other mono-CAR-T cells may be constructed and are given below in Table 1.

TABLE 1
Mono CARs and CAR-Ts

| | Antigen | |
|---|---|---|
| | Target of | Antigen |
| | CAR-T | Deletion/ |
| Example | cells | Suppression |
| M1 | APRIL | — |
| M2 | APRIL | APRIL |
| M3 | APRIL | APRIL + |
| | | TRAC |
| M4 | APRIL | APRIL + |
| | | CD3ε |
| M5 | APRIL | CD3ε |
| M6 | BCMA | — |
| M7 | CD117 | — |
| M8 | CD117 | CD117 |
| M9 | CD123 | — |
| M10 | CD123 | CD123 |
| M11 | CD135 | — |
| M12 | CD135 | CD135 |
| M13 | CD138 | — |
| M14 | CD19 | — |
| M15 | CD1a | — |
| M16 | CD1a | CD3ε |
| M17 | CD1a | TRAC |
| M18 | CD1a | CD1a + |
| | | TRAC |
| M19 | CD1a | CD1a + CD3ε |
| M20 | CD2 | — |
| M21 | CD2 | CD2 |
| M22 | CD2 | CD2 + TRAC |
| M23 | CD2 | CD2 + CD3ε |
| M24 | CD20 | |
| M25 | CD21 | |
| M26 | CD22 | |
| M27 | CD23 | |
| M28 | CD3 | — |
| M29 | CD3 | CD3ε |
| M30 | CD3 | CD3ε + |
| | | TRAC |
| M31 | CD33 | — |
| M32 | CD33 | CD33 |
| M33 | CD371 | — |
| M34 | CD371 | CD371 |
| M35 | CD38 | — |
| M36 | CD38 | CD38 |
| M37 | CD4 | — |
| M38 | CD4 | CD4 |
| M39 | CD4 | CD4 + TRAC |
| M40 | CD4 | CD4 + CD3ε |
| M41 | CD5 | — |
| M42 | CD5 | CD5 |
| M43 | CD5 | CD5 + TRAC |
| M44 | CD5 | CD5 + CD3ε |
| M45 | CD56 | — |
| M46 | CD56 | CD56 |
| M47 | CD56 | CD56 + |
| | | TRAC |
| M48 | CD56 | CD56 + CD3ε |
| M49 | CD56 | CD3ε |
| M50 | CD56 | TRAC |
| M51 | CD7 | — |
| M52 | CD7 | CD7 |
| M53 | CD7 | CD7 + TRAC |
| M54 | CD7 | CD7 + CD3ε |
| M55 | CD79A | — |
| M56 | CD79B | — |
| M57 | CS1 | — |
| M58 | CS1 | CS1 |
| M59 | Tim-3 | — |
| M60 | Tim-3 | Tim-3 |
| M61 | Tim-3 | Tim-3 + |
| | | TRAC |
| M62 | Tim-3 | TRAC |
| M63 | Tim-3 | CD3ε |
| M64 | Tim-3 | Tim-3 + |
| | | CD3ε |

Disclosed below in Table 2 are embodiments of CAR amino acid sequences that can be expressed on the surface of a genome-edited CAR-T cell derived from a cytotoxic T cell, a memory T cell, or a gamma delta (γδ) T cell.

TABLE 2
Amino Acid Sequences of Mono Chimeric Antigen Receptors (CARs).

| Mono CAR | | |
|---|---|---|
| Constructs | SEQ ID NO: | Amino acid sequence |
| CD7-CAR-4- | SEQ ID | MALPVTALLLPLALLLHAARPDIQMTQTTSSLSASLGDRVT |
| 1BB_CD34 | NO: 1 | ISCSASQGISNYLNWYQQKPDGTVKLLIYYTSSLHSGVPSRF |
| | | SGSGSGTDYSLTISNLEPEDIATYYCQQYSKLPYTFGGGTKL |
| | | EIKRGGGGSGGGGSGGGGSGGGGSEVQLVESGGGLVKPG |
| | | GSLKLSCAASGLTFSSYAMSWVRQTPEKRLEWVASISSGGF |
| | | TYYPDSVKGRFTISRDNARNILYLQMSSLRSEDTAMYYCA |
| | | RDEVRGYLDVWGAGTTVTVSPRASTTTPAPRPPTPAPTIAS |
| | | QPLSLRPEACRPAAGGAVHTRGLDFACDFWVLVVVGGVL |
| | | ACYSLLVTVAFIIFWVKRGRKKLLYIFKQPFMRPVQTTQEE |
| | | DGCSCRFPEEEEGGCELRVKFSRSADAPAYKQGQNQLYNE |
| | | LNLGRREEYDVLDKRRGRDPEMGGKPRRKNPQEGLYNEL |
| | | QKDKMAEAYSEIGMKGERRRGKGHDGLYQGLSTATKDTY |
| | | DALHMQALPPRRTDGSGATNFSLLKQAGDVEENPGPVSEA |
| | | MPRGWTALCLLSLLPSGFMSLDNNGTATPELPTQGTFSNVS |
| | | TNVSYQETTTPSTLGSTSLHPVSQHGNEATTNITETTVKFTS |
| | | TSVITSVYGNTNSSVQSQTSVISTVFTTPANVSTPETTLKPSL |
| | | SPGNVSDLSTTSTSLATSPTKPYTSSSPILSDIKAEIKCSGIRE |
| | | VKLTQGICLEQNKTSSCAEFKKDRGEGLARVLCGEEQADA |
| | | DAGAQVCSLLLAQSEVRPQCLLLVLANRTEISSKLQLMKK |
| | | HQSDLKKLGILDFTEQDVASHQSYSQKTLIALVTSGALLAV |
| | | LGITGYFLMNRRSWSPI |
| CD7-CAR-4- | SEQ ID | MALPVTALLLPLALLLHAARPDIQMTQTTSSLSASLGDRVT |
| 1BB_CD34_TK | NO: 2 | ISCSASQGISNYLNWYQQKPDGTVKLLIYYTSSLHSGVPSRF |
| | | SGSGSGTDYSLTISNLEPEDIATYYCQQYSKLPYTFGGGTKL |
| | | EIKRGGGGSGGGGSGGGGSGGGGSEVQLVESGGGLVKPG |
| | | GSLKLSCAASGLTFSSYAMSWVRQTPEKRLEWVASISSGGF |
| | | TYYPDSVKGRFTISRDNARNILYLQMSSLRSEDTAMYYCA |
| | | RDEVRGYLDVWGAGTTVTVSPRASTTTPAPRPPTPAPTIAS |
| | | QPLSLRPEACRPAAGGAVHTRGLDFACDFWVLVVVGGVL |
| | | ACYSLLVTVAFIIFWVKRGRKKLLYIFKQPFMRPVQTTQEE |
| | | DGCSCRFPEEEEGGCELRVKFSRSADAPAYKQGQNQLYNE |
| | | LNLGRREEYDVLDKRRGRDPEMGGKPRRKNPQEGLYNEL |
| | | QKDKMAEAYSEIGMKGERRRGKGHDGLYQGLSTATKDTY |
| | | DALHMQALPPRRTDGSGATNFSLLKQAGDVEENPGPVSEA |
| | | MPRGWTALCLLSLLPSGFMSLDNNGTATPELPTQGTFSNVS |
| | | TNVSYQETTTPSTLGSTSLHPVSQHGNEATTNITETTVKFTS |
| | | TSVITSVYGNTNSSVQSQTSVISTVFTTPANVSTPETTLKPSL |
| | | SPGNVSDLSTTSTSLATSPTKPYTSSSPILSDIKAEIKCSGIRE |
| | | VKLTQGICLEQNKTSSCAEFKKDRGEGLARVLCGEEQADA |
| | | DAGAQVCSLLLAQSEVRPQCLLLVLANRTEISSKLQLMKK |
| | | HQSDLKKLGILDFTEQDVASHQSYSQKTLIALVTSGALLAV |
| | | LGITGYFLMNRRSWSPTGEGGGGGDLGGVKLPHLFGKRLV |
| | | EARMASYPCHQHASAFDQAARSRGHSNRRTALRPRRQQE |
| | | ATEVRLEQKMPTLLRVYIDGPHGMGKTTTTQLLVALGSRD |
| | | DIVYVPEPMTYWQVLGASETIANIYTTQHRLDQGEISAGDA |
| | | AVVMTSAQITMGMPYAVTDAVLAPHVGGEAGSSHAPPPA |
| | | LTLLLDRHPIAVMLCYPAARYLMGSMTPQAVLAFVALIPPT |
| | | LPGTNIVLGALPEDRHIDRLAKRQRPGERLDLAMLAAIRRV |
| | | YGLLANTVRYLQGGGSWWEDWGQLSGTAVPPQGAEPQS |
| | | NAGPRPHIGDTLFTLFRAPELLAPNGDLYNVFAWALDVLA |
| | | KRLRPMHVFILDYDQSPAGCRDALLQLTSGMVQTHVTTPG |
| | | SIPTICDLARTFAREMGEAN |
| CD7-CAR- | SEQ ID | MALPVTALLLPLALLLHAARPDIQMTQTTSSLSASLGDRVT |
| CD28_CD34 | NO: 3 | ISCSASQGISNYLNWYQQKPDGTVKLLIYYTSSLHSGVPSRF |
| | | SGSGSGTDYSLTISNLEPEDIATYYCQQYSKLPYTFGGGTKL |
| | | EIKRGGGGSGGGGSGGGGSGGGGSEVQLVESGGGLVKPG |
| | | GSLKLSCAASGLTFSSYAMSWVRQTPEKRLEWVASISSGGF |
| | | TYYPDSVKGRFTISRDNARNILYLQMSSLRSEDTAMYYCA |
| | | RDEVRGYLDVWGAGTTVTVSPRASTTTPAPRPPTPAPTIAS |
| | | QPLSLRPEACRPAAGGAVHTRGLDFACDFWVLVVVGGVL |
| | | ACYSLLVTVAFIIFWVRSKRSRLLHSDYMNMTPRRPGPTRK |
| | | HYQPYAPPRDFAAYRSRVKFSRSADAPAYKQGQNQLYNE |
| | | LNLGRREEYDVLDKRRGRDPEMGGKPRRKNPQEGLYNEL |
| | | QKDKMAEAYSEIGMKGERRRGKGHDGLYQGLSTATKDTY |
| | | DALHMQALPPRRTDGSGATNFSLLKQAGDVEENPGPVSEA |
| | | MPRGWTALCLLSLLPSGFMSLDNNGTATPELPTQGTFSNVS |
| | | TNVSYQETTTPSTLGSTSLHPVSQHGNEATTNITETTVKFTS |
| | | TSVITSVYGNTNSSVQSQTSVISTVFTTPANVSTPETTLKPSL |
| | | SPGNVSDLSTTSTSLATSPTKPYTSSSPILSDIKAEIKCSGIRE |
| | | VKLTQGICLEQNKTSSCAEFKKDRGEGLARVLCGEEQADA |
| | | DAGAQVCSLLLAQSEVRPQCLLLVLANRTEISSKLQLMKK |
| | | HQSDLKKLGILDFTEQDVASHQSYSQKTLIALVTSGALLAV |
| | | LGITGYFLMNRRSWSPI |
| CD7-CAR- | SEQ ID | MALPVTALLLPLALLLHAARPDIQMTQTTSSLSASLGDRVT |
| CD28_CD34_TK | NO: 4 | ISCSASQGISNYLNWYQQKPDGTVKLLIYYTSSLHSGVPSRF |
| | | SGSGSGTDYSLTISNLEPEDIATYYCQQYSKLPYTFGGGTKL |
| | | EIKRGGGGSGGGGSGGGGSGGGGSEVQLVESGGGLVKPG |
| | | GSLKLSCAASGLTFSSYAMSWVRQTPEKRLEWVASISSGGF |
| | | TYYPDSVKGRFTISRDNARNILYLQMSSLRSEDTAMYYCA |
| | | RDEVRGYLDVWGAGTTVTVSPRASTTTPAPRPPTPAPTIAS |
| | | QPLSLRPEACRPAAGGAVHTRGLDFACDFWVLVVVGGVL |
| | | ACYSLLVTVAFIIFWVRSKRSRLLHSDYMNMTPRRPGPTRK |
| | | HYQPYAPPRDFAAYRSRVKFSRSADAPAYKQGQNQLYNE |
| | | LNLGRREEYDVLDKRRGRDPEMGGKPRRKNPQEGLYNEL |
| | | QKDKMAEAYSEIGMKGERRRGKGHDGLYQGLSTATKDTY |
| | | DALHMQALPPRRTDGSGATNFSLLKQAGDVEENPGPVSEA |
| | | MPRGWTALCLLSLLPSGFMSLDNNGTATPELPTQGTFSNVS |
| | | TNVSYQETTTPSTLGSTSLHPVSQHGNEATTNITETTVKFTS |
| | | TSVITSVYGNTNSSVQSQTSVISTVFTTPANVSTPETTLKPSL |
| | | SPGNVSDLSTTSTSLATSPTKPYTSSSPILSDIKAEIKCSGIRE |
| | | VKLTQGICLEQNKTSSCAEFKKDRGEGLARVLCGEEQADA |
| | | DAGAQVCSLLLAQSEVRPQCLLLVLANRTEISSKLQLMKK |
| | | HQSDLKKLGILDFTEQDVASHQSYSQKTLIALVTSGALLAV |
| | | LGITGYFLMNRRSWSPTGEGGGGGDLGGVKLPHLFGKRLV |
| | | EARMASYPCHQHASAFDQAARSRGHSNRRTALRPRRQQE |
| | | ATEVRLEQKMPTLLRVYIDGPHGMGKTTTTQLLVALGSRD |
| | | DIVYVPEPMTYWQVLGASETIANIYTTQHRLDQGEISAGDA |
| | | AVVMTSAQITMGMPYAVTDAVLAPHVGGEAGSSHAPPPA |
| | | LTLLLDRHPIAVMLCYPAARYLMGSMTPQAVLAFVALIPPT |
| | | LPGTNIVLGALPEDRHIDRLAKRQRPGERLDLAMLAAIRRV |
| | | YGLLANTVRYLQGGGSWWEDWGQLSGTAVPPQGAEPQS |
| | | NAGPRPHIGDTLFTLFRAPELLAPNGDLYNVFAWALDVLA |
| | | KRLRPMHVFILDYDQSPAGCRDALLQLTSGMVQTHVTTPG |
| | | SIPTICDLARTFAREMGEAN |
| CD79B-CAR- | SEQ ID | MALPVTALLLPLALLLHAARPGSDIQLTQSPSSLSASVGDR |
| CD28_CD34 | NO: 5 | VTITCKASQSVDYEGDSFLNWYQQKPGKAPKLLIYAASNL |
| | | ESGVPSRFSGSGSGTDFTLTISSLQPEDFATYYCQQSNEDPL |
| | | TFGQGTKVEIKRGGGGSGGGGSGGGGSGGGGSGGGGSEV |
| | | QLVESGGGLVQPGGSLRLSCAASGYTFSSYWIEWVRQAPG |
| | | KGLEWIGEILPGGGDTNYNEIFKGRATFSADTSKNTAYLQM |
| | | NSLRAEDTAVYYCTRRVPIRLDYWGQGTLVTVSSPRASTT |
| | | TPAPRPPTPAPTIASQPLSLRPEACRPAAGGAVHTRGLDFAC |
| | | DFWVLVVVGGVLACYSLLVTVAFIIFWVRSKRSRLLHSDY |
| | | MNMTPRRPGPTRKHYQPYAPPRDFAAYRSRVKFSRSADAP |
| | | AYKQGQNQLYNELNLGRREEYDVLDKRRGRDPEMGGKPR |
| | | RKNPQEGLYNELQKDKMAEAYSEIGMKGERRRGKGHDGL |
| | | YQGLSTATKDTYDALHMQALPPRRTDGSGATNFSLLKQAG |
| | | DVEENPGPVSEAMPRGWTALCLLSLLPSGFMSLDNNGTAT |
| | | PELPTQGTFSNVSTNVSYQETTTPSTLGSTSLHPVSQHGNEA |
| | | TTNITETTVKFTSTSVITSVYGNTNSSVQSQTSVISTVFTTPA |
| | | NVSTPETTLKPSLSPGNVSDLSTTSTSLATSPTKPYTSSSPILS |
| | | DIKAEIKCSGIREVKLTQGICLEQNKTSSCAEFKKDRGEGLA |
| | | RVLCGEEQADADAGAQVCSLLLAQSEVRPQCLLLVLANRT |
| | | EISSKLQLMKKHQSDLKKLGILDFTEQDVASHQSYSQKTLI |
| | | ALVTSGALLAVLGITGYFLMNRRSWSPTGEGGGGGFKRDL |
| | | GGVKLPHLFGKRLVEARMASYPCHQHASAFDQAARSRGH |
| | | SNRRTALRPRRQQEATEVRLEQKMPTLLRVYIDGPHGMGK |
| | | TTTTQLLVALGSRDDIVYVPEPMTYWQVLGASETIANIYTT |
| | | QHRLDQGEISAGDAAVVMTSAQITMGMPYAVTDAVLAPH |
| | | VGGEAGSSHAPPPALTLLLDRHPIAVMLCYPAARYLMGSM |
| | | TPQAVLAFVALIPPTLPGTNIVLGALPEDRHIDRLAKRQRPG |
| | | ERLDLAMLAAIRRVYGLLANTVRYLQGGGSWWEDWGQL |
| | | SGTAVPPQGAEPQSNAGPRPHIGDTLFTLFRAPELLAPNGD |
| | | LYNVFAWALDVLAKRLRPMHVFILDYDQSPAGCRDALLQ |
| | | LTSGMVQTHVTTPGSIPTICDLARTFAREMGEAN |
| CD2-CAR- | SEQ ID | MALPVTALLLPLALLLHAARPDIVMTQAAPSVPVTPGESVS |
| CD28_CD34 | NO: 6 | ISCRSSKTLLHSNGNTYLYWFLQRPGQSPQVLIYRMSNLAS |
| | | GVPNRFSGSGSETTFTLRISRVEAEDVGIYYCMQHLEYPYT |
| | | FGGGTKLEIERGGGGSGGGGSGGGGSGGGGSEVQLEESGA |
| | | ELVRPGTSVKLSCKASGYTFTSYWMHWIKQRPEQGLEWIG |
| | | RIDPYDSETHYNEKFKDKAILSVDKSSSTAYIQLSSLTSDDS |
| | | AVYYCSRRDAKYDGYALDYWGQGTSVTVSSPRASTTTPA |
| | | PRPPTPAPTIASQPLSLRPEACRPAAGGAVHTRGLDFACDF |
| | | WVLVVVGGVLACYSLLVTVAFIIFWVRSKRSRLLHSDYMN |
| | | MTPRRPGPTRKHYQPYAPPRDFAAYRSRVKFSRSADAPAY |
| | | KQGQNQLYNELNLGRREEYDVLDKRRGRDPEMGGKPRRK |
| | | NPQEGLYNELQKDKMAEAYSEIGMKGERRRGKGHDGLYQ |
| | | GLSTATKDTYDALHMQALPPRRTDGSGATNFSLLKQAGDV |
| | | EENPGPVSEAMPRGWTALCLLSLLPSGFMSLDNNGTATPEL |
| | | PTQGTFSNVSTNVSYQETTTPSTLGSTSLHPVSQHGNEATT |
| | | NITETTVKFTSTSVITSVYGNTNSSVQSQTSVISTVFTTPANV |
| | | STPETTLKPSLSPGNVSDLSTTSTSLATSPTKPYTSSSPILSDI |
| | | KAEIKCSGIREVKLTQGICLEQNKTSSCAEFKKDRGEGLAR |
| | | VLCGEEQADADAGAQVCSLLLAQSEVRPQCLLLVLANRTE |
| | | ISSKLQLMKKHQSDLKKLGILDFTEQDVASHQSYSQKTLIA |
| | | LVTSGALLAVLGITGYFLMNRRSWSPI |
| CD2-CAR-4- | SEQ ID | MALPVTALLLPLALLLHAARPDIVMTQAAPSVPVTPGESVS |
| 1BB_CD34 | NO: 7 | ISCRSSKTLLHSNGNTYLYWFLQRPGQSPQVLIYRMSNLAS |
| | | GVPNRFSGSGSETTFTLRISRVEAEDVGIYYCMQHLEYPYT |
| | | FGGGTKLEIERGGGGSGGGGSGGGGSGGGGSEVQLEESGA |
| | | ELVRPGTSVKLSCKASGYTFTSYWMHWIKQRPEQGLEWIG |
| | | RIDPYDSETHYNEKFKDKAILSVDKSSSTAYIQLSSLTSDDS |
| | | AVYYCSRRDAKYDGYALDYWGQGTSVTVSSPRASTTTPA |
| | | PRPPTPAPTIASQPLSLRPEACRPAAGGAVHTRGLDFACDF |
| | | WVLVVVGGVLACYSLLVTVAFIIFWVKRGRKKLLYIFKQP |
| | | FMRPVQTTQEEDGCSCRFPEEEEGGCELRVKFSRSADAPAY |
| | | KQGQNQLYNELNLGRREEYDVLDKRRGRDPEMGGKPRRK |
| | | NPQEGLYNELQKDKMAEAYSEIGMKGERRRGKGHDGLYQ |
| | | GLSTATKDTYDALHMQALPPRRTDGSGATNFSLLKQAGDV |
| | | EENPGPVSEAMPRGWTALCLLSLLPSGFMSLDNNGTATPEL |
| | | PTQGTFSNVSTNVSYQETTTPSTLGSTSLHPVSQHGNEATT |
| | | NITETTVKFTSTSVITSVYGNTNSSVQSQTSVISTVFTTPANV |
| | | STPETTLKPSLSPGNVSDLSTTSTSLATSPTKPYTSSSPILSDI |
| | | KAEIKCSGIREVKLTQGICLEQNKTSSCAEFKKDRGEGLAR |
| | | VLCGEEQADADAGAQVCSLLLAQSEVRPQCLLLVLANRTE |
| | | ISSKLQLMKKHQSDLKKLGILDFTEQDVASHQSYSQKTLIA |
| | | LVTSGALLAVLGITGYFLMNRRSWSPI |
| CD3-CD28-CD34 | SEQ ID | MALPVTALLLPLALLLHAARPGSQVQLQQSGAELARPGAS |
| | NO: 8 | VKMSCKASGYTFTRYTMHWVKQRPGQGLEWIGYINPSRG |
| | | YTNYNQKFKDKATLTTDKSSSTAYMQLSSLTSEDSAVYYC |
| | | ARYYDDHYCLDYWGQGTTLTVSSGGGGSGGGGSGGGGS |
| | | GGGGSQIVLTQSPAIMSASPGEKVTMTCSASSSVSYMNWY |
| | | QQKSGTSPKRWIYDTSKLASGVPAHFRGSGSGTSYSLTISG |
| | | MEAEDAATYYCQQWSSNPFTFGSGTKLEINRPRASTTTPAP |
| | | RPPTPAPTIASQPLSLRPEACRPAAGGAVHTRGLDFACDFW |
| | | VLVVVGGVLACYSLLVTVAFIIFWVRSKRSRLLHSDYMNM |
| | | TPRRPGPTRKHYQPYAPPRDFAAYRSRVKFSRSADAPAYK |
| | | QGQNQLYNELNLGRREEYDVLDKRRGRDPEMGGKPRRKN |
| | | PQEGLYNELQKDKMAEAYSEIGMKGERRRGKGHDGLYQG |
| | | LSTATKDTYDALHMQALPPRRTDGSGATNFSLLKQAGDVE |
| | | ENPGPVSEAMPRGWTALCLLSLLPSGFMSLDNNGTATPELP |
| | | TQGTFSNVSTNVSYQETTTPSTLGSTSLHPVSQHGNEATTNI |
| | | TETTVKFTSTSVITSVYGNTNSSVQSQTSVISTVFTTPANVST |
| | | PETTLKPSLSPGNVSDLSTTSTSLATSPTKPYTSSSPILSDIKA |
| | | EIKCSGIREVKLTQGICLEQNKTSSCAEFKKDRGEGLARVL |
| | | CGEEQADADAGAQVCSLLLAQSEVRPQCLLLVLANRTEISS |
| | | KLQLMKKHQSDLKKLGILDFTEQDVASHQSYSQKTLIALV |
| | | TSGALLAVLGITGYFLMNRRSWSPI |

### Tandem CAR-T Cells

A tandem CAR-T cell (tCAR-T), is a T cell with a single chimeric antigen polypeptide comprising two distinct extracellular ligand-binding (antigen/protein recognition) domains capable of interacting with two different cell surface molecules (e.g., antigen/protein), wherein the extracellular ligand-binding domains are linked together by one or more flexible linkers and share one or more costimulatory domains, wherein the binding of the first or second extracellular ligand-binding domain will signal through one or more the costimulatory domains(s) and a signaling transducing domain.

In certain embodiments, the T cell is deficient in one or more antigens or cell surface proteins (e.g., CD7 and CD2 for a CD7*CD2-tCARΔCD7ΔCD2 cell, or CD2 for a CD3*CD2-tCARΔCD3ΔCD2 cell). In non-limiting examples, the deficiency in the antigen(s) or cell surface protein(s) resulted from (a) modification of antigen or cell surface protein expressed by the T cell such that the chimeric antigen receptor no longer specifically binds the modified antigen(s) or cell surface protein(s) (e.g., the epitope of the one or more antigens recognized by the chimeric antigen receptor may be modified by one or more amino acid changes (e.g., substitutions or deletions) or the epitope may be deleted from the antigen), (b) modification of the T cell such that expression of antigen(s) or cell surface protein(s) is/are reduced in the T cell by at least 50%, at least 60%, at least 70%, at least 80%, at least 90% or more, or (c) modification of the T cell such that antigen(s) or cell surface protein(s) is/are not expressed (e.g., by deletion or disruption of the gene encoding antigen or cell surface protein). In each of the above embodiments, the CAR-T cell may be deficient in one or preferably all the antigens or cell surface proteins to which the chimeric antigen receptor specifically binds. The methods to genetically modify a T cell to be deficient in one or more antigens or cell surface proteins are well known in art and non-limiting examples are provided herein. In embodiments described below, the CRISPR-Cas9 system is used to modify a T cell to be deficient in one or more antigen(s) or cell surface protein(s). Any of these may be accomplished by the methods disclosed herein. In further embodiments, the T cell comprises a suicide gene.

A tCAR for a genome-edited, tandem CAR-T cell, i.e., CD2*CD3-tCARTΔCD2ΔCD3ε, may be generated by cloning a commercially synthesized anti-CD2 single chain variable fragment (scFv) and an anti-CD3 single chain variable fragment (scFv), separated by a peptide linker, into a lentiviral vector containing, e.g., a 2^{nd} or 3^{rd} generation CAR backbone with CD28 and/or 4-1BB internal signaling domains. An extracellular hCD34 domain may be added after a P2A peptide to enable both detection of CAR following viral transduction and purification using anti-hCD34 magnetic beads; alternatively, other markers are available, and other methods for generating bicistronic constructs are available. A similar method may be followed for making tCARs specific for other malignant T cell antigens.

Tandem CARs may have different linker structures, i.e., be linear or hairpin, and the hairpin linker may optionally comprise a (Cys=Cys) double-stranded bond (DSB).

A linear tandem CAR-T cell comprises a chimeric antigen receptor (CAR) polypeptide comprising a first signal peptide, a first extracellular ligand-binding domain, a second extracellular ligand-binding domain, a hinge region, a transmembrane domain, one or more co-stimulatory domains, and a signaling transducing domain, wherein the first extracellular ligand-binding antigen recognition domain and the second extracellular ligand-binding antigen recognition domain have affinities for different cell surface molecules, i.e., antigens on a cancer cell, for example, a malignant T cell, B cell, or plasma cell; and wherein the linear tandem CAR-T cell possesses one or more genetic modifications, deletions, or disruptions resulting in reduced expression of the cell surface molecules in the linear tandem CAR-T cell.

In another embodiment, the signal peptide is the signal peptide from human CD8α.

In a third embodiment, the first extracellular ligand-binding domain comprises a single chain antibody fragment (scFv), comprising the light (V_{L}) and the heavy (V_{H}) variable fragment, designated V_{H}1 and V_{L}1 and joined by a linker (e.g., GGGGS). In some embodiments, this linker peptide is repeated 2, 3, 4, 5 or 6 times. In some embodiments, the first antigen recognition domain can be selected from: 1) V_{H}1—(GGGGS)₃₋₄ (SEQ ID NO:414)—V_{L}1 or 2) V_{L}1—(GGGGS)₃₋₄ (SEQ ID NO:414)—V_{H}1.

In some embodiments, the second extracellular ligand-binding domain comprises a single chain antibody fragment (scFv), comprising the light (V_{L}) and the heavy (V_{H}) variable fragment, designated V_{H}2 and V_{L}2 and joined by a linker (e.g., GGGGS). In some embodiments, this linker peptide is repeated 2, 3, 4, 5 or 6 times. In some embodiments, the first antigen recognition domain can be selected from: 1) V_{H}2—(GGGGS)₃₋₄ (SEQ ID NO:414)—V_{L}2 or 2) V_{L}2—(GGGGS)₃₋₄ (SEQ ID NO:414)—V_{H}2.

In further embodiments, the first antigen recognition domain and second antigen recognition domain are connected by a short linker peptide of 5 amino acids (GGGGS). In some embodiments, this linker peptide is repeated 2, 3, 4, 5 or 6 times.

Tandem CAR Constructs

In one embodiment, the first extracellular ligand-binding domain antigen recognition comprises a single chain antibody fragment (scFv), comprising the heavy (V_{H}) and the light (V_{L}) variable fragment, designated V_{H}1 and V_{L}1, and joined by a linker (e.g., GGGGS), targets a cell surface molecule, i.e., an antigen expressed on a malignant T cell.

In certain embodiments, the heavy (V_{H}) and the light (V_{L}) variable fragment, designated V_{H}1 and V_{L}1, targeting an antigen expressed on a malignant T cell is selected from BCMA, CS1, CD38, CD138, CD19, CD33, CD123, CD371, CD117, CD135, Tim-3, CD5, CD7, CD2, CD4, CD3, CD79A, CD79B, APRIL, CD56, and CD1a.

In certain embodiments, the second extracellular ligand-binding domain antigen recognition comprises a single chain antibody fragment (scFv), comprising the heavy (V_{H}) and the light (V_{L}) variable fragment, designated V_{H}2 and V_{L}2, and joined by a linker (e.g., GGGGS), and targets a cell surface molecule, i.e., an antigen, expressed on a malignant cell.

In certain embodiments, the heavy (V_{H}) and the light (V_{L}) variable fragments, designated V_{H}2 and V_{L}2, targeting an antigen expressed on a malignant cell is selected from BCMA, CS1, CD38, CD138, CD19, CD33, CD123, CD371, CD117, CD135, Tim-3, CD5, CD7, CD2, CD4, CD3, CD79A, CD79B, APRIL, CD56, and CD1a and differs from the variable heavy (V_{H}1) and light sequences (V_{L}1) of the first extracellular ligand-binding domain of the CAR molecule.

Additional examples of tandem CARs are given below in Table 3.

TABLE 3
Tandem CARs and CAR-Ts

| Example | Antigen Target CAR-T cell | Antigen Deletion/Suppression |
|---|---|---|
| T1 | APRIL × BCMA | — |
| T2 | APRIL × CD19 | — |
| T3 | APRIL × CD38 | — |
| T4 | APRIL × CD38 | CD38 |
| T5 | APRIL × CS1 | — |
| T6 | APRIL × CS1 | CS1 |
| T7 | BCMA × CD19 | — |
| T8 | BCMA × CD38 | — |
| T9 | BCMA × CD38 | CD38 |
| T10 | BCMA × CS1 | — |
| T11 | BCMA × CS1 | CS1 |
| T12 | CD138 × APRIL | |
| T13 | CD138 × BCMA | |
| T14 | CD138 × CD19 | |
| T15 | CD138 × CD38 | |
| T16 | CD138 × CD38 | CD38 |
| T17 | CD138 × CD79A | |
| T18 | CD138 × CD79B | |
| T19 | CD138 × CS1 | |
| T20 | CD138 × CS1 | CS1 |
| T21 | CD19 × CD38 | — |
| T22 | CD19 × CD38 | CD38 |
| T23 | CD2 × CD3ε | — |
| T24 | CD2 × CD3ε | CD2 |
| T25 | CD2 × CD3ε | CD3ε |
| T26 | CD2 × CD3ε | CD2 and CD3ε |
| T27 | CD2 × CD4 | — |
| T28 | CD2 × CD4 | CD2 |
| T29 | CD2 × CD4 | CD4 |
| T30 | CD2 × CD4 | CD2 and CD4 |
| T31 | CD2 × CD4 | CD2 and TRAC |
| T32 | CD2 × CD4 | CD4 and TRAC |
| T33 | CD2 × CD4 | CD2 and CD4 and TRAC |
| T34 | CD2 × CD5 | — |
| T35 | CD2 × CD5 | CD2 |
| T36 | CD2 × CD5 | CD5 |
| T37 | CD2 × CD5 | CD2 and CD5 |
| T38 | CD2 × CD5 | CD2 and TRAC |
| T39 | CD2 × CD5 | CD5 and TRAC |
| T40 | CD2 × CD5 | CD2 and CD5 and TRAC |
| T41 | CD2 × CD7 | — |
| T42 | CD2 × CD7 | CD2 |
| T43 | CD2 × CD7 | CD7 |
| T44 | CD2 × CD7 | CD2 and CD7 |
| T45 | CD2 × CD7 | CD2 and TRAC |
| T46 | CD2 × CD7 | CD7 and TRAC |
| T47 | CD2 × CD7 | CD2 and CD7 and TRAC |
| T48 | CD3ε × CD4 | — |
| T49 | CD3ε × CD4 | CD3ε |
| T50 | CD3ε × CD4 | CD4 |
| T51 | CD3ε × CD4 | CD3ε and CD4 |
| T52 | CD3ε × CD5 | — |
| T53 | CD3ε × CD5 | CD3ε |
| T54 | CD3ε × CD5 | CD5 |
| T55 | CD3ε × CD5 | CD3ε and CD5 |
| T56 | CD3ε × CD7 | — |
| T57 | CD3ε × CD7 | CD3ε |
| T58 | CD3ε × CD7 | CD7 |
| T59 | CD3ε × CD7 | CD3ε and CD7 |
| T60 | CD4 × CD5 | — |
| T61 | CD4 × CD5 | CD4 |
| T62 | CD4 × CD5 | CD5 |
| T63 | CD4 × CD5 | CD4 and CD5 |
| T64 | CD4 × CD5 | CD4 and TRAC |
| T65 | CD4 × CD5 | CD5 and TRAC |
| T66 | CD4 × CD5 | CD4 and CD5 and TRAC |
| T67 | CD4 × CD7 | — |
| T68 | CD4 × CD7 | CD4 |
| T69 | CD4 × CD7 | CD7 |
| T70 | CD4 × CD7 | CD4 and CD7 |
| T71 | CD4 × CD7 | CD4 and TRAC |
| T72 | CD4 × CD7 | CD4 and TRAC |
| T73 | CD4 × CD7 | CD4 and CD7 and TRAC |
| T74 | CD5 × CD7 | — |
| T75 | CD5 × CD7 | CD5 |
| T76 | CD5 × CD7 | CD7 |
| T77 | CD5 × CD7 | CD5 and CD7 |
| T78 | CD5 × CD7 | CD5 and TRAC |
| T79 | CD5 × CD7 | CD7 and TRAC |
| T80 | CD5 × CD7 | CD5 and CD7 and TRAC |
| T81 | CD79A × APRIL | |
| T82 | CD79A × BCMA | |
| T83 | CD79A × CD19 | |
| T84 | CD79A × CD38 | |
| T85 | CD79A × CD38 | CD38 |
| T86 | CD79A × CD79B | |
| T87 | CD79A × CS1 | |
| T88 | CD79A × CS1 | CS1 |
| T89 | CD79B × APRIL | |
| T90 | CD79B × BCMA | |
| T91 | CD79B × CD19 | |
| T92 | CD79B × CD38 | |
| T93 | CD79B × CD38 | CD38 |
| T94 | CD79B × CD79A | |
| T95 | CD79B × CS1 | |
| T96 | CD79B × CS1 | CS1 |
| T97 | CS1 × CD19 | — |
| T98 | CS1 × CD19 | CS1 |
| T99 | CS1 × CD38 | — |
| T100 | CS1 × CD38 | CS1 |
| T101 | CS1 × CD38 | CD38 |
| T102 | CS1 × CD38 | CS1 and CD38 |
| T103 | TCRβ × CD2 | — |
| T104 | TCRβ × CD2 | TCRβ |
| T105 | TCRβ × CD2 | CD2 |
| T106 | TCRβ × CD2 | TCRβ and CD2 |
| T107 | TCRβ × CD3ε | — |
| T108 | TCRβ × CD3ε | TCRβ |
| T109 | TCRβ × CD3ε | CD3ε |
| T110 | TCRβ × CD3ε | TCRβ and CD3ε |
| T111 | TCRβ × CD4 | — |
| T112 | TCRβ × CD4 | TCRβ |
| T113 | TCRβ × CD4 | CD4 |
| T114 | TCRβ × CD4 | TCRβ and CD4 |
| T115 | TCRβ × CD5 | — |
| T116 | TCRβ × CD5 | TCRβ |
| T117 | TCRβ × CD5 | CD5 |
| T118 | TCRβ × CD5 | TCRβ and CD5 |
| T119 | TCRβ × CD7 | — |
| T120 | TCRβ × CD7 | TCRβ |
| T121 | TCRβ × CD7 | CD7 |
| T122 | TCRβ × CD7 | TCRβ and CD7 |
| T123 | TRAC × CD2 | — |
| T124 | TRAC × CD2 | TRAC |
| T125 | TRAC × CD2 | CD2 |
| T126 | TRAC × CD2 | TRAC and CD2 |
| T127 | TRAC × CD3ε | — |
| T128 | TRAC × CD3ε | TRAC |
| T129 | TRAC × CD3ε | CD3ε |
| T130 | TRAC × CD3ε | TRAC and CD3ε |
| T131 | TRAC × CD4 | — |
| T132 | TRAC × CD4 | TRAC |
| T133 | TRAC × CD4 | CD4 |
| T134 | TRAC × CD4 | TRAC and CD4 |
| T135 | TRAC × CD5 | — |
| T136 | TRAC × CD5 | TRAC |
| T137 | TRAC × CD5 | CD5 |
| T138 | TRAC × CD5 | TRAC and CD5 |
| T139 | TRAC × CD7 | — |
| T140 | TRAC × CD7 | TRAC |
| T141 | TRAC × CD7 | CD7 |
| T142 | TRAC × CD7 | TRAC and CD7 |

For example, provided in Table 4 are linear tandem CAR constructs which may incorporate the V_{H} and V_{L} domains of scFvs targeting any of the antigen pairs provided in Table 3 above.

TABLE 3
Linear Tandem CAR Constructs

| I | II | III | IV | V | VI | VII | VIII |
|---|---|---|---|---|---|---|---|
| CD8a | CD8a | CD8a | CD8a | CD8a | CD8a | CD8a | CD8a |
| V_{H}1 | V_{H}1 | V_{H}1 | V_{H}1 | V_{H}1 | V_{H}1 | V_{H}1 | V_{H}1 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ | (SEQ | (SEQ | (SEQ | (SEQ | (SEQ | (SEQ | (SEQ |
| ID | ID | ID | ID | ID | ID | ID | ID |
| NO: 412) | NO: 412) | NO: 412) | NO: 412) | NO: 412) | NO: 412) | NO: 412) | NO: 412) |
| V_{L}1 | V_{L}1 | V_{L}1 | V_{L}1 | V_{L}1 | V_{L}1 | V_{L}1 | V_{L}1 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ | (SEQ | (SEQ | (SEQ | (SEQ | (SEQ | (SEQ | (SEQ |
| ID | ID | ID | ID | ID | ID | ID | ID |
| NO: 412) | NO: 412) | NO: 412) | NO: 412) | NO: 412) | NO: 412) | NO: 412) | NO: 412) |
| V_{L}2 | V_{L}2 | V_{L}2 | V_{L}2 | V_{L}2 | V_{L}2 | V_{L}2 | V_{L}2 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ | (SEQ | (SEQ | (SEQ | (SEQ | (SEQ | (SEQ | (SEQ |
| ID | ID | ID | ID | ID | ID | ID | ID |
| NO: 414) | NO: 414) | NO: 414) | NO: 414) | NO: 414) | NO: 414) | NO: 414) | NO: 414) |
| V_{H}2 | V_{H}2 | V_{H}2 | V_{H}2 | V_{H}2 | V_{H}2 | V_{H}2 | V_{H}2 |
| CD8 Tm | CD8 Tm | CD8 Tm | CD8 Tm | CD28 Tm | CD28 Tm | CD28 Tm | CD28 Tm |
| 41BB | CD28 | 41BB- | CD28- | 41BB | CD28 | 41BB- | CD28- |
| | | CD28 | 41BB | | | CD28 | 41BB |
| CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ |
| IX | X | XI | XII | XIII | XIV | XV | XVI |
| CD8a | CD8a | CD8a | CD8a | CD8a | CD8a | CD8a | CD8a |
| V_{H}2 | V_{H}2 | V_{H}2 | V_{H}2 | V_{H}2 | V_{H}2 | V_{H}2 | V_{H}2 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ | (SEQ | (SEQ | (SEQ | (SEQ | (SEQ | (SEQ | (SEQ |
| ID | ID | ID | ID | ID | ID | ID | ID |
| NO: 412) | NO: 412) | NO: 412) | NO: 412) | NO: 412) | NO: 412) | NO: 412) | NO: 412) |
| V_{L}2 | V_{L}2 | V_{L}2 | V_{L}2 | V_{L}2 | V_{L}2 | V_{L}2 | V_{L}2 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ | (SEQ | (SEQ | (SEQ | (SEQ | (SEQ | (SEQ | (SEQ |
| ID | ID | ID | ID | ID | ID | ID | ID |
| NO: 412) | NO: 412) | NO: 412) | NO: 412) | NO: 412) | NO: 412) | NO: 412) | NO: 412) |
| V_{L}1 | V_{L}1 | V_{L}1 | V_{L}1 | V_{L}1 | V_{L}1 | V_{L}1 | V_{L}1 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ | (SEQ | (SEQ | (SEQ | (SEQ | (SEQ | (SEQ | (SEQ |
| ID | ID | ID | ID | ID | ID | ID | ID |
| NO: 414) | NO: 414) | NO: 414) | NO: 414) | NO: 414) | NO: 414) | NO: 414) | NO: 414) |
| V_{H}1 | V_{H}1 | V_{H}1 | V_{H}1 | V_{H}1 | V_{H}1 | V_{H}1 | V_{H}1 |
| CD8 Tm | CD8 Tm | CD8 Tm | CD8 Tm | CD28 Tm | CD28 Tm | CD28 Tm | CD28 Tm |
| 41BB | CD28 | 41BB- | CD28- | 41BB | CD28 | 41BB- | CD28- |
| | | CD28 | 41BB | | | CD28 | 41BB |
| CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ |
| XVII | XVIII | XIX | XX | XXI | XXII | XXIII | XIV |
| CD8a | CD8a | CD8a | CD8a | CD8a | CD8a | CD8a | CD8a |
| V_{L}1 | V_{L}1 | V_{L}1 | V_{L}1 | V_{L}1 | V_{L}1 | V_{L}1 | V_{L}1 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ | (SEQ | (SEQ | (SEQ | (SEQ | (SEQ | (SEQ | (SEQ |
| ID | ID | ID | ID | ID | ID | ID | ID |
| NO: 412) | NO: 412) | NO: 412) | NO: 412) | NO: 412) | NO: 412) | NO: 412) | NO: 412) |
| V_{H}1 | V_{H}1 | V_{H}1 | V_{H}1 | V_{H}1 | V_{H}1 | V_{H}1 | V_{H}1 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ | (SEQ | (SEQ | (SEQ | (SEQ | (SEQ | (SEQ | (SEQ |
| ID | ID | ID | ID | ID | ID | ID | ID |
| NO: 412) | NO: 412) | NO: 412) | NO: 412) | NO: 412) | NO: 412) | NO: 412) | NO: 412) |
| V_{H}2 | V_{H}2 | V_{H}2 | V_{H}2 | V_{H}2 | V_{H}2 | V_{H}2 | V_{H}2 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ | (SEQ | (SEQ | (SEQ | (SEQ | (SEQ | (SEQ | (SEQ |
| ID | ID | ID | ID | ID | ID | ID | ID |
| NO: 414) | NO: 414) | NO: 414) | NO: 414) | NO: 414) | NO: 414) | NO: 414) | NO: 414) |
| V_{L}2 | V_{L}2 | V_{L}2 | V_{L}2 | V_{L}2 | V_{L}2 | V_{L}2 | V_{L}2 |
| CD8 Tm | CD8 Tm | CD8 Tm | CD8 Tm | CD28 Tm | CD28 Tm | CD28 Tm | CD28 Tm |
| 41BB | CD28 | 41BB- | CD28- | 41BB | CD28 | 41BB- | CD28- |
| | | CD28 | 41BB | | | CD28 | 41BB |
| CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ |
| XXV | XXVI | XXVII | XXVIII | XIX | XXX | XXXI | XXXII |
| CD8a | CD8a | CD8a | CD8a | CD8a | CD8a | CD8a | CD8a |
| V_{L}2 | V_{L}2 | V_{L}2 | V_{L}2 | V_{L}2 | V_{L}2 | V_{L}2 | V_{L}2 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ | (SEQ | (SEQ | (SEQ | (SEQ | (SEQ | (SEQ | (SEQ |
| ID | ID | ID | ID | ID | ID | ID | ID |
| NO: 412) | NO: 412) | NO: 412) | NO: 412) | NO: 412) | NO: 412) | NO: 412) | NO: 412) |
| V_{H}2 | V_{H}2 | V_{H}2 | V_{H}2 | V_{H}2 | V_{H}2 | V_{H}2 | V_{H}2 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ | (SEQ | (SEQ | (SEQ | (SEQ | (SEQ | (SEQ | (SEQ |
| ID | ID | ID | ID | ID | ID | ID | ID |
| NO: 412) | NO: 412) | NO: 412) | NO: 412) | NO: 412) | NO: 412) | NO: 412) | NO: 412) |
| V_{H}1 | V_{H}1 | V_{H}1 | V_{H}1 | V_{H}1 | V_{H}1 | V_{H}1 | V_{H}1 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ | (SEQ | (SEQ | (SEQ | (SEQ | (SEQ | (SEQ | (SEQ |
| ID | ID | ID | ID | ID | ID | ID | ID |
| NO: 414) | NO: 414) | NO: 414) | NO: 414) | NO: 414) | NO: 414) | NO: 414) | NO: 414) |
| V_{L}1 | V_{L}1 | V_{L}1 | V_{L}1 | V_{L}1 | V_{L}1 | V_{L}1 | V_{L}1 |
| CD8 Tm | CD8 Tm | CD8 Tm | CD8 Tm | CD28 Tm | CD28 Tm | CD28 Tm | CD28 Tm |
| 41BB | CD28 | 41BB- | CD28- | 41BB | CD28 | 41BB- | CD28- |
| | | CD28 | 41BB | | | CD28 | 41BB |
| CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ |

Also provided below in Table 5 are hairpin tandem CAR constructs which may incorporate the V_{H} and V_{L} domains of scFvs targeting any of the antigen pairs provided in Table 3 above.

TABLE 5
Hairpin Tandem CAR Constructs

| I | II | III | IV | V | VI | VII | VIII |
|---|---|---|---|---|---|---|---|
| CD8a | CD8a | CD8a | CD8a | CD8a | CD8a | CD8a | CD8a |
| V_{H}1 | V_{H}1 | V_{H}1 | V_{H}1 | V_{H}1 | V_{H}1 | V_{H}1 | V_{H}1 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ | (SEQ | (SEQ | (SEQ | (SEQ | (SEQ | (SEQ | (SEQ |
| ID | ID | ID | ID | ID | ID | ID | ID |
| NO: 412) | NO: 412) | NO: 412) | NO: 412) | NO: 412) | NO: 412) | NO: 412) | NO: 412) |
| V_{H}2 | V_{H}2 | V_{H}2 | V_{H}2 | V_{H}2 | V_{H}2 | V_{H}2 | V_{H}2 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ | (SEQ | (SEQ | (SEQ | (SEQ | (SEQ | (SEQ | (SEQ |
| ID | ID | ID | ID | ID | ID | ID | ID |
| NO: 412) | NO: 412) | NO: 412) | NO: 412) | NO: 412) | NO: 412) | NO: 412) | NO: 412) |
| V_{L}2 | V_{L}2 | V_{L}2 | V_{L}2 | V_{L}2 | V_{L}2 | V_{L}2 | V_{L}2 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ | (SEQ | (SEQ | (SEQ | (SEQ | (SEQ | (SEQ | (SEQ |
| ID | ID | ID | ID | ID | ID | ID | ID |
| NO: 414) | NO: 414) | NO: 414) | NO: 414) | NO: 414) | NO: 414) | NO: 414) | NO: 414) |
| V_{L}1 | V_{L}1 | V_{L}1 | V_{L}1 | V_{L}1 | V_{L}1 | V_{L}1 | V_{L}1 |
| CD8 Tm | CD8 Tm | CD8 Tm | CD8 Tm | CD28 Tm | CD28 Tm | CD28 Tm | CD28 Tm |
| 41BB | CD28 | 41BB- | CD28- | 41BB | CD28 | 41BB- | CD28- |
| | | CD28 | 41BB | | | CD28 | 41BB |
| CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ |
| IX | X | XI | XII | XIII | XIV | XV | XVI |
| CD8a | CD8a | CD8a | CD8a | CD8a | CD8a | CD8a | CD8a |
| V_{H}2 | V_{H}2 | V_{H}2 | V_{H}2 | V_{H}2 | V_{H}2 | V_{H}2 | V_{H}2 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ | (SEQ | (SEQ | (SEQ | (SEQ | (SEQ | (SEQ | (SEQ |
| ID | ID | ID | ID | ID | ID | ID | ID |
| NO: 412) | NO: 412) | NO: 412) | NO: 412) | NO: 412) | NO: 412) | NO: 412) | NO: 412) |
| V_{H}1 | V_{H}1 | V_{H}1 | V_{H}1 | V_{H}1 | V_{H}1 | V_{H}1 | V_{H}1 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ | (SEQ | (SEQ | (SEQ | (SEQ | (SEQ | (SEQ | (SEQ |
| ID | ID | ID | ID | ID | ID | ID | ID |
| NO: 412) | NO: 412) | NO: 412) | NO: 412) | NO: 412) | NO: 412) | NO: 412) | NO: 412) |
| V_{L}1 | V_{L}1 | V_{L}1 | V_{L}1 | V_{L}1 | V_{L}1 | V_{L}1 | V_{L}1 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ | (SEQ | (SEQ | (SEQ | (SEQ | (SEQ | (SEQ | (SEQ |
| ID | ID | ID | ID | ID | ID | ID | ID |
| NO: 414) | NO: 414) | NO: 414) | NO: 414) | NO: 414) | NO: 414) | NO: 414) | NO: 414) |
| V_{L}2 | V_{L}2 | V_{L}2 | V_{L}2 | V_{L}2 | V_{L}2 | V_{L}2 | V_{L}2 |
| CD8 Tm | CD8 Tm | CD8 Tm | CD8 Tm | CD28 Tm | CD28 Tm | CD28 Tm | CD28 Tm |
| 41BB | CD28 | 41BB- | CD28- | 41BB | CD28 | 41BB- | CD28- |
| | | CD28 | 41BB | | | CD28 | 41BB |
| CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ |
| XVII | XVIII | XIX | XX | XXI | XXII | XXIII | XIV |
| CD8a | CD8a | CD8a | CD8a | CD8a | CD8a | CD8a | CD8a |
| V_{L}1 | V_{L}1 | V_{L}1 | V_{L}1 | V_{L}1 | V_{L}1 | V_{L}1 | V_{L}1 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ | (SEQ | (SEQ | (SEQ | (SEQ | (SEQ | (SEQ | (SEQ |
| ID | ID | ID | ID | ID | ID | ID | ID |
| NO: 412) | NO: 412) | NO: 412) | NO: 412) | NO: 412) | NO: 412) | NO: 412) | NO: 412) |
| V_{L}2 | V_{L}2 | V_{L}2 | V_{L}2 | V_{L}2 | V_{L}2 | V_{L}2 | V_{L}2 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ | (SEQ | (SEQ | (SEQ | (SEQ | (SEQ | (SEQ | (SEQ |
| ID | ID | ID | ID | ID | ID | ID | ID |
| NO: 412) | NO: 412) | NO: 412) | NO: 412) | NO: 412) | NO: 412) | NO: 412) | NO: 412) |
| V_{H}2 | V_{H}2 | V_{H}2 | V_{H}2 | V_{H}2 | V_{H}2 | V_{H}2 | V_{H}2 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ | (SEQ | (SEQ | (SEQ | (SEQ | (SEQ | (SEQ | (SEQ |
| ID | ID | ID | ID | ID | ID | ID | ID |
| NO: 414) | NO: 414) | NO: 414) | NO: 414) | NO: 414) | NO: 414) | NO: 414) | NO: 414) |
| V_{H}1 | V_{H}1 | V_{H}1 | V_{H}1 | V_{H}1 | V_{H}1 | V_{H}1 | V_{H}1 |
| CD8 Tm | CD8 Tm | CD8 Tm | CD8 Tm | CD28 Tm | CD28 Tm | CD28 Tm | CD28 Tm |
| 41BB | CD28 | 41BB- | CD28- | 41BB | CD28 | 41BB- | CD28- |
| | | CD28 | 41BB | | | CD28 | 41BB |
| CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ |
| XXV | XXVI | XXVII | XXVIII | XIX | XXX | XXXI | XXXII |
| CD8a | CD8a | CD8a | CD8a | CD8a | CD8a | CD8a | CD8a |
| V_{L}2 | V_{L}2 | V_{L}2 | V_{L}2 | V_{L}2 | V_{L}2 | V_{L}2 | V_{L}2 |
| GGGGS₍₂₋₆₎ | GGGGS₍₂₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ | (SEQ | (SEQ | (SEQ | (SEQ | (SEQ | (SEQ | (SEQ |
| ID | ID | ID | ID | ID | ID | ID | ID |
| NO: 412) | NO: 412) | NO: 412) | NO: 412) | NO: 412) | NO: 412) | NO: 412) | NO: 412) |
| V_{L}1 | V_{L}1 | V_{L}1 | V_{L}1 | V_{L}1 | V_{L}1 | V_{L}1 | V_{L}1 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ | (SEQ | (SEQ | (SEQ | (SEQ | (SEQ | (SEQ | (SEQ |
| ID | ID | ID | ID | ID | ID | ID | ID |
| NO: 412) | NO: 412) | NO: 412) | NO: 412) | NO: 412) | NO: 412) | NO: 412) | NO: 412) |
| V_{H}1 | V_{H}1 | V_{H}1 | V_{H}1 | V_{H}1 | V_{H}1 | V_{H}1 | V_{H}1 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ | (SEQ | (SEQ | (SEQ | (SEQ | (SEQ | (SEQ | (SEQ |
| ID | ID | ID | ID | ID | ID | ID | ID |
| NO: 414) | NO: 414) | NO: 414) | NO: 414) | NO: 414) | NO: 414) | NO: 414) | NO: 414) |
| V_{H}2 | V_{H}2 | V_{H}2 | V_{H}2 | V_{H}2 | V_{H}2 | V_{H}2 | V_{H}2 |
| CD8 Tm | CD8 Tm | CD8 Tm | CD8 Tm | CD28 Tm | CD28 Tm | CD28 Tm | CD28 Tm |
| 41BB | CD28 | 41BB- | CD28- | 41BB | CD28 | 41BB- | CD28- |
| | | CD28 | 41BB | | | CD28 | 41BB |
| CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ |

Also provided in Table 6 below are hairpin tandem CAR constructs which incorporate the V_{H} and V_{L} domains of CD2 and CD3 scFvs.

TABLE 6
Hairpin Tandem CAR Constructs Targeting CD2 and CD3

| Clone 5 | Clone 6 | Clone 7 | Clone 8 | Clone 13 | Clone 14 | Clone 15 | Clone 16 |
|---|---|---|---|---|---|---|---|
| CD8a | CD8a | CD8a | CD8a | CD8a | CD8a | CD8a | CD8a |
| CD3-V_{L} | CD3-V_{L} | CD3-V_{L} | CD3-V_{L} | CD2-V_{L} | CD2-V_{L} | CD3-V_{L} | CD3-V_{L} |
| GGGGS₄ | GGGGS₄ | GGGGS₄ | GGGGS₄ | GGGGS₄ | GGGGS₄ | GGGGS₄ | GGGGS₄ |
| (SEQ ID | (SEQ ID | (SEQ ID | (SEQ ID | (SEQ ID | (SEQ ID | (SEQ ID | (SEQ ID |
| NO: 415) | NO: 415) | NO: 415) | NO: 415) | NO: 415) | NO: 415) | NO: 415) | NO: 415) |
| CD2-V_{L} | CD2-V_{L} | CD2-V_{L} | CD2-V_{L} | CD3-V_{L} | CD3-V_{L} | CD2-V_{L} | CD2-V_{L} |
| (GGGGS)₁₀ | (GGGGS)₄ | (GGGGS)₁₀ | (GGGGS)₄ | (GGGGS)₁₀ | (GGGGS)₄ | (GGGGS)₁₀ | (GGGGS)₄ |
| (SEQ | GGGG | (SEQ | GGGG | (SEQ | GGGG | (SEQ | GGGG |
| ID | P(GGGG | ID | P(GGGG | ID | P(GGGG | ID | P(GGGG |
| NO: 416) | S)₄ (SEQ | NO: 416) | S)₄ (SEQ | NO: 416) | S)₄ (SEQ | NO: 416) | S)₄ (SEQ |
| | ID | | ID | | ID | | ID |
| | NO: 417) | | NO: 417) | | NO: 417) | | NO: 417) |
| CD2-V_{H} | CD2-V_{H} | CD2-V_{H} | CD2-V_{H} | CD3-V_{H} | CD3-V_{H} | CD2-V_{H} | CD2-V_{H} |
| GGGGS₄ | GGGGS₄ | GGGGS₄ | GGGGS₄ | GGGGS₄ | GGGGS₄ | GGGGS₄ | GGGGS₄ |
| (SEQ ID | (SEQ ID | (SEQ ID | (SEQ ID | (SEQ ID | (SEQ ID | (SEQ ID | (SEQ ID |
| NO: 415) | NO: 415) | NO: 415) | NO: 415) | NO: 415) | NO: 415) | NO: 415) | NO: 415) |
| CD3-V_{H} | CD3-V_{H} | CD3-V_{H} | CD3-V_{H} | CD2-V_{H} | CD2-V_{H} | CD3-V_{H} | CD3-V_{H} |
| CD28 | CD28 | CD28 | CD28 | CD28 | CD28 | CD28 | CD28 |
| Tm | Tm | Tm | Tm | Tm | Tm | Tm | Tm |
| CD28 | CD28 | CD28 | CD28 | CD28 | CD28 | CD28 | CD28 |
| CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ |
| P2A | P2A | P2A | P2A | P2A | P2A | P2A | P2A |
| CD34 | CD34 | CD34 | CD34 | CD34 | CD34 | CD34 | CD34 |

Also provided below in Table 7 are hairpin tandem CAR constructs which may incorporate the V_{H} and V_{L} domains of scFvs targeting any of the antigen pairs provided in Table 3 above.

TABLE 7
Hairpin Tandem DSB CAR Constructs with a (Cys═Cys) Double-Stranded Bond (DSB)

| | | | | | | | |
|---|---|---|---|---|---|---|---|
| CD8a | CD8a | CD8a | CD8a | CD8a | CD8a | CD8a | CD8a |
| V_{H}1 | V_{H}1 | V_{H}1 | V_{H}1 | V_{H}1 | V_{H}1 | V_{H}1 | V_{H}1 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ | (SEQ | (SEQ | (SEQ | (SEQ | (SEQ | (SEQ | (SEQ |
| ID | ID | ID | ID | ID | ID | ID | ID |
| NO: 412) | NO: 412) | NO: 412) | NO: 412) | NO: 412) | NO: 412) | NO: 412) | NO: 412) |
| V_{H}2 | V_{H}2 | V_{H}2 | V_{H}2 | V_{H}2 | V_{H}2 | V_{H}2 | V_{H}2 |
| GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ |
| GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ |
| GGGG | GGGG | GGGG | GGGG | GGGG | GGGG | GGGG | GGGG |
| S₍₁₋₂₎ | S₍₁₋₂₎ | S₍₁₋₂₎ | S₍₁₋₂₎ | S₍₁₋₂₎ | S₍₁₋₂₎ | S₍₁₋₂₎ | S₍₁₋₂₎ |
| GGGGP₍₁₎ | GGGGP₍₁₎ | GGGGP₍₁₎ | GGGGP₍₁₎ | GGGGP₍₁₎ | GGGGP₍₁₎ | GGGGP₍₁₎ | GGGGP₍₁₎ |
| GGGG | GGGG | GGGG | GGGG | GGGG | GGGG | GGGG | GGGG |
| S₍₂₋₃₎ | S₍₂₋₃₎ | S₍₂₋₃₎ | S₍₂₋₃₎ | S₍₂₋₃₎ | S₍₂₋₃₎ | S₍₂₋₃₎ | S₍₂₋₃₎ |
| GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ |
| GGGG | GGGG | GGGG | GGGG | GGGG | GGGG | GGGG | GGGG |
| S₍₀₋₁₎ | S₍₀₋₁₎ | S₍₀₋₁₎ | S₍₀₋₁₎ | S₍₀₋₁₎ | S₍₀₋₁₎ | S₍₀₋₁₎ | S₍₀₋₁₎ |
| (SEQ ID | (SEQ ID | (SEQ ID | (SEQ ID | (SEQ ID | (SEQ ID | (SEQ ID | (SEQ ID |
| NO: 413) | NO: 413) | NO: 413) | NO: 413) | NO: 413) | NO: 413) | NO: 413) | NO: 413) |
| V_{L}2 | V_{L}2 | V_{L}2 | V_{L}2 | V_{L}2 | V_{L}2 | V_{L}2 | V_{L}2 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ | (SEQ | (SEQ | (SEQ | (SEQ | (SEQ | (SEQ | (SEQ |
| ID | ID | ID | ID | ID | ID | ID | ID |
| NO: 414) | NO: 414) | NO: 414) | NO: 414) | NO: 414) | NO: 414) | NO: 414) | NO: 414) |
| V_{L}1 | V_{L}1 | V_{L}1 | V_{L}1 | V_{L}1 | V_{L}1 | V_{L}1 | V_{L}1 |
| CD8 Tm | CD8 Tm | CD8 Tm | CD8 Tm | CD28 Tm | CD28 Tm | CD28 Tm | CD28 Tm |
| 41BB | CD28 | 41BB- | CD28- | 41BB | CD28 | 41BB- | CD28- |
| | | CD28 | 41BB | | | CD28 | 41BB |
| CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ |
| CD8a | CD8a | CD8a | CD8a | CD8a | CD8a | CD8a | CD8a |
| V_{H}2 | V_{H}2 | V_{H}2 | V_{H}2 | V_{H}2 | V_{H}2 | V_{H}2 | V_{H}2 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ | (SEQ | (SEQ | (SEQ | (SEQ | (SEQ | (SEQ | (SEQ |
| ID | ID | ID | ID | ID | ID | ID | ID |
| NO: 412) | NO: 412) | NO: 412) | NO: 412) | NO: 412) | NO: 412) | NO: 412) | NO: 412) |
| V_{H}1 | V_{H}1 | V_{H}1 | V_{H}1 | V_{H}1 | V_{H}1 | V_{H}1 | V_{H}1 |
| GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ |
| GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ |
| GGGG | GGGG | GGGG | GGGG | GGGG | GGGG | GGGG | GGGG |
| S₍₁₋₂₎ | S₍₁₋₂₎ | S₍₁₋₂₎ | S₍₁₋₂₎ | S₍₁₋₂₎ | S₍₁₋₂₎ | S₍₁₋₂₎ | S₍₁₋₂₎ |
| GGGGP₍₁₎ | GGGGP₍₁₎ | GGGGP₍₁₎ | GGGGP₍₁₎ | GGGGP₍₁₎ | GGGGP₍₁₎ | GGGGP₍₁₎ | GGGGP₍₁₎ |
| GGGG | GGGG | GGGG | GGGG | GGGG | GGGG | GGGG | GGGG |
| S₍₂₋₃₎ | S₍₂₋₃₎ | S₍₂₋₃₎ | S₍₂₋₃₎ | S₍₂₋₃₎ | S₍₂₋₃₎ | S₍₂₋₃₎ | S₍₂₋₃₎ |
| GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ |
| GGGG | GGGG | GGGG | GGGG | GGGG | GGGG | GGGG | GGGG |
| S₍₀₋₁₎ | S₍₀₋₁₎ | S₍₀₋₁₎ | S₍₀₋₁₎ | S₍₀₋₁₎ | S₍₀₋₁₎ | S₍₀₋₁₎ | S₍₀₋₁₎ |
| (SEQ ID | (SEQ ID | (SEQ ID | (SEQ ID | (SEQ ID | (SEQ ID | (SEQ ID | (SEQ ID |
| NO: 413) | NO: 413) | NO: 413) | NO: 413) | NO: 413) | NO: 413) | NO: 413) | NO: 413) |
| V_{L}1 | V_{L}1 | V_{L}1 | V_{L}1 | V_{L}1 | V_{L}1 | V_{L}1 | V_{L}1 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ | (SEQ | (SEQ | (SEQ | (SEQ | (SEQ | (SEQ | (SEQ |
| ID | ID | ID | ID | ID | ID | ID | ID |
| NO: 414) | NO: 414) | NO: 414) | NO: 414) | NO: 414) | NO: 414) | NO: 414) | NO: 414) |
| V_{L}2 | V_{L}2 | V_{L}2 | V_{L}2 | V_{L}2 | V_{L}2 | V_{L}2 | V_{L}2 |
| CD8 Tm | CD8 Tm | CD8 Tm | CD8 Tm | CD28 Tm | CD28 Tm | CD28 Tm | CD28 Tm |
| 41BB | CD28 | 41BB- | CD28- | 41BB | CD28 | 41BB- | CD28- |
| | | CD28 | 41BB | | | CD28 | 41BB |
| CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ |
| CD8a | CD8a | CD8a | CD8a | CD8a | CD8a | CD8a | CD8a |
| V_{L}1 | V_{L}1 | V_{L}1 | V_{L}1 | V_{L}1 | V_{L}1 | V_{L}1 | V_{L}1 |
| GGGGS₍₂₋₆₎ | GGGGS₍₂₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ | (SEQ | (SEQ | (SEQ | (SEQ | (SEQ | (SEQ | (SEQ |
| ID | ID | ID | ID | ID | ID | ID | ID |
| NO: 412) | NO: 412) | NO: 412) | NO: 412) | NO: 412) | NO: 412) | NO: 412) | NO: 412) |
| V_{L}2 | V_{L}2 | V_{L}2 | V_{L}2 | V_{L}2 | V_{L}2 | V_{L}2 | V_{L}2 |
| GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ |
| GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ |
| GGGG | GGGG | GGGG | GGGG | GGGG | GGGG | GGGG | GGGG |
| S₍₁₋₂₎ | S₍₁₋₂₎ | S₍₁₋₂₎ | S₍₁₋₂₎ | S₍₁₋₂₎ | S₍₁₋₂₎ | S₍₁₋₂₎ | S₍₁₋₂₎ |
| GGGGP₍₁₎ | GGGGP₍₁₎ | GGGGP₍₁₎ | GGGGP₍₁₎ | GGGGP₍₁₎ | GGGGP₍₁₎ | GGGGP₍₁₎ | GGGGP₍₁₎ |
| GGGG | GGGG | GGGG | GGGG | GGGG | GGGG | GGGG | GGGG |
| S₍₂₋₃₎ | S₍₂₋₃₎ | S₍₂₋₃₎ | S₍₂₋₃₎ | S₍₂₋₃₎ | S₍₂₋₃₎ | S₍₂₋₃₎ | S₍₂₋₃₎ |
| GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ |
| GGGG | GGGG | GGGG | GGGG | GGGG | GGGG | GGGG | GGGG |
| S₍₀₋₁₎ | S₍₀₋₁₎ | S₍₀₋₁₎ | S₍₀₋₁₎ | S₍₀₋₁₎ | S₍₀₋₁₎ | S₍₀₋₁₎ | S₍₀₋₁₎ |
| (SEQ ID | (SEQ ID | (SEQ ID | (SEQ ID | (SEQ ID | (SEQ ID | (SEQ ID | (SEQ ID |
| NO: 413) | NO: 413) | NO: 413) | NO: 413) | NO: 413) | NO: 413) | NO: 413) | NO: 413) |
| V_{H}2 | V_{H}2 | V_{H}2 | V_{H}2 | V_{H}2 | V_{H}2 | V_{H}2 | V_{H}2 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ | (SEQ | (SEQ | (SEQ | (SEQ | (SEQ | (SEQ | (SEQ |
| ID | ID | ID | ID | ID | ID | ID | ID |
| NO: 414) | NO: 414) | NO: 414) | NO: 414) | NO: 414) | NO: 414) | NO: 414) | NO: 414) |
| V_{H}1 | V_{H}1 | V_{H}1 | V_{H}1 | V_{H}1 | V_{H}1 | V_{H}1 | V_{H}1 |
| CD8 Tm | CD8 Tm | CD8 Tm | CD8 Tm | CD28 Tm | CD28 Tm | CD28 Tm | CD28 Tm |
| 41BB | CD28 | 41BB- | CD28- | 41BB | CD28 | 41BB- | CD28- |
| | | CD28 | 41BB | | | CD28 | 41BB |
| CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ |
| CD8a | CD8a | CD8a | CD8a | CD8a | CD8a | CD8a | CD8a |
| V_{L}2 | V_{L}2 | V_{L}2 | V_{L}2 | V_{L}2 | V_{L}2 | V_{L}2 | V_{L}2 |
| GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ | GGGGS₍₂₋₆₎ |
| (SEQ | (SEQ | (SEQ | (SEQ | (SEQ | (SEQ | (SEQ | (SEQ |
| ID | ID | ID | ID | ID | ID | ID | ID |
| NO: 412) | NO: 412) | NO: 412) | NO: 412) | NO: 412) | NO: 412) | NO: 412) | NO: 412) |
| V_{L}1 | V_{L}1 | V_{L}1 | V_{L}1 | V_{L}1 | V_{L}1 | V_{L}1 | V_{L}1 |
| GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ | GGGGS₍₀₋₁₎ |
| GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ |
| GGGG | GGGG | GGGG | GGGG | GGGG | GGGG | GGGG | GGGG |
| S₍₁₋₂₎ | S₍₁₋₂₎ | S₍₁₋₂₎ | S₍₁₋₂₎ | S₍₁₋₂₎ | S₍₁₋₂₎ | S₍₁₋₂₎ | S₍₁₋₂₎ |
| GGGGP₍₁₎ | GGGGP₍₁₎ | GGGGP₍₁₎ | GGGGP₍₁₎ | GGGGP₍₁₎ | GGGGP₍₁₎ | GGGGP₍₁₎ | GGGGP₍₁₎ |
| GGGG | GGGG | GGGG | GGGG | GGGG | GGGG | GGGG | GGGG |
| S₍₂₋₃₎ | S₍₂₋₃₎ | S₍₂₋₃₎ | S₍₂₋₃₎ | S₍₂₋₃₎ | S₍₂₋₃₎ | S₍₂₋₃₎ | S₍₂₋₃₎ |
| GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ | GGGGC₍₁₎ |
| GGGG | GGGG | GGGG | GGGG | GGGG | GGGG | GGGG | GGGG |
| S₍₀₋₁₎ | S₍₀₋₁₎ | S₍₀₋₁₎ | S₍₀₋₁₎ | S₍₀₋₁₎ | S₍₀₋₁₎ | S₍₀₋₁₎ | S₍₀₋₁₎ |
| (SEQ ID | (SEQ ID | (SEQ ID | (SEQ ID | (SEQ ID | (SEQ ID | (SEQ ID | (SEQ ID |
| NO: 413) | NO: 413) | NO: 413) | NO: 413) | NO: 413) | NO: 413) | NO: 413) | NO: 413) |
| V_{H}1 | V_{H}1 | V_{H}1 | V_{H}1 | V_{H}1 | V_{H}1 | V_{H}1 | V_{H}1 |
| GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ | GGGGS₍₃₋₄₎ |
| (SEQ | (SEQ | (SEQ | (SEQ | (SEQ | (SEQ | (SEQ | (SEQ |
| ID | ID | ID | ID | ID | ID | ID | ID |
| NO: 414) | NO: 414) | NO: 414) | NO: 414) | NO: 414) | NO: 414) | NO: 414) | NO: 414) |
| V_{H}2 | V_{H}2 | V_{H}2 | V_{H}2 | V_{H}2 | V_{H}2 | V_{H}2 | V_{H}2 |
| CD8 Tm | CD8 Tm | CD8 Tm | CD8 Tm | CD28 Tm | CD28 Tm | CD28 Tm | CD28 Tm |
| 41BB | CD28 | 41BB- | CD28- | 41BB | CD28 | 41BB- | CD28- |
| | | CD28 | 41BB | | | CD28 | 41BB |
| CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ | CD3z₍₁₋₂₎ |

### Dual CAR-T Cells

In certain embodiments, the disclosure provides an engineered T cell with two distinct chimeric antigen receptor polypeptides with affinity to different antigen(s) or cell surface protein(s) expressed within the same effector cell, wherein each CAR functions independently. The CAR may be expressed from single or multiple polynucleotide sequences that specifically bind different antigen(s) or cell surface protein(s), wherein the T cell is deficient in the antigen(s) or cell surface protein(s) to which the CARs bind (e.g., CD7*CD2-dCARΔCD7ΔCD2 cell). In non-limiting examples, the deficiency in the antigen(s) or cell surface protein(s) resulted from (a) modification of antigen or cell surface protein expressed by the T cell such that the chimeric antigen receptor no longer specifically binds the modified antigen(s) or cell surface protein(s) (e.g., the epitope of the one or more antigens recognized by the chimeric antigen receptor may be modified by one or more amino acid changes (e.g., substitutions or deletions) or the epitope may be deleted from the antigen), (b) modification of the T cell such that expression of antigen(s) or cell surface protein(s) is/are reduced in the T cell by at least 50%, at least 60%, at least 70%, at least 80%, at least 90% or more, or (c) modification of the T cell such that antigen(s) or cell surface protein(s) is/are not expressed (e.g., by deletion or disruption of the gene encoding antigen or cell surface protein). In each of the above embodiments, the CAR-T cell may be deficient in one or preferably all the antigens or cell surface proteins to which the chimeric antigen receptor specifically binds. The methods to genetically modify a T cell to be deficient in one or more antigens or cell surface proteins are well known in art and non-limiting examples are provided herein. In embodiments described below, the CRISPR-Cas9 system is used to modify a T cell to be deficient in one or more antigen(s) or cell surface protein(s). Any of these may be accomplished by the methods disclosed herein. In further embodiments, the T cell comprises a suicide gene.

A dCAR for a genome-edited, dual CAR-T cell, i.e., CD2*CD3ε-dCARTΔCD2ΔCD3ε, may be generated by cloning a commercially synthesized anti-CD2 single chain variable fragment into a lentiviral vector containing, e.g., a 2^{nd} or 3^{rd} generation CAR backbone with CD28 and/or 4-1BB internal signaling domains and cloning a commercially synthesized anti-CD3ε single chain variable into the same lentiviral vector containing an additional 2^{nd} or 3^{rd} generation CAR backbone with CD28 and/or 4-1BB internal signaling domains resulting in a plasmid from which the two CAR constructs are expressed from the same vector. An extracellular hCD34 domain may be added after a P2A peptide to enable both detection of CAR following viral transduction and purification using anti-hCD34 magnetic beads. A similar method may be followed for making tCARs specific for other malignant T cell antigens.

In a similar manner, other dual CARs may be constructed and are given below in Tables 3-4.

In one embodiment, a dual CAR-T cell comprises (i) a first chimeric antigen receptor (CAR) polypeptide comprising a first signal peptide, a first antigen recognition domain, a first hinge region, a first transmembrane domain, a first co-stimulatory domain, and a first signaling domain; and (ii) a second chimeric antigen receptor polypeptide comprising a second signaling peptide, a second antigen recognition domain, a second hinge region, a second transmembrane domain, a second co-stimulatory domain, and a second signaling domain; wherein the first antigen recognition domain and the second antigen recognition domain have affinities for different target antigens; and wherein the dual CAR-T cell possesses one or more genetic disruptions resulting in reduced expression of the target antigen in the dual CAR-T cell.

In a second embodiment, the first signal peptide is a CD8a signal sequence.

In a third embodiment, the first antigen recognition domain is fusion protein of the variable regions of immunoglobulin heavy and light chains, designated V_{H}1 and V_{L}1, for the first antigen recognition domain, connected by a short linker peptide of 5 amino acids (GGGGS). In some embodiments, this linker peptide is repeated 3 or 4 times. in some embodiments, the first antigen recognition domain can be selected from V_{H}1—(GGGGS)₃₋₄ (SEQ ID NO:414)—V_{L}1 or V_{L}1—(GGGGS)₃₋₄ (SEQ ID NO:414)—V_{H}1.

In some embodiments, the first hinge region comprises CD8a.

In some embodiments, the first transmembrane domain is CD8 or CD28.

In some embodiments, the first co-stimulatory domain comprises 4-1BB, CD28, or a combination of both, in either order, i.e., 4-1BB-CD28 or CD28-4-1BB.

In some embodiments, the first signaling domain is CD3ζ or a CD3ζ bi-peptide, i.e. CD3ζ-CD3ζ.

In some embodiments, the second signal peptide is a CD8a signal sequence of SEQ NO:1.

In some embodiments, the second antigen recognition domain is fusion protein of the variable regions of immunoglobulin heavy and light chains, designated V_{H}2 and V_{L}2, for the second antigen recognition domain, connected by a short linker peptide of 5 amino acids (GGGGS). In some embodiments, this linker peptide is repeated 3 or 4 times. In some embodiments, the second antigen recognition domain can be selected from V_{H}2—(GGGGS)₃₋₄ (SEQ ID NO:414)—V_{L}2 or V_{L}2—(GGGGS)₃₋₄ (SEQ ID NO:414)—V_{H}2.

In some embodiments, the second hinge region comprises CD8a.

In some embodiments, the second transmembrane domain is CD8 or CD28.

In some embodiments, the second co-stimulatory domain comprises 4-1BB, CD28, or a combination of both, in either order, i.e. 4-1BB-CD28 or CD28-4-1BB.

In some embodiments, the second signaling domain is CD3ζ or a CD3ζ bi-peptide, i.e. CD3ζ-CD3ζ.

In some embodiments, the CAR polypeptide comprises a first antigen recognition domain fusion protein of V_{H}1—(GGGGS)₃₋₄ (SEQ ID NO:414)—V_{L}1 and a second antigen recognition domain fusion protein of V_{H}2—(GGGGS)₃₋₄ (SEQ ID NO:414)—V_{L}2.

In some embodiments, the CAR polypeptide comprises a first antigen recognition domain fusion protein of V_{L}1—(GGGGS)₃₋₄ (SEQ ID NO:414)—V_{H}1 and a second antigen recognition domain fusion protein of V_{L}2—(GGGGS)₃₋₄ (SEQ ID NO:414)—V_{H}2.

In some embodiments, the CAR polypeptide comprises a first antigen recognition domain fusion protein of V_{H}2—(GGGGS)₃₋₄ (SEQ ID NO:414)—V_{L}2 and a second antigen recognition domain fusion protein of V_{H}1—(GGGGS)₃₋₄ (SEQ ID NO:414)—V_{L}1.

In some embodiments, the CAR polypeptide comprises a first antigen recognition domain fusion protein of V_{H}2—(GGGGS)₃₋₄ (SEQ ID NO:414)—V_{H}2 and a second antigen recognition domain fusion protein of V_{L}1—(GGGGS)₃₋₄ (SEQ ID NO:414)—V_{H}1.

In some embodiments, the CAR polypeptide comprises a first antigen recognition domain fusion protein of V_{H}1—(GGGGS)₃₋₄ (SEQ ID NO:414)—V_{L}1 and a second antigen recognition domain fusion protein of V_{L}2—(GGGGS)₃₋₄ (SEQ ID NO:414)—V_{H}2.

In some embodiments, the CAR polypeptide comprises a first antigen recognition domain fusion protein of V_{L}1—(GGGGS)₃₋₄ (SEQ ID NO:414)—V_{H}1 and a second antigen recognition domain fusion protein of V_{H}2—(GGGGS)₃₋₄ (SEQ ID NO:414)—V_{L}2.

In some embodiments, the CAR polypeptide comprises a first antigen recognition domain fusion protein of V_{H}2—(GGGGS)₃₋₄ (SEQ ID NO:414)—V_{L}2 and a second antigen recognition domain fusion protein of V_{L}1—(GGGGS)₃₋₄ (SEQ ID NO:414)—V_{H}1.

In some embodiments, the CAR polypeptide comprises a first antigen recognition domain fusion protein of V_{L}2—(GGGGS)₃₋₄ (SEQ ID NO:414)—V_{H}2 and a second antigen recognition domain fusion protein of V_{H}1—(GGGGS)₃₋₄ (SEQ ID NO:414)—V_{L}1.

In some embodiments, the CAR polypeptide comprises at least one high efficiency cleavage site, wherein the high efficiency cleavage site is selected from P2A, T2A, E2A, and F2A.

In some embodiments, the CAR polypeptide comprises a suicide gene.

In some embodiments, the CAR polypeptide comprises a cytokine.

In some embodiments, the CAR polypeptide comprises a mutant cytokine.

In some embodiments, the CAR polypeptide comprises a cytokine receptor.

In some embodiments, the CAR polypeptide comprises a mutant cytokine receptor.

In some embodiments, the dual CAR-T cell targets two antigens selected from CD5, CD7, CD2, CD4, CD3, CD33, CD123 (IL3RA), CD371 (CLL-1; CLEC12A), CD117 (c-kit), CD135 (FLT3), BCMA, CS1, CD38, CD79A, CD79B, CD138, and CD19, APRIL, and TACI.

Additional examples of dual CARs are given below in Table 8.

TABLE 8
Dual CARs and dCAR-Ts

| | Antigen | Antigen |
|---|---|---|
| Example | Targets of CARs in dCAR-T cell | Deletion/Suppression |
| D1 | APRIL × BCMA | — |
| D2 | APRIL × CD19 | — |
| D3 | APRIL × CD38 | — |
| D4 | APRIL × CD38 | CD38 |
| D5 | APRIL × CS1 | — |
| D6 | APRIL × CS1 | CS1 |
| D7 | BCMA × CD19 | — |
| D8 | BCMA × CD38 | — |
| D9 | BCMA × CD38 | CD38 |
| D10 | BCMA × CS1 | — |
| D11 | BCMA × CS1 | CS1 |
| D12 | CD138 × APRIL | |
| D13 | CD138 × BCMA | |
| D14 | CD138 × CD19 | |
| D15 | CD138 × CD38 | |
| D16 | CD138 × CD38 | CD38 |
| D17 | CD138 × CD79A | |
| D18 | CD138 × CD79B | |
| D19 | CD138 × CS1 | |
| D20 | CD138 × CS1 | CS1 |
| D21 | CD19 × CD38 | — |
| D22 | CD19 × CD38 | CD38 |
| D23 | CD2 × CD3ε | — |
| D24 | CD2 × CD3ε | CD2 |
| D25 | CD2 × CD3ε | CD3ε |
| D26 | CD2 × CD3ε | CD2 and CD3ε |
| D27 | CD2 × CD4 | — |
| D28 | CD2 × CD4 | CD2 |
| D29 | CD2 × CD4 | CD4 |
| D30 | CD2 × CD4 | CD2 and CD4 |
| D31 | CD2 × CD4 | CD2 and TRAC |
| D32 | CD2 × CD4 | CD4 and TRAC |
| D33 | CD2 × CD4 | CD2 and CD4 and TRAC |
| D34 | CD2 × CD5 | — |
| D35 | CD2 × CD5 | CD2 |
| D36 | CD2 × CD5 | CD5 |
| D37 | CD2 × CD5 | CD2 and CD5 |
| D38 | CD2 × CD5 | CD2 and TRAC |
| D39 | CD2 × CD5 | CD5 and TRAC |
| D40 | CD2 × CD5 | CD2 and CD5 and TRAC |
| D41 | CD2 × CD7 | — |
| D42 | CD2 × CD7 | CD2 |
| D43 | CD2 × CD7 | CD7 |
| D44 | CD2 × CD7 | CD2 and CD7 |
| D45 | CD2 × CD7 | CD2 and TRAC |
| D46 | CD2 × CD7 | CD7 and TRAC |
| D47 | CD2 × CD7 | CD2 and CD7 and TRAC |
| D48 | CD3ε × CD4 | — |
| D49 | CD3ε × CD4 | CD3ε |
| D50 | CD3ε × CD4 | CD4 |
| D51 | CD3ε × CD4 | CD3ε and CD4 |
| D52 | CD3ε × CD5 | — |
| D53 | CD3ε × CD5 | CD3ε |
| D54 | CD3ε × CD5 | CD5 |
| D55 | CD3ε × CD5 | CD3ε and CD5 |
| D56 | CD3ε × CD7 | — |
| D57 | CD3ε × CD7 | CD3ε |
| D58 | CD3ε × CD7 | CD7 |
| D59 | CD3ε × CD7 | CD3ε and CD7 |
| D60 | CD4 × CD5 | — |
| D61 | CD4 × CD5 | CD4 |
| D62 | CD4 × CD5 | CD5 |
| D63 | CD4 × CD5 | CD4 and CD5 |
| D64 | CD4 × CD5 | CD4 and TRAC |
| D65 | CD4 × CD5 | CD5 and TRAC |
| D66 | CD4 × CD5 | CD4 and CD5 and TRAC |
| D67 | CD4 × CD7 | — |
| D68 | CD4 × CD7 | CD4 |
| D69 | CD4 × CD7 | CD7 |
| D70 | CD4 × CD7 | CD4 and CD7 |
| D71 | CD4 × CD7 | CD4 and TRAC |
| D72 | CD4 × CD7 | CD7 and TRAC |
| D73 | CD4 × CD7 | CD4 and CD7 and TRAC |
| D74 | CD5 × CD7 | — |
| D75 | CD5 × CD7 | CD5 |
| D76 | CD5 × CD7 | CD7 |
| D77 | CD5 × CD7 | CD5 and CD7 |
| D78 | CD5 × CD7 | CD5 and TRAC |
| D79 | CD5 × CD7 | CD7 and TRAC |
| D80 | CD5 × CD7 | CD5 and CD7 and TRAC |
| D81 | CD79A × APRIL | |
| D82 | CD79A × BCMA | |
| D83 | CD79A × CD19 | |
| D84 | CD79A × CD38 | |
| D85 | CD79A × CD38 | CD38 |
| D86 | CD79A × CD79B | |
| D87 | CD79A × CS1 | |
| D88 | CD79A × CS1 | CS1 |
| D89 | CD79B × APRIL | |
| D90 | CD79B × BCMA | |
| D91 | CD79B × CD19 | |
| D92 | CD79B × CD38 | |
| D93 | CD79B × CD38 | CD38 |
| D94 | CD79B × CD79A | |
| D95 | CD79B × CS1 | |
| D96 | CD79B × CS1 | CS1 |
| D97 | CS1 × CD19 | — |
| D98 | CS1 × CD19 | CS1 |
| D99 | CS1 × CD38 | — |
| D100 | CS1 × CD38 | CS1 |
| D101 | CS1 × CD38 | CD38 |
| D102 | CS1 × CD38 | CS1 and CD38 |
| D103 | TCRβ × CD2 | — |
| D104 | TCRβ × CD2 | TCRβ |
| D105 | TCRβ × CD2 | CD2 |
| D106 | TCRβ × CD2 | TCRβ and CD2 |
| D107 | TCRβ × CD3ε | — |
| D108 | TCRβ × CD3ε | TCRβ |
| D109 | TCRβ × CD3ε | CD3ε |
| D110 | TCRβ × CD3ε | TCRβ and CD3ε |
| D111 | TCRβ × CD4 | — |
| D112 | TCRβ × CD4 | TCRβ |
| D113 | TCRβ × CD4 | CD4 |
| D114 | TCRβ × CD4 | TCRβ and CD4 |
| D115 | TCRβ × CD5 | — |
| D116 | TCRβ × CD5 | TCRβ |
| D117 | TCRβ × CD5 | CD5 |
| D118 | TCRβ × CD5 | TCRβ and CD5 |
| D119 | TCRβ × CD7 | — |
| D120 | TCRβ × CD7 | TCRβ |
| D121 | TCRβ × CD7 | CD7 |
| D122 | TCRβ × CD7 | TCRβ and CD7 |
| D123 | TRAC × CD2 | — |
| D124 | TRAC × CD2 | TRAC |
| D125 | TRAC × CD2 | CD2 |
| D126 | TRAC × CD2 | TRAC and CD2 |
| D127 | TRAC × CD3ε | — |
| D128 | TRAC × CD3ε | TRAC |
| D129 | TRAC × CD3ε | CD3ε |
| D130 | TRAC × CD3ε | TRAC and CD3ε |
| D131 | TRAC × CD4 | — |
| D132 | TRAC × CD4 | TRAC |
| D133 | TRAC × CD4 | CD4 |
| D134 | TRAC × CD4 | TRAC and CD4 |
| D135 | TRAC × CD5 | — |
| D136 | TRAC × CD5 | TRAC |
| D137 | TRAC × CD5 | CD5 |
| D138 | TRAC × CD5 | TRAC and CD5 |
| D139 | TRAC × CD7 | — |
| D140 | TRAC × CD7 | TRAC |
| D141 | TRAC × CD7 | CD7 |
| D142 | TRAC × CD7 | TRAC and CD7 |

In a further aspect, a CAR-T cell control may be created. For example, the control CAR-T cell may include an extracellular domain that binds to an antigen not expressed on a malignant T-cell. For example, if the therapeutic CAR-T cell targets a T-cell antigen such as CD7, or multiple T cell antigens, such as CD2 and CD3, the antigen the control CAR-T cell binds to may be CD19, CD19 is an antigen expressed on B cells but not on T cells, so a CAR-T cell with an extracellular domain adapted to bind to CD19 will not bind to T cells. These CAR-T cells may be used as controls to analyze the binding efficiencies and non-specific binding of CAR-T cells targeted to the cancer of interest and/or recognizing the antigen of interest.

CARs may be further designed as disclosed in WO2018027036A1, optionally employing variations which will be known to those of skill in the art. Lentiviral vectors and cell lines can be obtained, and guide RNAs designed, validated, and synthesized, as disclosed therein as well as by methods known in the art and from commercial sources.

Engineered CARs may be introduced into T cells using retroviruses, which efficiently and stably integrate a nucleic acid sequence encoding the chimeric antigen receptor into the target cell genome. Other methods known in the art include, but are not limited to, lentiviral transduction, transposon-based systems, direct RNA transfection, and CRISPR/Cas systems (e.g., type I, type II, or type Ill systems using a suitable Cas protein such Cas3, Cas4, Cas5, Cas5e (or CasD), Cas6, Cas6e, Cas6f, Cas7, Cas8a1, Cas8a2, Cas8b, Cas8c, Cas9, Cas10, Casl Od, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1 0, Csx16, CsaX, Csx3, Csz1, Csx15, Csf1, Csf2, Csf3, Csf4, and Cu1966, etc.). Zinc finger nucleases (ZFNs) and transcription activator-like effector nucleases (TALENs) may also be used. See, e.g., Shearer R F and Saunders D N, “Experimental design for stable genetic manipulation in mammalian cell lines: lentivirus and alternatives,” Genes Cells 2015 January; 20(1):1-10.

Manipulation of PI3K signaling can be used to prevent altered CAR-T cell differentiation due to constitutive CAR self-signaling and foster long-lived memory T cell development. pharmacologic blockade of PI3K during CAR-T manufacture and ex vivo expansion can abrogate preferential effector T cell development and restore CAR-T effector/memory ratio to that observed in empty vector transduced T cells, which can improve in vivo T cell persistence and therapeutic activity. Inhibition of p110δ PI3K can enhance efficacy and memory in tumor-specific therapeutic CD8 T cells, while inhibition of p110α PI3K can increase cytokine production and antitumor response.

This is proposed to be because the presence of a CAR on a T cell's surface can alter its activation and differentiation, even in the absence of ligand. Constitutive self-signaling through CAR, related to both the scFv framework and the signaling domains, can lead to aberrant T cell behavior, including altered differentiation and decreased survival. This is significant as the effectiveness of CAR-T cells in patients is directly associated with their in vivo longevity. The presence of the CD28 costimulatory domain increased CAR-T cell exhaustion induced by persistent CAR self-signaling; the 4-1BB costimulatory domain had a lesser effect. Furthermore, CD3-zeta significantly enhances the constitutive activation of the PI3K, AKT, mTOR, and glycolysis pathways, and fostered formation of short-lived effector cells over central/stem memory cells. See, e.g., Zhang W. et al., “Modulation of PI3K signaling to improve CAR T cell function,” Oncotarget, 2018 Nov. 9; 9(88): 35807-35808.

### Cytokine/Chemokine/Transcription Factor Gene Deletion or Suppression

In addition to gene-editing the TCR and cell surface proteins and antigens, genes for secretable proteins such as cytokines may be edited by the methods disclosed herein. Chemokines, and transcription factors may be edited prior to activation. Such editing would be done, e.g., to reduce or prevent the development or maintenance of cytokine release syndrome (CRS). CRS is caused by a large, rapid release of cytokines from immune cells in response to immunotherapy (or other immunological stimulus). Modifying, disrupting, or deleting one or more cytokine or chemokine genes can be accomplished using the methods disclosed herein.

Cytokines, chemokines, and transcription factors that can be deleted from immune effector cells as disclosed herein, e.g., using Cas9-CRISPR or by targeted transduction of a CAR into the gene sequence of the cytokine, chemokine, or transcription factor include without limitation the following: XCL1, XCL2, CCL1, CCL2, CCL3, CCL4, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CX3CL1, IL-1α (IL1A), IL-1β (IL1B), IL-1RA, IL-18, IL-2, IL-4, IL-7, IL-9, IL-13, IL-15, IL-3, IL-5, GM-CSF, IL-6, IL-11, G-CSF, IL-12, LIF, OSM, IL-10, IL-20, IL-14, IL-16, IL-17, IFN-α, IFN-β, IFN-γ, CD154, LT-β, TNF-α, TNF-β, 4-1BBL, APRIL, CD70, CD153, CD178, GITRL, LIGHT, OX40L, TALL-1, TRAIL, TWEAK, TRANCE, TGF-β1, TGF-β2, TGF-β3, Epo, Tpo, Flt-3L, SCF, M-CSF, MSP, A2M, ACKR1, ACKR2, ACKR3, ACVR1, ACVR2B, ACVRL1, ADIPOQ, AGER, AGRN, AHR, AIMP1, AREG, BCL6. BMP1, BMP10, BMP15, BMP2, BMP3, BMP4, BMP5, BMP6, BMP7, BMP8A, BMP8B, BMPR2, C10orf99, C1QTNF4, C5, CCL28, CCR1, CCR2, CCR3, CCR5, CCR6, CCR7, CD109, CD27, CD28, CD36, CD4, CD40LG, CD70, CD74, CD8a, CER1, CHRD, CKLF, CLCF1, CMTM1, CMTM2, CMTM3, CMTM4, CMTM5, CMTM6, CMTM7, CMTM8, CNTF, CNTFR, COPS5, CRLF1, CSF1, CSF1R, CSF2, CSF3, CSF3R, CTF1, CX3CR1, CXCL16, CXCL17, CXCR1, CXCR2, CXCR3, CXCR4, CXCR6, EBI3, EDN1, ELANE, ENG, FAM3B, FAM3C, FAM3D, FAS, FASLG, FGF2, FLT3LG, FOXP3, FZD4, GATA3, GBP1, GDF1, GDF10, GDF11, GDF15, GDF2, GDF3, GDF5, GDF6, GDF7, GDF9, GPI, GREM1, GREM2, GRN, HAX1, HFE2, HMGB1, HYAL2, ICAM3, ICOS, IFNA10, IFNA14, IFNA16, IFNA2, IFNA5, IFNA6, IFNA8, IFNAR1, IFNAR2, IFNB1, IFNE, IFNG, IFNGR1, IFNK, IFNL1, IFNL3, IFNW1, IL10RA, IL11RA, IL12A, IL12B, IL12RB1, IL17A, IL17B, IL17C, IL17D, IL17F, IL18BP, IL-19, IL1F10, IL1R1, IL1R2, IL1RAPL1, IL1RL1, IL1RN, IL20RA, IL20RB, IL21, IL22, IL22RA1, IL22RA2, IL23A, IL23R, IL24, IL25, IL26, IL27, IL2RA, IL2RB, IL2RG, IL31, IL31RA, IL32, IL33, IL34, IL36A, IL36B, IL36G, IL36RN, IL37, IL6R, IL6ST, INHA, INHBA, INHBB, INHBC, INHBE, ITGA4, ITGAV, ITGB1, ITGB3, KIT, KITLG, KLHL20, LEFTY1, LEFTY2, LIFR, LTA, LTB, LTBP1, LTBP3, LTBP4, MAF, MIF, MINOS1-, MSTN, NAMPT, NBL1, NDP, NLRP7, NODAL, NOG, NRG1, NRP1, NRP2, OSM, OSMR, PARK7, PDPN, PF4, PF4V1, PGLYRP1, PLP2, PPBP, PXDN, RORC, SCG2, SCGB3A1, SECTM1, SLURP1, SOSTDC1, SP100, SPI1, SPP1, TBX21, TCAP, TGFBR1, TGFBR2, TGFBR3, THBS1, THNSL2, THPO, TIMP1, TNF, TNFRSF11, TNFRSF4, TNFRSF1A, TNFRSF9, TNFRSF10, TNFSF10, TNFSF11, TNFSF12, TNFSF12-, TNFSF13, TNFSF13B, TNFSF14, TNFSF15, TNFSF18, TNFSF4, TNFSF8, TNFSF9, TRIM16, TSLP, TWSG1, TXLNA, VASN, VEGFA, VSTM1, WFIKKN1, WFIKKN2, WNT1, WNT2, WNT5A, WNT7A, and ZFP36.

In some embodiments, the cytokine is chosen from cytokine is chosen from MCP1 (CCL2), MCP-2, GM-CSF, G-CSF, M-CSF, 11-4, and IFNγ.

Transcription factors that can be deleted from immune effector cells as disclosed herein, e.g., using Cas9-CRISPR or by targeted transduction of a CAR into the gene sequence of the transcription factor is chosen from AHR, BCL6, FOXP3, GATA3, MAF, RORC, SPI1, and TBX21

The sequences of these genes are known and available in the art.

### Indications and Standards of Care in CAR-T Therapy

In some embodiment, the genome-edited immune effector cells disclosed herein, and/or generated using the methods disclosed herein, express one or more chimeric antigen receptors (CARs) and can be used as a medicament, i.e., for the treatment of disease. In many embodiments, the cells are CAR-T cells.

Cells disclosed herein, and/or generated using the methods disclosed herein, may be used in immunotherapy and adoptive cell transfer, for the treatment, or the manufacture of a medicament for treatment, of cancers, autoimmune diseases, infectious diseases, and other conditions.

The cancer may be a hematologic malignancy or solid tumor. Hematologic malignancies include leukemias, lymphomas, multiple myeloma, and subtypes thereof. Lymphomas can be classified various ways, often based on the underlying type of malignant cell, including Hodgkin's lymphoma (often cancers of Reed-Sternberg cells, but also sometimes originating in B cells; all other lymphomas are non-Hodgkin's lymphomas), B-cell lymphomas, T-cell lymphomas, mantle cell lymphomas, Burkitt's lymphoma, follicular lymphoma, and others as defined herein and known in the art.

B-cell lymphomas include, but are not limited to, diffuse large B-cell lymphoma (DLBCL), chronic lymphocytic leukemia (CLL)/small lymphocytic lymphoma (SLL), B cell-precursor acute lymphoblastic leukemia (ALL), and others as defined herein and known in the art.

T-cell lymphomas include T-cell acute lymphoblastic leukemia/lymphoma (T-ALL), peripheral T-cell lymphoma (PTCL), T-cell chronic lymphocytic leukemia (T-CLL), Sezary syndrome, and others as defined herein and known in the art.

Leukemias include Acute myeloid (or myelogenous) leukemia (AML), chronic myeloid (or myelogenous) leukemia (CML), acute lymphocytic (or lymphoblastic) leukemia (ALL), chronic lymphocytic leukemia (CLL) hairy cell leukemia (sometimes classified as a lymphoma), and others as defined herein and known in the art.

Plasma cell malignancies include lymphoplasmacytic lymphoma, plasmacytoma, and multiple myeloma.

In some embodiments, the medicament can be used for treating cancer in a patient, particularly for the treatment of solid tumors such as melanomas, neuroblastomas, gliomas or carcinomas such as tumors of the brain, head and neck, breast, lung (e.g., non-small cell lung cancer, NSCLC), reproductive tract (e.g., ovary), upper digestive tract, pancreas, liver, renal system (e.g., kidneys), bladder, prostate and colorectum.

In another embodiment, the medicament can be used for treating cancer in a patient, particularly for the treatment of hematologic malignancies selected from multiple myeloma and acute myeloid leukemia (AML) and for T-cell malignancies selected from T-cell acute lymphoblastic leukemia (T-ALL), non-Hodgkin's lymphoma, and T-cell chronic lymphocytic leukemia (T-CLL).

In some embodiments, the cells may be used in the treatment of autoimmune diseases such as lupus, autoimmune (rheumatoid) arthritis, multiple sclerosis, transplant rejection, Crohn's disease, ulcerative colitis, dermatitis, and the like. In some embodiments, the cells are chimeric autoantibody receptor T-cells, or CAAR-Ts displaying antigens or fragments thereof, instead of antibody fragments; in this version of adoptive cell transfer, the B cells that cause autoimmune diseases will attempt to attack the engineered T cells, which will respond by killing them.

In some embodiments, the cells may be used in the treatment of infectious diseases such as HIV and tuberculosis.

In another embodiment, the CAR-T cells of the present disclosure can undergo robust in vivo T cell expansion and can persist for an extended amount of time.

In some embodiments, the treatment of a patient with CAR-T cells of the present disclosure can be ameliorating, curative or prophylactic. It may be either part of an autologous immunotherapy or part of an allogenic immunotherapy treatment. By autologous, it is meant that cells, cell line or population of cells used for treating patients are originating from said patient or from a Human Leucocyte Antigen (HLA) compatible donor. By allogeneic, is meant that the cells or population of cells used for treating patients are not originating from the patient but from a donor.

The treatment of cancer with CAR-T cells of the present disclosure may be in combination with one or more therapies selected from antibody therapy, chemotherapy, cytokine therapy, dendritic cell therapy, gene therapy, hormone therapy, radiotherapy, laser light therapy, and radiation therapy.

The administration of CAR-T cells or a population of CAR-T cells of the present disclosure of the present disclosure be carried out by aerosol inhalation, injection, ingestion, transfusion, implantation or transplantation. The CAR-T cells compositions described herein, i.e., mono CAR, dual CAR, tandem CARs, may be administered to a patient subcutaneously, intradermally, intratumorally, intranodally, intramedullary, intramuscularly, by intravenous or intralymphatic injection, or intraperitoneally. In one embodiment, the cell compositions of the present disclosure are preferably administered by intravenous injection.

The administration of CAR-T cells or a population of CAR-T cells can consist of the administration of 10⁴-10⁹ cells per kg body weight, preferably 10⁵ to 10⁶ cells/kg body weight including all integer values of cell numbers within those ranges. The CAR-T cells or a population of CAR-T cells can be administrated in one or more doses. In another embodiment, the effective amount of CAR-T cells or a population of CAR-T cells are administrated as a single dose. In another embodiment, the effective amount of cells are administered as more than one dose over a period time. Timing of administration is within the judgment of a health care provider and depends on the clinical condition of the patient. The CAR-T cells or a population of CAR-T cells may be obtained from any source, such as a blood bank or a donor. While the needs of a patient vary, determination of optimal ranges of effective amounts of a given CAR-T cell population(s) for a particular disease or conditions are within the skill of the art. An effective amount means an amount which provides a therapeutic or prophylactic benefit. The dosage administered will be dependent upon the age, health and weight of the patient recipient, type of concurrent treatment, if any, frequency of treatment, and the nature of the effect desired.

In another embodiment, the effective amount of CAR-T cells or a population of CAR-T cells or composition comprising those CAR-T cells are administered parenterally. The administration can be an intravenous administration. The administration of CAR-T cells or a population of CAR-T cells or composition comprising those CAR-T cells can be directly done by injection within a tumor.

In one embodiment of the present disclosure, the CAR-T cells or a population of the CAR-T cells are administered to a patient in conjunction with, e.g., before, simultaneously or following, any number of relevant treatment modalities, including but not limited to, treatment with cytokines, or expression of cytokines from within the CAR-T, that enhance T-cell proliferation and persistence and, include but are not limited to, IL-2, IL-7, and IL-15.

In a second embodiment, the CAR-T cells or a population of CAR-T cells of the present disclosure may be used in combination with agents that inhibit immunosuppressive pathways, including but not limited to, inhibitors of TGFβ, interleukin 10 (IL-10), adenosine, VEGF, indoleamine 2,3 dioxygenase 1 (IDO1), indoleamine 2,3-dioxygenase 2 (IDO2), tryptophan 2-3-dioxygenase (TDO), lactate, hypoxia, arginase, and prostaglandin E2.

In another embodiment, the CAR-T cells or a population of CAR-T cells of the present disclosure may be used in combination with T-cell checkpoint inhibitors, including but not limited to, anti-CTLA4 (Ipilimumab) anti-PD1 (Pembrolizumab, Nivolumab, Cemiplimab), anti-PDL1 (Atezolizumab, Avelumab, Durvalumab), anti-PDL2, anti-BTLA, anti-LAG3, anti-TIM3, anti-VISTA, anti-TIGIT, and anti-MR.

In another embodiment, the CAR-T cells or a population of CAR-T cells of the present disclosure may be used in combination with T cell agonists, including but not limited to, antibodies that stimulate CD28, ICOS, OX-40, CD27, 4-1BB, CD137, GITR, and HVEM

In another embodiment, the CAR-T cells or a population of CAR-T cells of the present disclosure may be used in combination with therapeutic oncolytic viruses, including but not limited to, retroviruses, picornaviruses, rhabdoviruses, paramyxoviruses, reoviruses, parvoviruses, adenoviruses, herpesviruses, and poxviruses.

In another embodiment, the CAR-T cells or a population of CAR-T cells of the present disclosure may be used in combination with immunostimulatory therapies, such as toll-like receptors agonists, including but not limited to, TLR3, TLR4, TLR7 and TLR9 agonists.

In another embodiment, the CAR-T cells or a population of CAR-T cells of the present disclosure may be used in combination with stimulator of interferon gene (STING) agonists, such as cyclic GMP-AMP synthase (cGAS).

Immune effector cell aplasia, particularly T cell aplasia is also a concern after adoptive cell transfer therapy. When the malignancy treated is a T-cell malignancy, and CAR-T cells target a T cell antigen, normal T cells and their precursors expressing the antigen will become depleted, and the immune system will be compromised. Accordingly, methods for managing these side effects are attendant to therapy. Such methods include selecting and retaining non-malignant T cells or precursors, either autologous or allogeneic (optionally engineered not to cause rejection or be rejected), for later expansion and re-infusion into the patient, after CAR-T cells are exhausted or deactivated. Alternatively, CAR-T cells which recognize and kill subsets of TCR-bearing cells, such as normal and malignant TRBC1⁺, but not TRBC2⁺ cells, or alternatively, TRBC2⁺, but not TRBC1⁺ cells, may be used to eradicate a T cell malignancy while preserving sufficient normal T cells to maintain normal immune system function.

### Definitions

As used herein, the terms below have the meanings indicated. Other definitions may occur throughout the specification.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “activation” (and other conjugations thereof) in reference to cells is generally understood to be synonymous with “stimulating” and as used herein refers to treatment of cells that results in expansion of cell populations. In T cells, activation is often accomplished by exposure to CD2 and CD28 (and sometimes CD2 as well) agonists, typically antibodies, optionally coated onto magnetic beads or conjugated to a colloidal polymeric matrix.

The term “antigen” as used herein is a cell surface protein recognized by (i.e., that is the target of) T cell receptor or chimeric antigen receptor. In the classical sense antigens are substances, typically proteins, that are recognized by antibodies, but the definitions overlap insofar as the CAR comprises antibody-derived domains such as light (V_{L}) and heavy (V_{H}) chains recognizing one or more antigen(s).

The term “cancer” refers to a malignancy or abnormal growth of cells in the body. Many different cancers can be characterized or identified by particular cell surface proteins or molecules. Thus, in general terms, cancer in accordance with the present disclosure may refer to any malignancy that may be treated with an immune effector cell, such as a CAR-T cell as described herein, in which the immune effector cell recognizes and binds to the cell surface protein on the cancer cell. As used herein, cancer may refer to a hematologic malignancy, such as multiple myeloma, a T-cell malignancy, or a B cell malignancy. T cell malignancies may include, but are not limited to, T-cell acute lymphoblastic leukemia (T-ALL) or non-Hodgkin's lymphoma. A cancer may also refer to a solid tumor, such as including, but not limited to, cervical cancer, pancreatic cancer, ovarian cancer, mesothelioma, and lung cancer.

A “cell surface protein” as used herein is a protein (or protein complex) expressed by a cell at least in part on the surface of the cell. Examples of cell surface proteins include the TCR (and subunits thereof) and CD7.

A “chimeric antigen receptor” or “CAR” as used herein and generally used in the art, refers to a recombinant fusion protein that has an extracellular ligand-binding domain, a transmembrane domain, and a signaling transducing domain that directs the cell to perform a specialized function upon binding of the extracellular ligand-binding domain to a component present on the target cell. For example, a CAR can have an antibody-based specificity for a desired antigen (e.g., tumor antigen) with a T cell receptor-activating intracellular domain to generate a chimeric protein that exhibits specific anti-target cellular immune activity. First-generation CARs include an extracellular ligand-binding domain and signaling transducing domain, commonly CD3ζ or FcεRIγ. Second generation CARs are built upon first generation CAR constructs by including an intracellular costimulatory domain, commonly 4-1BB or CD28. These costimulatory domains help enhance CAR-T cell cytotoxicity and proliferation compared to first generation CARs. The third generation CARs include multiple costimulatory domains, primarily to increase CAR-T cell proliferation and persistence. Chimeric antigen receptors are distinguished from other antigen binding agents by their ability both to bind MHC-independent antigens and transduce activation signals via their intracellular domain.

A “CAR-bearing immune effector cell” is an immune effector cell which has been transduced with at least one CAR. A “CAR-T cell” is a T cell which has been transduced with at least one CAR; CAR-T cells can be mono, dual, or tandem CAR-T cells. CAR-T cells can be autologous, meaning that they are engineered from a subject's own cells, or allogeneic, meaning that the cells are sourced from a healthy donor, and in many cases, engineered so as not to provoke a host-vs-graft or graft-vs-host reaction. Donor cells may also be sourced from cord blood or generated from induced pluripotent stem cells.

The term dual CAR-T (dCAR-T), means a CAR-T cell that expresses cells two distinct chimeric antigen receptor polypeptides with affinity to different target antigen expressed within the same effector cell, wherein each CAR functions independently. The CAR may be expressed from single or multiple polynucleotide sequences.

The term tandem CAR-T (tCAR-T) means a single chimeric antigen polypeptide containing two distinct antigen recognition domains with affinity to different targets wherein the antigen recognition domain is linked through a peptide linker and share common costimulatory domain(s), wherein the binding of either antigen recognition domain will signal through a common co-stimulatory domains(s) and signaling domain.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “composition” as used herein refers to an immunotherapeutic cell population combination with one or more therapeutically acceptable carriers.

The term “deletion” as used herein in reference to the effect of editing on a gene or its protein product, means alteration or loss of part the sequence of DNA encoding the protein so as to reduce or prevent expression of the protein product. The term “suppression” in the same context means to reduce expression of the protein product; and the term “ablation” in the same context means to prevent expression of the protein product. Deletion encompasses suppression and ablation.

As used herein, to be “deficient,” as in expression of a gene edited target antigen, or in TCR signaling, means to lack sufficient quantity of antigen or signaling to elicit its normal effect. A cell that is “deficient” in CD7, for example, (a “CD7-deficient” cell) could be entirely lacking in CD7, but it also could express such a negligible quantity of CD7 that the CD7 present could not contribute in any meaningful way to fratricide.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “donor template” refers to the reference genomic material that the cell uses as a template to repair the a double-stranded break through the homology-directed repair (HDR) DNA repair pathway. The donor template contains the piece of DNA to be inserted into the genome (containing the gene to be expressed, CAR, or marker) with two homology arms flanking the site of the double-stranded break. In some embodiments, a donor template may be an adeno-associated virus, a single-stranded DNA, or a double-stranded DNA.

The term “exposing to,” as used herein, in the context of bringing compositions of matter (such as antibodies) into intimate contact with other compositions of matter (such as cells), is intended to be synonymous with “incubated with,” and no lengthier period of time in contact is intended by the use of one term instead of the other.

The term “fratricide” as used herein means a process which occurs when a CAR-T cell (or other CAR-bearing immune effector cell) becomes the target of, and is killed by, another CAR-T cell comprising the same chimeric antigen receptor as the target of CAR-T cell, because the targeted cell expresses the antigen specifically recognized by the chimeric antigen receptor on both cells. CAR-T comprising a chimeric antigen receptor which are deficient in an antigen to which the chimeric antigen receptor specifically binds will be “fratricide-resistant.”

The term “genome-edited” or “gene-edited” as used herein means having a gene or portion of the genome added, deleted, or modified (e.g., disrupted) to be non-functional. Thus, in certain embodiments, a “genome-edited T cell” is a T cell that has had a gene such as a CAR recognizing at least one antigen added; and/or has had a gene such as the gene(s) to the antigen(s) that are recognized by the CAR deleted, and/or has had the gene to the TCR or a subunit thereof disrupted.

A “healthy donor,” as used herein, is one who does not have a malignancy (particularly a hematologic malignancy, e.g., a T-cell malignancy).

As used herein, an “immune effector cell” is a leukocyte that can modulate an immune response. Immune effector cells include T cells, B cells, natural killer (NK) cells, iNKT cells (invariant T-cell receptor alpha natural killer T cells), and macrophages. T cell receptor (TCR)-bearing immune effector cells include, of course, T cells, but also cells which have been engineered to express a T cell receptor.

A “malignant B cell” is a B cell derived from a B-cell malignancy. B cell malignancies include, without limitation, (DLBCL), chronic lymphocytic leukemia (CLL)/small lymphocytic lymphoma (SLL), and B cell-precursor acute lymphoblastic leukemia (ALL).

A “malignant plasma cell” is a plasma cell derived from a plasma cell malignancy. The term “plasma-cell malignancy” refers to a malignancy in which abnormal plasma cells are overproduced. Non-limiting examples of plasma cell malignancies include lymphoplasmacytic lymphoma, plasmacytoma, and multiple myeloma.

A “malignant T cell” is a T cell derived from a T-cell malignancy. The term “T-cell malignancy” refers to a broad, highly heterogeneous grouping of malignancies derived from T-cell precursors, mature T cells, or natural killer cells. Non-limiting examples of T-cell malignancies include T-cell acute lymphoblastic leukemia/lymphoma (T-ALL), human T-cell leukemia virus type 1-positive (HTLV-1+) adult T-cell leukemia/lymphoma (ATL), T-cell prolymphocytic leukemia (T-PLL), Adult T-cell lymphoma/leukemia (HTLV-1 associated), Aggressive NK-cell leukemia, Anaplastic large-cell lymphoma (ALCL), ALK positive, Anaplastic large-cell lymphoma (ALCL), ALK negative, Angioimmunoblastic T-cell lymphoma (AITL), Breast implant-associated anaplastic large-cell lymphoma, Chronic lymphoproliferative disorder of NK cells, Extra nodal NK/T-cell lymphoma, nasal type, Enteropathy-type T-cell lymphoma, Follicular T-cell lymphoma, Hepatosplenic T-cell lymphoma, Indolent T-cell lymphoproliferative disorder of the GI tract, Monomorphic epitheliotrophic intestinal T-cell lymphoma, Mycosis fungoides, Nodal peripheral T-cell lymphoma with TFH phenotype, Peripheral T-cell lymphoma (PTCL), NOS, Primary cutaneous α/β T-cell lymphoma, Primary cutaneous CD8+ aggressive epidermotropic cytotoxic T-cell lymphoma, Primary cutaneous acral CD8+ T-cell lymphoma, Primary cutaneous CD4+ small/medium T-cell lymphoproliferative disorders [Primary cutaneous anaplastic large-cell lymphoma (C-ALCL), lymphoid papulosis], Sezary syndrome, Subcutaneous, panniculitis-like T-cell lymphoma, Systemic EBV+ T-cell lymphoma of childhood, and T-cell large granular lymphocytic leukemia (LGL).

The term “patient” is generally synonymous with the term “subject” and includes all mammals including humans.

As used herein, a “secretable protein” is s protein secreted by a cell which has an effect on other cells. By way of example, secretable proteins include ctyokines, chemokines, and transcription factors.

As used herein, “suicide gene” refers to a nucleic acid sequence introduced to a CAR-T cell by standard methods known in the art, that when activated result in the death of the CAR-T cell. If required suicide genes may facilitate the tracking and elimination, i.e., killing, of CAR-T cells in vivo. Facilitated killing of CAR-T cells by activating a suicide gene can be accomplished by standard methods known in the art. Suicide gene systems known in the art include, but are not limited to, several herpes simplex virus thymidine kinase (HSVtk)/ganciclovir (GCV) suicide gene therapy systems and inducible caspase 9 proteins. In one embodiment, the suicide gene is a chimeric CD34/thymidine kinase.

The term “therapeutically acceptable” refers to substances which are suitable for use in contact with the tissues of patients without undue toxicity, irritation, and allergic response, are commensurate with a reasonable benefit/risk ratio, and/or are effective for their intended use.

The term “therapeutically effective” is intended to qualify the amount of active ingredients used in the treatment of a disease or disorder or on the effecting of a clinical endpoint.

The invention is further illustrated by the following examples.

### EXAMPLES

### Example 1—a Method of Making Genome-Edited CAR-T Cells

The following steps may be taken to provide the gene-edited CAR-T cells disclosed herein. As those of skill in the art will recognize, certain of the steps may be conducted sequentially or out of the order listed below, though perhaps leading to different efficiency.

Step 1: Isolation.

Peripheral blood mononuclear cells (PBMCs) are harvested from one or more healthy donors.

Step 2: Purification.

T cells are then isolated/purified from a donor's PBMCs (cord blood is an alternative source), for example using magnetic selection with a labelled antibody-coated magnetic beads (e.g., Miltenyi Biotech). Other purification techniques are known in the art and may be used.

Step 3: Genome Editing.

If the cell is expected to be used in an allogeneic adoptive cell transfer therapy, the TCR may be deleted from the cell surface or inactivated by editing a target genetic sequence of the TCR or a subunit thereof (e.g., TRAC). If a CAR targeting one or more antigens is to be transduced into the T cell, the antigen that is the target of the CAR may be deleted from the cell surface or its expression suppressed to prevent subsequent fratricide. In either case or both, deletion/suppression/inactivation may be accomplished by electroporating with Cas9 mRNA or protein, and gRNA against a portion of the gene sequences of the target(s). Cas9 mRNA/protein and gRNA against the target sequence can be electroporated together or in sequence, i.e., electroporate Cas9 mRNA/protein, then electroporate gRNA against the target(s). Additionally, gRNAs to different target sequences can be incorporated into a single vector for multiplex genome editing (i.e., simultaneous editing of multiple genes). Genome editing prior to activation is a potentially viable way to activate and genome-edit T cells with at least equal efficiency to editing activated cells.

It might also enhance transduction efficiency because viral vector carrying the CAR can be added earlier after activation, during the presence of stimulation. This is successful because there is a delay between genome-editing and the loss of protein, i.e., the TCR on the surface of the CAR-T, so the CAR can still be activated. Other techniques, however, could be used to suppress expression of the target. These include other genome editing techniques such as TALENs, ZFNs, RNA interference, and eliciting of internal binding of the antigen to prevent cell surface expression. Examples of gRNAs that may be used include those shown in table 9, and others known in the art.

Examples of guide RNA sequences are given below and are known to those of skill in the art.

TABLE 9
Guide RNA sequences

| Target Gene | Guide RNA Sequence (gRNA) |
|---|---|
| CS1 | 5′_2′OMe(G(ps)A(ps)C(ps))CAAUCUGACAUGCUGCAGUUUUAGAGCU |
| | AGAAAUAGCAAGUUAAAAUAAGGCUAGUCCGUUAUCAACUUGAA |
| | AAAGUGGCACCGAGUCGGUGC2′OMe(U(ps)U(ps)U(ps)U_3′ (SEQ ID |
| | NO: 9) |
| CD38 g3 | 5′_2′OMe(A(ps)A(ps)U(ps))UCAUCCUGAGAUGAGGUGUUUUAGAGCU |
| | AGAAAUAGCAAGUUAAAAUAAGGCUAGUCCGUUAUCAACUUGAA |
| | AAAGUGGCACCGAGUCGGUGC 2′OMe(U(ps)U(ps)U(ps)U_3′ (SEQ ID |
| | NO: 10) |
| CD38 g4 | 5′_2′OMe(C(ps)A(ps)U(ps))CCUGAGAUGAGGUGGGUGUUUUAGAGCU |
| | AGAAAUAGCAAGUUAAAAUAAGGCUAGUCCGUUAUCAACUUGAA |
| | AAAGUGGCACCGAGUCGGUGC 2′OMe(U(ps)U(ps)U(ps)U_3′ (SEQ ID |
| | NO: 11) |
| CD7 g10 | 5′_2′OMe(G(ps)U(ps)A(ps))GACAUUGACCUCCGUGAGUUUUAGAGCU |
| | AGAAAUAGCAAGUUAAAAUAAGGCUAGUCCGUUAUCAACUUGAA |
| | AAAGUGGCACCGAGUCGGUGC2′OMe(U(ps)U(ps)U(ps)U_3′ (SEQ ID |
| | NO: 12) |
| CD7 g4 | 5′_2′OMe(A(ps)U(ps)C(ps))ACGGAGGUCAAUGUCUAGUUUUAGAGCU |
| | AGAAAUAGCAAGUUAAAAUAAGGCUAGUCCGUUAUCAACUUGAA |
| | AAAGUGGCACCGAGUCGGUGC2′OMe(U(ps)U(ps)U(ps)U_3′ (SEQ ID |
| | NO: 13) |
| TRAC g | 5′_2′OMe(G(ps)A(ps)G(ps))AAUCAAAAUCGGUGAAUGUUUUAGAGCU |
| | AGAAAUAGCAAGUUAAAAUAAGGCUAGUCCGUUAUCAACUUGAA |
| | AAAGUGGCACCGAGUCGGUGC2′OMe(U(ps)U(ps)U(ps) U 3′ (SEQ ID |
| | NO: 14) |
| CD2 g | 5′_2′OMe(A(ps)C(ps)A(ps))GCUGACAGGCUCGACACGUUUUAGAGCU |
| | AGAAAUAGCAAGUUAAAAUAAGGCUAGUCCGUUAUCAACUUGAA |
| | AAAGUGGCACCGAGUCGGUGC2′OMe(U(ps)U(ps)U(ps) U 3′ (SEQ ID |
| | NO: 15) |
| CD3ε g | 5′_2′OMe(A(ps)G(ps)G(ps))GCAUGUCAAUAUUACUGGUUUUAGAGCU |
| | AGAAAUAGCAAGUUAAAAUAAGGCUAGUCCGUUAUCAACUUGAA |
| | AAAGUGGCACCGAGUCGGUGC2′OMe(U(ps)U(ps)U(ps)U 3′ (SEQ ID |
| | NO: 16) |
| CD5 g | 5′_2′OMe(C(ps)G(ps)U(ps))uCCAACUCGAAGUGCCAGUUUUAGAGCU |
| | AGAAAUAGCAAGUUAAAAUAAGGCUAGUCCGUUAUCAACUUGAA |
| | AAAGUGGCACCGAGUCGGUGC2′OMe(U(ps)U(ps)U(ps)U_3′ (SEQ ID |
| | NO: 17) |

\hat{ }RNA; (ps) indicate phosphorothioate.
\hat{ }\hat{ }Underlined bases denote target sequence.

Step 4: Activation.

T cells are thereafter activated. Human primary T cells were activated using anti-CD3 antibodies and anti-CD28 antibodies. Alternatively, anti-CD3 antibodies, anti-CD2 antibodies, and anti-CD28 antibodies may be used. Soluble antibodies may be used for activation, but antibody-coated beads are more often used, e.g. magnetic beads such as Dynabeads. In the case of the deletion of the TCR, the TCR is composed of proteins expressed prior to genome editing in sufficient quantities to allow for activation of the TCR until loss of these protein occur. Activating agents may be removed by applying a magnetic field, or, if an antibody matrix is used, by dilution with phosphate-buffered saline or other media, centrifuging, removing the supernatant, resuspending in fresh media, etc. (washing).

Step 5: CAR Transduction.

T cells may then be transduced with a CAR targeted to (i.e., that recognizes) one or more antigen or protein targets, for example with a lentivirus containing a CAR construct. Any other suitable method of transduction may be used, for example. The CAR may be electroporated into the cell using a variety of suitable equipment, e.g. electroporation devices from Miltenyi Biotec or Lonza.

Step 6: Expansion.

Remove CD3/CD28 stimulation and expand CAR-T population. This can continue for one week, two weeks or several weeks.

Additional Steps.

TCR⁺ cells may be depleted to produce a TCR⁻ cell population, e.g., by using beads coated in antibodies which bind to the TCR or a subunit thereof (e.g., Miltenyi Biotec alpha beta kit).

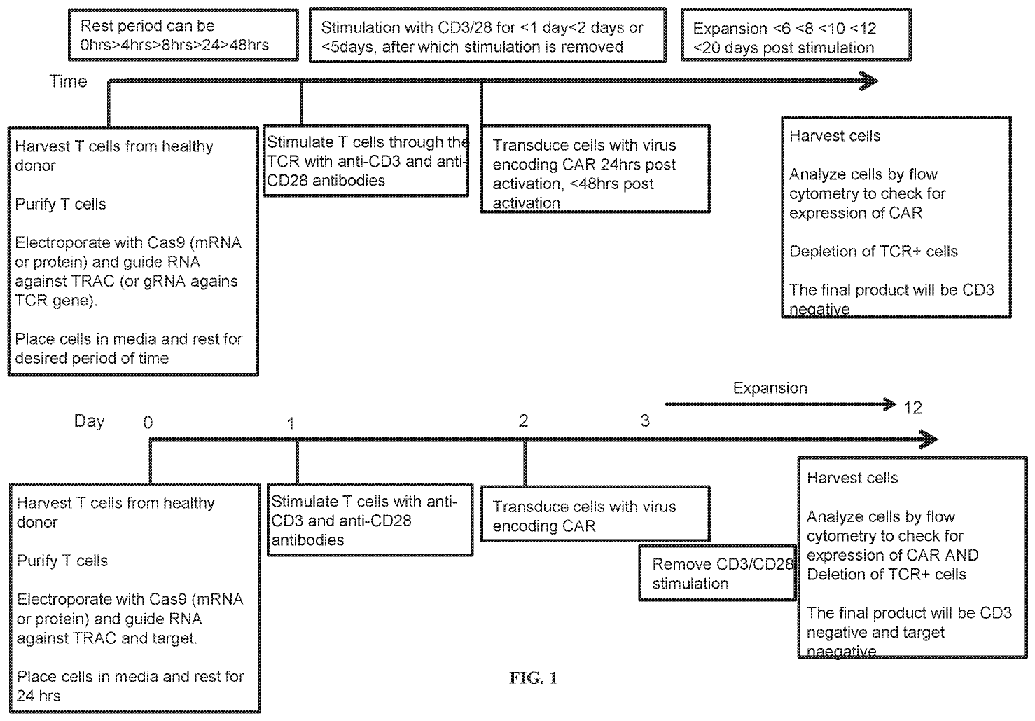

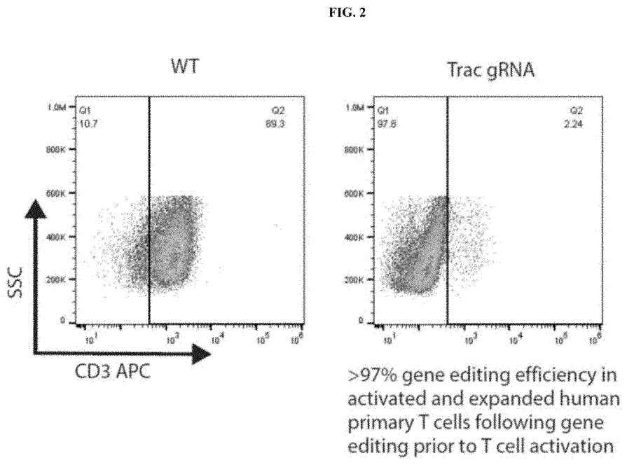

These steps are shown as a flow diagram in FIG. 1. Those of skill in the art will appreciate that some flexibility is possible in the conditions and time frames specified in FIG. 1. In FIG. 2, CAR-T cells are analyzed by flow cytometry to check for expression of CAR and deletion of TCR⁺ cells. In certain embodiments, the final product will be deficient in expression of the gene edited target(s). In certain embodiments, it will be CAR-bearing and deficient in functional TCR; in further embodiments, alternatively or in addition, it will be deficient in the cell surface protein(s)/antigen(s) that is/are the target(s) of the CAR. In this way, cells made by the method above will accordingly be fratricide-resistant and will not cause graft-vs.-host disease.

Variation: PEBL.

In an variation of the method above, a construct encoding one or more protein expression blocker (PEBL) may be transduced into the cell, either as the editing step or part of the editing step, or as part of CAR transduction. For example, an construct encoding an antibody-derived single-chain variable fragment specific for CD3ε may be transduced, e.g. by a lentiviral vector. Once expressed, the PEBL colocalizes intracellularly with CD3ε, blocking surface CD3 and TCRαβ expression. Accordingly, PEBL blockade of surface CD3/TCRαβ expression is an alternative method of preparing allogeneic CAR-T cells. Furthermore, PEBL and CAR expression can be combined in a single construct. Either of these methods may be achieved using the methods disclosed herein, and PEBLs may be produced for blockade of any of the targets of gene suppression disclosed herein.

Variation: PEBL.

In an variation of the method above, a construct encoding one or more shRNAs may be transduced into the cell, either as the editing step or part of the editing step, or as part of CAR transduction. Such shRNAs are also useful for the blockade of any of the targets of gene suppression disclosed herein.

Variation: Cytokines and Other Proteins.

In an variation of the method above, a construct encoding one or more cytokines or cytokine receptors may be transduced into the cell, either as the editing step or part of the editing step, or as part of CAR transduction. For example, an construct encoding such a cytokine or receptor, e.g., IL-7R or a mutant thereof, or IL-15 or a mutant thereof, may be transduced, e.g. by a lentiviral vector.

The foregoing methods are amenable to a variety of suitable conditions. Different growth media may be employed, and cells may be cultured at varying temperatures, e.g., between about room temperature (25° C.) and about 40° C., often between about 30° C. and about 37° C.

### Example 2—Genome-Edited UCART Cells Made by Editing Before Activation

On Day 0, cells were thawed in a thaw buffer. Thereafter, cells were resuspended in media and allowed to rest after editing for two hours. Cells were harvested and counted. The required number of cells were centrifuged at 100×g for 10 minutes at room temperature. Supernatant was removed completely, cells resuspended in PBS (1 ml) and transfer to a microcentrifuge tube, and centrifuged at 100×g for 10 minutes at room temperature. Supernatant was removed completely, and cells then resuspended in a pre-warmed buffer P3, counted, and the count adjusted to 5×10⁷ per mL. A cell pool volume of 100 μL was added to a tube containing Cas9/gRNA, gently mixed, and everything transferred into the Nucleocuvette™, which was gently tapped to remove bubbles. Electroporation was thereafter commenced using program (Human T cell stim EO-115). After this procedure, the activated cells were transferred to pre-warmed media and distributed in 2 mL aliquots in a 12-well plate. Aliquoted samples were rested for 24 hours.

On day 1, cells are activated with T Cell TransAct™ as shown in Table 10.

TABLE 10

| | Name | Media | Stimulation | Cas9 p | 2RNA | Virus |
|---|---|---|---|---|---|---|
| 1 | WT | TexMacs | T Cell | — | — | |
| | | | TransAct ™ | | | |
| | | | (50 μl) | | | |
| 2 | WC5 | TexMacs | T Cell | 2 ul | 20 ug iDT | WC5 |
| | | | TransAct ™ | | CD2 + CD3ε | |
| | | | (50 μl) | | | |
| 3 | WC6 | TexMacs | T Cell | 2 ul | 20 ug iDT | WC6 |
| | | | TransAct ™ | | CD2 + CD3ε | |
| | | | (50 μl) | | | |
| 4 | WC7 | TexMacs | T Cell | 2 ul | 20 ug iDT | WC7 |
| | | | TransAct ™ | | CD2 + CD3ε | |
| | | | (50 μl) | | | |
| 5 | WC8 | TexMacs | T Cell | 2 ul | 20 ug iDT | WC8 |
| | | | TransAct ™ | | CD2 + CD3ε | |
| | | | (50 μl) | | | |
| 6 | WC13 | TexMacs | T Cell | 2 ul | 20 ug iDT | WC13 |
| | | | TransAct ™ | | CD2 + CD3ε | |
| | | | (50 μl) | | | |
| 7 | WC14 | TexMacs | T Cell | 2 ul | 20 ug iDT | WC14 |
| | | | TransAct ™ | | CD2 + CD3ε | |
| | | | (50 μl) | | | |
| 8 | WC15 | TexMacs | T Cell | 2 ul | 20 ug iDT | WC15 |
| | | | TransAct ™ | | CD2 + CD3ε | |
| | | | (50 μl) | | | |
| 9 | WC16 | TexMacs | T Cell | 2 ul | 20 ug iDT | WC16 |
| | | | TransAct ™ | | CD2 + CD3ε | |
| | | | (50 μl) | | | |

On day 2, 1 μl of polybrene was added for each ml media (8 mg/ml stock). The required amount of virus was added to give required M.O.I (multiplicity of infection). Cells and virus were mixed and placed back in incubator at 37° C.

On day 3, activated cells were washed to remove stimulation.

On Day 12, FACS analysis showed the high purity of CD3-CD2-/CAR-T cells. Standard four-hour chromium release (⁵¹Cr) assays were performed using (51Cr) labeled genome-edited Jurkat cells (ΔCD2, ΔCD3 and ΔCD2ΔCD3. These experiments showed a functional tumor killing response to CD2 and CD3 targets independent of one another.

The foregoing methods were used to generate a variety of universal (TCR-deleted) CAR-T cells, e.g., UCART cells targeting CD7 (UCART7), tUCART2/3, and CD3 (UCART3).

### Example 3: Kinetics of Genome-Editing, Activation, and Expansion in T Cells

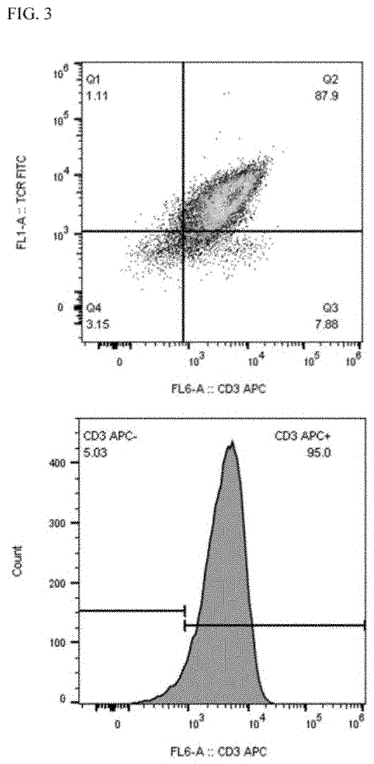

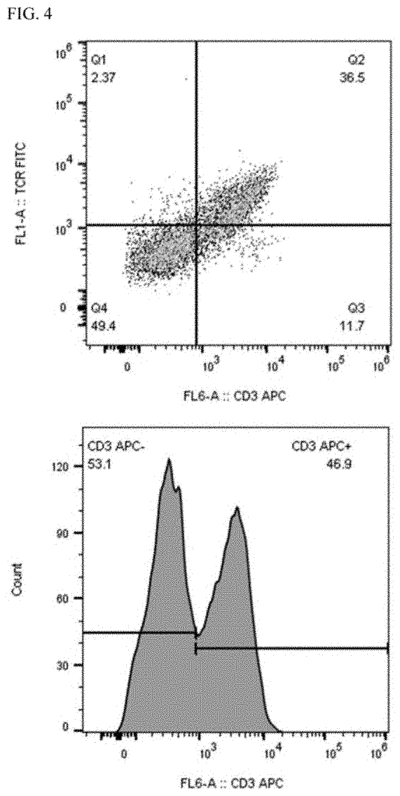

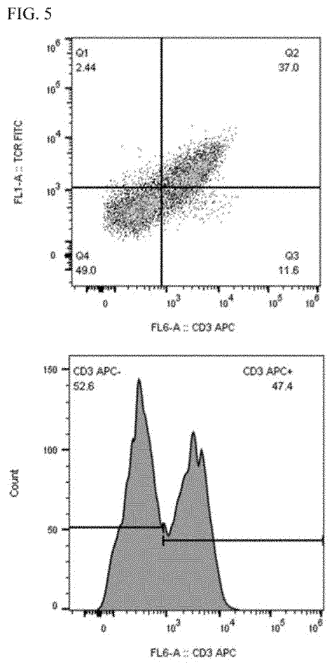

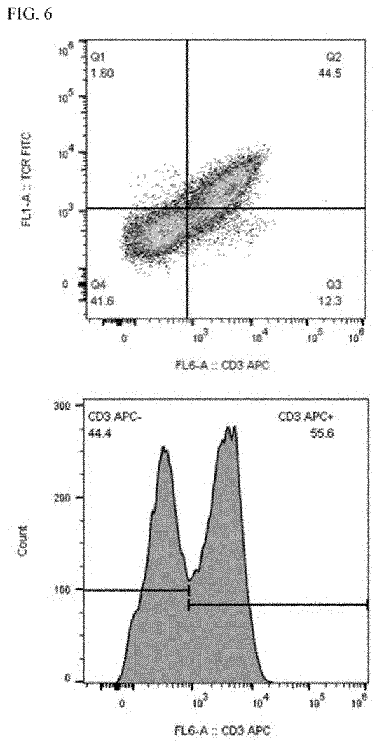

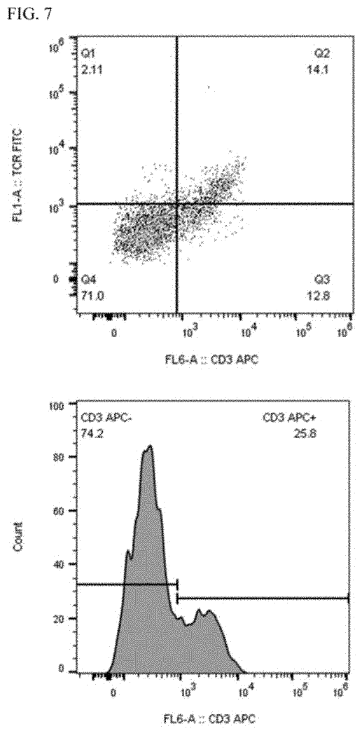

As shown in FIG. 3, naive T cells were activated with TransAct reagent (Miltenyi) according to manufacturer's instructions in TexMacs media (Miltenyi) containing 10 ng/mL IL-15 and 10 ng/mL IL-7, at 37° C. As shown in FIGS. 4-7, naive T cells were electroporated using the nucleofector 4D (Lonza program EO-115) with 20 ug TRAC gRNA and 15 ug Cas9 mRNA in 100 ul Lonza buffer P3. After electroporation, cells were rested for 0 hrs (FIG. 4), 4 hours (FIG. 5), 8 hours (FIG. 6), or 20 hours (FIG. 7) in TexMacs media (Miltenyi) containing 10 ng/mL IL-15 and 10 ng/mL IL-7, at 37° C. and then activated with TransAct reagent (Miltenyi) according to manufacturer's instructions.

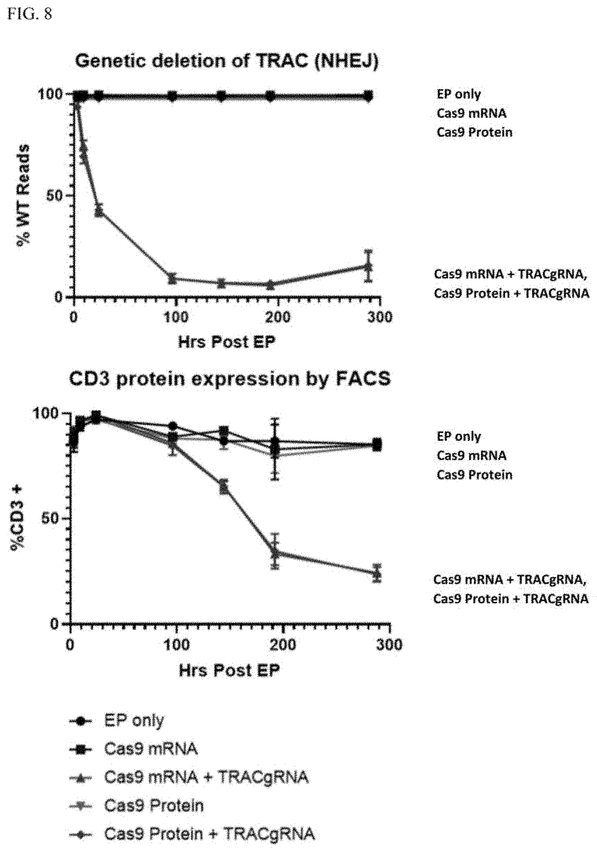

T cells were electroporated using the Nucleofector 4D (Lonza program EO-115) with 20 ug TRAC gRNA and Cas9 (15 ug Cas9 mRNA or 10 ug Cas9 protein) in 100 ul Lonza buffer P3. After electroporation, cells were rested for 20 hrs in TexMacs media (Miltenyi) containing 10 ng/mL IL-15 and 10 ng/mL IL-7, at 37° C. and then activated with TransAct reagent (Miltenyi) according to manufacturer's instructions. Stimulation was removed by washing the cells after incubation for 48 hrs As shown in the upper panel of FIG. 8, DNA was extracted from T cells at multiple time points post editing, and gene editing efficiency assed using targeted deep sequencing of the TRAC locus. As shown in the lower panel of FIG. 8, TCR expression was analyzed at multiple time points post editing, using FACS. CD3ε surface protein expression was used a surrogate market for TCR expression. TCR surface expression lags genetic deletion. This provides a window of activation allowing activation of the T cells through TCR signalling.

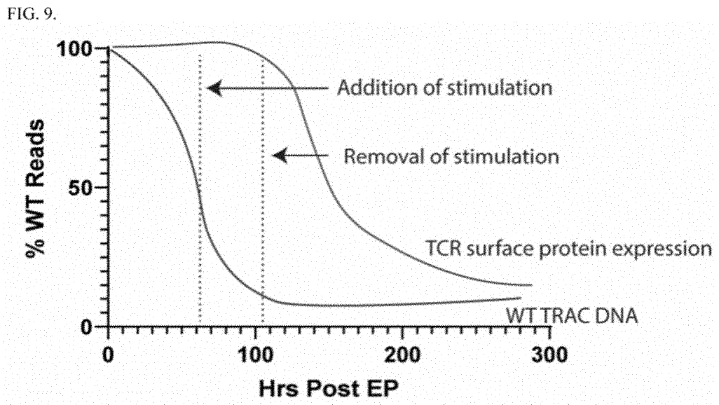

FIG. 9 shows a theoretical T cell activation window. As the experiments above demonstrate, TCR surface expression lags genetic deletion and provides a window in which cells can be activated through TCR surface protein expression after loss of TRAC gene function. Activation of the T cells through the TCR after gene editing has occurred will reduce p53 mediated cell cycle arrest induced by the formation of double strand breaks in actively dividing cells and enhance expansion. Removal of T cell stimulation just prior to loss of TCR surface protein will maximize expansion and stimulation of gene edited cells and minimize preferential expansion of TCR+ T cells that escaped gene editing.

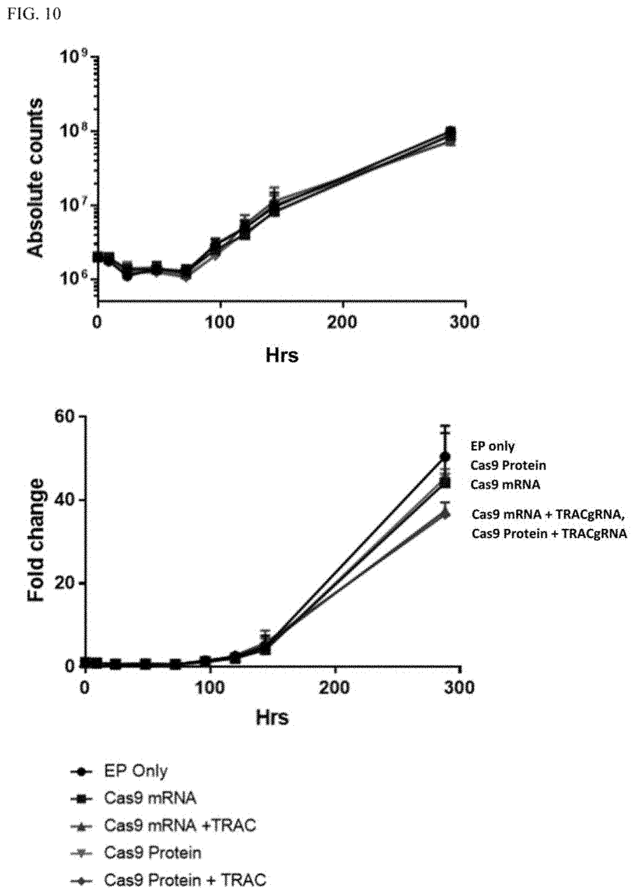

FIG. 10 shows the kinetics of T cell expansion. T cells were electroporated using the nucleofector 4D (Lonza program EO-115) with 20 μg TRAC gRNA and Cas9 (15 μg Cas9 mRNA or 10 μg Cas9 protein) in 100 ul Lonza buffer P3. After electroporation, cells were rested for 20 hrs in TexMacs media (Miltenyi) containing 10 ng/mL IL-15 and 10 ng/mL IL-7, at 37° C. and then activated with TransAct reagent (Miltenyi) according to manufacturer's instructions. Stimulation was removed by washing the cells after incubation for 48 hours. The upper panel shows absolute cell counts, the lower panel fold expansion. Robust expansion was observed across groups.

The methods disclosed above and herein may be varied; for example, sequential genome editing steps may be employed. For example, multiple rounds of genome editing (electroporation) may be performed before activation; or, alternatively, one or more subsequent rounds of editing may be performed after a first editing and activation.

### Example 4: Gene Editing by CAR Insertion into Gene Locus

A CAR or any protein of interest may be inserted into a gene locus, for example the gene for the T cell receptor. MacLeod et al. (“Integration of a CD19 CAR into the TCR Alpha Chain Locus Streamlines Production of Allogeneic Gene-Edited CAR T Cells,” Molec Therapy 25(4):P949-961, 2017) reports the generation of allogeneic CAR T cells by targeting the insertion of a CAR transgene directly into the native TCR locus using an engineered homing endonuclease and an AAV donor template. Anti-CD19 CAR T cells produced in this manner do not express the endogenous cell-surface TCR, exhibit potent effector functions in vitro, and mediate clearance of CD19+ tumors in an in vivo mouse model. The resulting gene-edited CAR T cells exhibit potent anti-tumor activity in vitro and in vivo in preclinical models, suggesting that these cells have potential for safe and efficacious use as adoptive cellular therapy in unrelated patients with CD19+ hematological malignancies.

The methods described above may be adapted to insert a CAR into a locus for a gene encoding an antigen, cell surface protein, or secretable protein, such as a cytokine. In this way, editing of the genome is effected by transfection of CAR. Thereafter, cells may be activated as described herein, removing separate genome editing step in certain embodiments. Ideally, such a step should be performed while cells are actively dividing. Such methods are also expected to result in robust expansion of engineered cells.

### Example 5: Guide RNA Selection

Guide RNA were designed and validated for activity by Washington University Genome Engineering & iPSC. Sequences complementary to a given gRNA may exist throughout the genome, including but not limited to the target locus. A short sequence is likelier to hybridize off-target. some long sequences within the gRNA may have exact matches (long_0) Of near matches (long_1, long_2, representing, respectively, a single or two nucleotide difference) throughout the genome. These may also hybridize off-target, in effect leading to editing of the wrong gene and diminishing editing efficiency.

hCD2.

Off target analysis of selected gRNA was performed for 2 exons of hCD2 (CF58 and CF59) to determine the number of sites in human genome which are an exact match or contains up to 1 or 2 mismatches, which may include the target site. The results are listed in Table 11 for Exon CF58 and Table 12 for Exon CF59.

TABLE 11
Guide RNA (gRNA) Off Target Analysis for hCD2 (Exon CF58)

| Name | gRNA | long_0 | long_1 | long_2 | short_0 | SNP |
|---|---|---|---|---|---|---|
| CF58.CD2.g1 | CAAAGAGATTACGAATGCCTN | 1 | 1 | 1 | 3 | NA |
| | GG (SEQ ID NO: 364) | | | | | |
| CF58.CD2.g23 | CAAGGCATTCGTAATCTCTTNG | 1 | 1 | 1 | 5 | NA |
| | G (SEQ ID NO: 365) | | | | | |
| CF58.CD2.g18 | CTTGTAGATATCCTGATCATNG | 1 | 1 | 1 | 13 | NA |
| | G (SEQ ID NO: 366) | | | | | |
| CF58.CD2.g8 | CTTGGGTCAGGACATCAACTN | 1 | 1 | 1 | 14 | NA |
| | GG (SEQ ID NO: 367) | | | | | |
| CF58.CD2.g14 | CGATGATCAGGATATCTACAN | 1 | 1 | 1 | 17 | NA |
| | GG (SEQ ID NO: 368) | | | | | |
| CF58.CD2.g2 | TTACGAATGCCTTGGAAACCN | 1 | 1 | 1 | 27 | NA |
| | GG (SEQ ID NO: 369) | | | | | |
| CF58.CD2.g3 | TACGAATGCCTTGGAAACCTN | 1 | 1 | 1 | 34 | NA |
| | GG (SEQ ID NO: 370) | | | | | |
| CF58.CD2.g4 | ACGAATGCCTTGGAAACCTGN | 1 | 1 | 1 | 40 | NA |
| | GG (SEQ ID NO: 371) | | | | | |
| CF58.CD2.g10 | TGATATTGACGATATAAAATN | 1 | 1 | 2 | 3 | NA |
| | GG (SEQ ID NO: 372) | | | | | |
| CF58.CD2.g9 | ATGATATTGACGATATAAAAN | 1 | 1 | 2 | 4 | NA |
| | GG (SEQ ID NO: 373) | | | | | |
| CF58.CD2.g13 | GCATCTGAAGACCGATGATCN | 1 | 1 | 2 | 4 | NA |
| | GG (SEQ ID NO: 374) | | | | | |
| CF58.CD2.g7 | AACCTGGGGTGCCTTGGGTCN | 1 | 1 | 2 | 22 | NA |
| | GG (SEQ ID NO: 375) | | | | | |
| CF58.CD2.g6 | TTGGAAACCTGGGGTGCCTTNG | 1 | 1 | 2 | 33 | NA |
| | G (SEQ ID NO: 376) | | | | | |
| CF58.CD2.g15 | GTATCAATATATGATACAAAN | 1 | 1 | 2 | 35 | NA |
| | GG (SEQ ID NO: 377) | | | | | |
| CF58.CD2.g25 | CAAGGCACCCCAGGTTTCCAN | 1 | 1 | 2 | 45 | NA |
| | GG (SEQ ID NO: 378) | | | | | |
| CF58.CD2.g5 | CTTGGAAACCTGGGGTGCCTN | 1 | 1 | 2 | 62 | NA |
| | GG (SEQ ID NO: 379) | | | | | |
| CF58.CD2.g19 | TCATCACTCATTTGAAAACTNG | 1 | 1 | 3 | 56 | NA |
| | G (SEQ ID NO: 380) | | | | | |
| CF58.CD2.g20 | CAAGTTGATGTCCTGACCCANG | 1 | 1 | 4 | 27 | NA |
| | G (SEQ ID NO: 381) | | | | | |
| CF58.CD2.g21 | GTCCTGACCCAAGGCACCCCN | 1 | 1 | 4 | 33 | NA |
| | GG (SEQ ID NO: 382) | | | | | |
| CF58.CD2.g17 | ATATTTGATTTGAAGATTCANG | 1 | 1 | 6 | 35 | NA |
| | G (SEQ ID NO: 383) | | | | | |
| CF58.CD2.g16 | TACAAAAGGAAAAAATGTGTN | 1 | 1 | 7 | 64 | NA |
| | GG (SEQ ID NO: 384) | | | | | |
| CF58.CD2.g12 | ACATATAAGCTATTTAAAAAN | 1 | 1 | 8 | 58 | NA |
| | GG (SEQ ID NO: 385) | | | | | |
| CF58.CD2.g11 | AAAAGAGAAAGAGACTTTCAN | 1 | 1 | 15 | 42 | NA |
| | GG (SEQ ID NO: 386) | | | | | |

TABLE 12
Guide RNA (gRNA) Off Target Analysis for hCD2 (CF59)

| Name | gRNA | long_0 | long_1 | long_2 | short_0 | SNP |
|---|---|---|---|---|---|---|
| CF59.CD2.g20 | CTTGATACAGGTTTAATTCGNG | 1 | 1 | 1 | 2 | NA |
| | G (SEQ ID NO: 387) | | | | | |
| CF59.CD2.g13 | ACAGCTGACAGGCTCGACACN | 1 | 1 | 1 | 4 | NA |
| | GG (SEQ ID NO: 388) | | | | | |
| CF59.CD2.g17 | GATGTTTCCCATCTTGATACNG | 1 | 1 | 1 | 8 | NA |
| | G (SEQ ID NO: 389) | | | | | |
| CF59.CD2.g12 | GTCGAGCCTGTCAGCTGTCCNG | 1 | 1 | 1 | 24 | NA |
| | G (SEQ ID NO: 390) | | | | | |
| CF59.CD2.g10 | CAAAATTCAAGTGCACAGCAN | 1 | 1 | 1 | 33 | NA |
| | GG (SEQ ID NO: 391) | | | | | |
| CF59.CD2.g16 | GAATTTTGCACTCAGGCTGGNG | 1 | 1 | 1 | 245 | NA |
| | G (SEQ ID NO: 392) | | | | | |
| CF59.CD2.g4 | GAATTAAACCTGTATCAAGAN | 1 | 1 | 2 | 7 | NA |
| | GG (SEQ ID NO: 393) | | | | | |
| CF59.CD2.g5 | AATTAAACCTGTATCAAGATNG | 1 | 1 | 2 | 7 | NA |
| | G (SEQ ID NO: 394) | | | | | |
| CF59.CD2.g21 | AGTTCCATTCATTACCTCACNG | 1 | 1 | 2 | 14 | NA |
| | G (SEQ ID NO: 395) | | | | | |
| CF59.CD2.g8 | AGAGGGTCATCACACACAAGN | 1 | 1 | 2 | 20 | NA |
| | GG (SEQ ID NO: 396) | | | | | |
| CF59.CD2.g25 | ATACAAGTCCAGGAGATCTTNG | 1 | 1 | 2 | 21 | NA |
| | G (SEQ ID NO: 397) | | | | | |
| CF59.CD2.g19 | TCTTGATACAGGTTTAATTCNG | 1 | 1 | 2 | 25 | NA |
| | G (SEQ ID NO: 398) | | | | | |
| CF59.CD2.g3 | CTGACCTGTGAGGTAATGAAN | 1 | 1 | 2 | 29 | NA |
| | GG (SEQ ID NO: 399) | | | | | |
| CF59.CD2.g7 | ACATCTAAAACTTTCTCAGANG | 1 | 1 | 2 | 41 | NA |
| | G (SEQ ID NO: 400) | | | | | |
| CF59.CD2.g9 | GCAAAATTCAAGTGCACAGCN | 1 | 1 | 2 | 46 | NA |
| | GG (SEQ ID NO: 401) | | | | | |
| CF59.CD2.g24 | GGTTGTGTTGATACAAGTCCNG | 1 | 1 | 3 | 8 | NA |
| | G (SEQ ID NO: 402) | | | | | |
| CF59.CD2.g18 | ATCTTGATACAGGTTTAATTNG | 1 | 1 | 3 | 24 | NA |
| | G (SEQ ID NO: 403) | | | | | |
| CF59.CD2.g23 | ATTCATTACCTCACAGGTCANG | 1 | 1 | 3 | 35 | NA |
| | G (SEQ ID NO: 404) | | | | | |
| CF59.CD2.g6 | AACATCTAAAACTTTCTCAGNG | 1 | 1 | 3 | 43 | NA |
| | G (SEQ ID NO: 405) | | | | | |
| CF59.CD2.g11 | AGCAGGGAACAAAGTCAGCAN | 1 | 1 | 3 | 45 | NA |
| | GG (SEQ ID NO: 406) | | | | | |
| CF59.CD2.g2 | CAACACAACCCTGACCTGTGNG | 1 | 1 | 3 | 47 | NA |
| | G (SEQ ID NO: 407) | | | | | |
| CF59.CD2.g15 | CTTGAATTTTGCACTCAGGCNG | 1 | 1 | 4 | 21 | NA |
| | G (SEQ ID NO: 408) | | | | | |
| CF59.CD2.g22 | CATTCATTACCTCACAGGTCNG | 1 | 1 | 10 | 29 | NA |
| | G (SEQ ID NO: 409) | | | | | |
| CF59.CD2.g14 | TGCACTTGAATTTTGCACTCNG | 1 | 2 | 3 | 26 | NA |
| | G (SEQ ID NO: 410) | | | | | |
| CF59.CD2.g1 | TCTCAAAACCAAAGATCTCCNG | 1 | 2 | 5 | 19 | NA |
| | G (SEQ ID NO: 411) | | | | | |

The gRNA sequences in Table 11 and Table 12 were normalized (% Normalization to NHEJ) for gRNA activity via next generation sequencing (NGS). GFP was used as a control. Following sequencing analysis, the following gRNAs were recommended based on off-target profile: CF58.CD2.g1 (41.2%), CF58.CD2.g23 (13.2%), CF59.CD2.g20 (26.6%), CF59.CD2.g13 (66.2%), CF59.CD2.g17 (17.5%). Guide RNA (gRNA) with normalized NHEJ frequencies equal to or greater than 15% are good candidates for cell line and animal model creation projects.

hCD3E.

Off target analysis of selected gRNA was performed for hCD3E to determine the number of sites in human genome which are an exact match or contains up to 1 or 2 mismatches, which may include the target site. The results are listed in Table 13 for hCD3E.

TABLE 13
Guide RNA (gRNA) Off Target Analysis for hCD3E

| Name | gRNA | long_0 | long_1 | long_2 | long_3 | short_0 | SNP |
|---|---|---|---|---|---|---|---|
| MS1044.CD3E.sp2 | TTGACATGCCCTCAGTATC | 1 | 1 | 1 | 21 | 73 | NA |
| | CNGG (SEQ ID NO: 22) | | | | | | |
| MS1044.CD3E.sp17 | CTGGATTACCTCTTGCCCT | 1 | 1 | 1 | 24 | 114 | NA |
| | CNGG (SEQ ID NO: 23) | | | | | | |
| MS1044.CD3E.sp28 | GAGATGGAGACTTTATAT | 1 | 1 | 1 | 30 | 44 | NA |
| | GCNGG (SEQ ID NO: 24) | | | | | | |
| MS1044.CD3E.sp29 | AGATGGAGACTTTATATG | 1 | 1 | 1 | 33 | 55 | NA |
| | CTNGG (SEQ ID NO: 25) | | | | | | |
| MS1044.CD3E.sp26 | AGGGCATGTCAATATTAC | 1 | 1 | 1 | 23 | 60 | NA |
| | TGNGG (SEQ ID NO: 26) | | | | | | |
| MS1044.CD3E.sp30 | GATGGAGACTTTATATGCT | 1 | 1 | 2 | 26 | 64 | NA |
| | GNGG (SEQ ID NO: 27) | | | | | | |
| MS1044.CD3E.sp12 | TATTATGTCTGCTACCCCA | 1 | 1 | 2 | 20 | 61 | NA |
| | GNGG (SEQ ID NO: 28) | | | | | | |
| MS1044.CD3E.sp23 | TGCCATAGTATTTCAGATC | 1 | 1 | 2 | 21 | 55 | NA |
| | CNGG (SEQ ID NO: 29) | | | | | | |
| MS1044.CD3E.sp18 | AGATAAAAGTTCGCATCT | 1 | 1 | 2 | 33 | 6 | NA |
| | TCNGG (SEQ ID NO: 30) | | | | | | |
| MS1044.CD3E.sp22 | CTGAAAATTCCTTCAGTGA | 1 | 1 | 2 | 44 | 60 | NA |
| | CNGG (SEQ ID NO: 31) | | | | | | |
| MS1044.CD3E.sp16 | CTGAGGGCAAGAGGTAAT | 1 | 1 | 3 | 30 | 41 | NA |
| | CCNGG (SEQ ID NO: 32) | | | | | | |
| MS1044.CD3E.sp25 | TTTCAGATCCAGGATACTG | 1 | 1 | 3 | 38 | 63 | NA |
| | ANGG (SEQ ID NO: 33) | | | | | | |
| MS1044.CD3E.sp15 | TATCTCTACCTGAGGGCA | 1 | 1 | 3 | 22 | 134 | NA |
| | AGNGG (SEQ ID NO: 34) | | | | | | |
| MS1044.CD3E.sp9 | TGAGGATCACCTGTCACT | 1 | 1 | 3 | 44 | 54 | NA |
| | GANGG (SEQ ID NO: 35) | | | | | | |

The gRNA sequences in Table 13 were normalized (% Normalization to NHEJ) for gRNA activity via next generation sequencing (NGS). GFP was used as a control. Following sequencing analysis, the following gRNAs were recommended based on off-target profile: MS1044.CD3E.sp28 (>15%) and MS1044.CD3E.sp12 (>15%). Guide RNA (gRNA) with normalized NHEJ frequencies equal to or greater than 15% are good candidates for cell line and animal model creation projects.

hCD5.

Off target analysis of selected gRNA was performed for 3 exons of hCD5 (Exon 3, Exon 4, and Exon 5) to determine the number of sites in human genome which are an exact match or contains up to 1 or 2 mismatches, which may include the target site. The results are listed in Table 14 for Exon 3, Table 15 for Exon 4, and Table 16 for Exon 5.

TABLE 14
Guide RNA (gRNA) Off Target Analysis for hCD5 (Exon 3)

| Name | gRNA | long_0 | long_1 | long_2 | short_0 | SNP |
|---|---|---|---|---|---|---|
| SP597.CD5.g22 | AATCATCTGCTACGGACAACN | 1 | 1 | 1 | 1 | NA |
| | GG (SEQ ID NO: 36) | | | | | |
| SP597.CD5.g39 | GCAGACTTTTGACGCTTGACN | 1 | 1 | 1 | 1 | NA |
| | GG (SEQ ID NO: 37) | | | | | |
| SP597.CD5.g1 | CCGTTCCAACTCGAAGTGCCN | 1 | 1 | 1 | 2 | NA |
| | GG (SEQ ID NO: 38) | | | | | |
| SP597.CD5.g2 | CGTTCCAACTCGAAGTGCCAN | 1 | 1 | 1 | 2 | NA |
| | GG (SEQ ID NO: 39) | | | | | |
| SP597.CD5.g50 | CTGGCACTTCGAGTTGGAACN | 1 | 1 | 1 | 2 | NA |
| | GG (SEQ ID NO: 40) | | | | | |
| SP597.CD5.g17 | GTCTGCCAGCGGCTGAACTGN | 1 | 1 | 1 | 3 | NA |
| | GG (SEQ ID NO: 41) | | | | | |
| SP597.CD5.g23 | ATCATCTGCTACGGACAACTN | 1 | 1 | 1 | 3 | NA |
| | GG (SEQ ID NO: 42) | | | | | |
| SP597.CD5.g41 | AGACTTTTGACGCTTGACTGNG | 1 | 1 | 1 | 3 | NA |
| | G (SEQ ID NO: 43) | | | | | |
| SP597.CD5.g40 | CAGACTTTTGACGCTTGACTNG | 1 | 1 | 1 | 5 | NA |
| | G (SEQ ID NO: 44) | | | | | |
| SP597.CD5.g49 | CCTGGCACTTCGAGTTGGAAN | 1 | 1 | 1 | 5 | NA |
| | GG (SEQ ID NO: 45) | | | | | |
| SP597.CD5.g38 | GCACCCCACAGTTCAGCCGCN | 1 | 1 | 1 | 8 | NA |
| | GG (SEQ ID NO: 46) | | | | | |
| SP597.CD5.g46 | CCTTGAGGTAGACCTCCAGCN | 1 | 1 | 1 | 9 | NA |
| | GG (SEQ ID NO: 47) | | | | | |
| SP597.CD5.g7 | AGGTCTACCTCAAGGACGGAN | 1 | 1 | 1 | 11 | NA |
| | GG (SEQ ID NO: 48) | | | | | |
| SP597.CD5.g51 | TGGAACGGGTGAGCCTTGCCN | 1 | 1 | 1 | 13 | NA |
| | GG (SEQ ID NO: 49) | | | | | |
| SP597.CD5.g20 | TGTGGGGTGCCCTTAAGCCTN | 1 | 1 | 1 | 19 | NA |
| | GG (SEQ ID NO: 50) | | | | | |
| SP597.CD5.g16 | AAGCGTCAAAAGTCTGCCAGN | 1 | 1 | 1 | 20 | NA |
| | GG (SEQ ID NO: 51) | | | | | |
| SP597.CD5.g29 | TAGCAGATGATTGAGCTCTGN | 1 | 1 | 1 | 25 | NA |
| | GG (SEQ ID NO: 52) | | | | | |
| SP597.CD5.g30 | GATTGAGCTCTGAGGTGTGTN | 1 | 1 | 1 | 33 | NA |
| | GG (SEQ ID NO: 53) | | | | | |
| SP597.CD5.g13 | GGGGCCGGAGCTCCAAGCAGN | 1 | 1 | 1 | 42 | NA |
| | GG (SEQ ID NO: 54) | | | | | |
| SP597.CD5.g33 | GGTGTGTAGGTGACAAGGAAN | 1 | 1 | 1 | 48 | NA |
| | GG (SEQ ID NO: 55) | | | | | |
| SP597.CD5.g15 | CCGGAGCTCCAAGCAGTGGGN | 1 | 1 | 1 | 58 | NA |
| | GG (SEQ ID NO: 56) | | | | | |
| SP597.CD5.g47 | GGTAGACCTCCAGCTGGCCCN | 1 | 1 | 1 | 78 | NA |
| | GG (SEQ ID NO: 57) | | | | | |
| SP597.CD5.g3 | CTCGAAGTGCCAGGGCCAGCN | 1 | 1 | 1 | 121 | NA |
| | GG (SEQ ID NO: 58) | | | | | |
| SP597.CD5.g48 | CTGGCCCTGGCACTTCGAGTN | 1 | 1 | 2 | 1 | NA |
| | GG (SEQ ID NO: 59) | | | | | |
| SP597.CD5.g18 | TCTGCCAGCGGCTGAACTGTN | 1 | 1 | 2 | 5 | NA |
| | GG (SEQ ID NO: 60) | | | | | |
| SP597.CD5.g45 | CCATGTGCCATCCGTCCTTGNG | 1 | 1 | 2 | 5 | NA |
| | G (SEQ ID NO: 61) | | | | | |
| SP597.CD5.g5 | CCAGCTGGAGGTCTACCTCAN | 1 | 1 | 2 | 14 | NA |
| | GG (SEQ ID NO: 62) | | | | | |
| SP597.CD5.g31 | TCTGAGGTGTGTAGGTGACAN | 1 | 1 | 2 | 18 | NA |
| | GG (SEQ ID NO: 63) | | | | | |
| SP597.CD5.g37 | AGGAAGGGGCCAAGGCTTAAN | 1 | 1 | 2 | 18 | NA |
| | GG (SEQ ID NO: 64) | | | | | |
| SP597.CD5.g21 | CAGAGCTCAATCATCTGCTAN | 1 | 1 | 2 | 19 | NA |
| | GG (SEQ ID NO: 65) | | | | | |
| SP597.CD5.g14 | GGGCCGGAGCTCCAAGCAGTN | 1 | 1 | 2 | 23 | NA |
| | GG (SEQ ID NO: 66) | | | | | |
| SP597.CD5.g43 | CCTCCCACTGCTTGGAGCTCNG | 1 | 1 | 2 | 30 | NA |
| | G (SEQ ID NO: 67) | | | | | |
| SP597.CD5.g44 | TGGAGCTCCGGCCCCAGCTCN | 1 | 1 | 2 | 38 | NA |
| | GG (SEQ ID NO: 68) | | | | | |
| SP597.CD5.g34 | GTGTGTAGGTGACAAGGAAGN | 1 | 1 | 2 | 48 | NA |
| | GG (SEQ ID NO: 69) | | | | | |
| SP597.CD5.g11 | ATGGTTTGCAGCCAGAGCTGN | 1 | 1 | 2 | 108 | NA |
| | GG (SEQ ID NO: 70) | | | | | |
| SP597.CD5.g6 | CTGGAGGTCTACCTCAAGGAN | 1 | 1 | 3 | 16 | NA |
| | GG (SEQ ID NO: 71) | | | | | |
| SP597.CD5.g19 | CTGCCAGCGGCTGAACTGTGN | 1 | 1 | 3 | 25 | NA |
| | GG (SEQ ID NO: 72) | | | | | |
| SP597.CD5.g25 | AATGACATGTGTCACTCTCTNG | 1 | 1 | 3 | 25 | NA |
| | G (SEQ ID NO: 73) | | | | | |
| SP597.CD5.g9 | ACATGGTTTGCAGCCAGAGCN | 1 | 1 | 3 | 30 | NA |
| | GG (SEQ ID NO: 74) | | | | | |
| SP597.CD5.g10 | CATGGTTTGCAGCCAGAGCTN | 1 | 1 | 3 | 52 | NA |
| | GG (SEQ ID NO: 75) | | | | | |
| SP597.CD5.g26 | GACACATGTCATTTCTGCTGNG | 1 | 1 | 3 | 53 | NA |
| | G (SEQ ID NO: 76) | | | | | |
| SP597.CD5.g42 | ACTGGGGTCCTCCCACTGCTNG | 1 | 1 | 3 | 91 | NA |
| | G (SEQ ID NO: 77) | | | | | |
| SP597.CD5.g8 | CCTCAAGGACGGATGGCACAN | 1 | 1 | 4 | 5 | NA |
| | GG (SEQ ID NO: 78) | | | | | |
| SP597.CD5.g32 | AGGTGTGTAGGTGACAAGGAN | 1 | 1 | 4 | 49 | NA |
| | GG (SEQ ID NO: 79) | | | | | |
| SP597.CD5.g36 | AAGGAAGGGGCCAAGGCTTAN | 1 | 1 | 5 | 16 | NA |
| | GG (SEQ ID NO: 80) | | | | | |
| SP597.CD5.g4 | GAAGTGCCAGGGCCAGCTGGN | 1 | 1 | 5 | 93 | NA |
| | GG (SEQ ID NO: 81) | | | | | |
| SP597.CD5.g12 | TTTGCAGCCAGAGCTGGGGCN | 1 | 1 | 8 | 257 | NA |
| | GG (SEQ ID NO: 82) | | | | | |
| SP597.CD5.g24 | AAATGACATGTGTCACTCTCN | 1 | 1 | 10 | 33 | NA |
| | GG (SEQ ID NO: 83) | | | | | |
| SP597.CD5.g35 | AGGTGACAAGGAAGGGGCCAN | 1 | 1 | 10 | 202 | NA |
| | GG (SEQ ID NO: 84) | | | | | |
| SP597.CD5.g27 | ATTTCTGCTGTGGCTGCAGTNG | 1 | 2 | 4 | 70 | NA |
| | G (SEQ ID NO: 85) | | | | | |
| SP597.CD5.g28 | GCTGTGGCTGCAGTTGGAGAN | 1 | 2 | 19 | 49 | NA |
| | GG (SEQ ID NO: 86) | | | | | |

TABLE 15
Guide RNA (gRNA) Off Target Analysis for hCD5 (Exon 4)

| Name | gRNA | long_0 | long_1 | long_2 | short_0 | SNP |
|---|---|---|---|---|---|---|
| SP598.CD5.g10 | GGCGGGGGCCTTGTCGTTGG | 1 | 1 | 1 | 1 | NA |
| | NGG (SEQ ID NO: 87) | | | | | |
| SP598.CD5.g7 | CTCTGGAGTTGTGGTGGGCG | 1 | 1 | 1 | 16 | NA |
| | NGG (SEQ ID NO: 88) | | | | | |
| SP598.CD5.g8 | TCTGGAGTTGTGGTGGGCGG | 1 | 1 | 1 | 40 | NA |
| | NGG (SEQ ID NO: 89) | | | | | |
| SP598.CD5.g12 | CGTTGGAGGTGTTGTCTTCTN | 1 | 1 | 1 | 46 | NA |
| | GG (SEQ ID NO: 90) | | | | | |
| SP598.CD5.g1 | AGACAACACCTCCAACGACA | 1 | 1 | 2 | 2 | NA |
| | NGG (SEQ ID NO: 91) | | | | | |
| SP598.CD5.g9 | GTGGGCGGGGGCCTTGTCGT | 1 | 1 | 2 | 5 | NA |
| | NGG (SEQ ID NO: 92) | | | | | |
| SP598.CD5.g11 | TCGTTGGAGGTGTTGTCTTCN | 1 | 1 | 2 | 13 | NA |
| | GG (SEQ ID NO: 93) | | | | | |
| SP598.CD5.g2 | ACCACAACTCCAGAGCCCAC | 1 | 1 | 2 | 60 | NA |
| | NGG (SEQ ID NO: 94) | | | | | |
| SP598.CD5.g6 | GCTCTGGAGTTGTGGTGGGC | 1 | 1 | 4 | 74 | NA |
| | NGG (SEQ ID NO: 95) | | | | | |
| SP598.CD5.g4 | GTGGGCTCTGGAGTTGTGGT | 1 | 1 | 6 | 35 | NA |
| | NGG (SEQ ID NO: 96) | | | | | |
| SP598.CD5.g3 | TGTGGGCTCTGGAGTTGTGG | 1 | 1 | 8 | 54 | NA |
| | NGG (SEQ ID NO: 97) | | | | | |
| SP598.CD5.g13 | GTTGGAGGTGTTGTCTTCTGN | 1 | 2 | 2 | 48 | NA |
| | GG (SEQ ID NO: 98) | | | | | |
| SP598.CD5.g5 | GGCTCTGGAGTTGTGGTGGG | 1 | 3 | 9 | 51 | NA |
| | NGG (SEQ ID NO: 99) | | | | | |

TABLE 16
Guide RNA (gRNA) Off Target Analysis for hCD5 (Exon 5)

| Name | gRNA | long_0 | long_1 | long_2 | short_0 | SNP |
|---|---|---|---|---|---|---|
| SP599.CD5.g58 | CATAGCTGATGGTACCCCC | 1 | 1 | 1 | 1 | NA |
| | CNGG (SEQ ID NO: 100) | | | | | |
| SP599.CD5.g5 | CGGCCAGCACTGTGCCGGC | 1 | 1 | 1 | 2 | NA |
| | GNGG (SEQ ID NO: 101) | | | | | |
| SP599.CD5.g30 | CAAGAACTCGGCCACTTTT | 1 | 1 | 1 | 6 | NA |
| | CNGG (SEQ ID NO: 102) | | | | | |
| SP599.CD5.g44 | GGTGTTCCCGTGGCTCCCC | 1 | 1 | 1 | 11 | rs2241002:0.158 |
| | TNGG (SEQ ID NO: 103) | | | | | |
| SP599.CD5.g6 | CCAGCACTGTGCCGGCGTG | 1 | 1 | 1 | 13 | NA |
| | GNGG (SEQ ID NO: 104) | | | | | |
| SP599.CD5.g42 | GGCAAGGGCTGGTGTTCCC | 1 | 1 | 1 | 13 | NA |
| | GNGG (SEQ ID NO: 105) | | | | | |
| SP599.CD5.g7 | GGCGTGGTGGAGTTCTACA | 1 | 1 | 1 | 14 | NA |
| | GNGG (SEQ ID NO: 106) | | | | | |
| SP599.CD5.g60 | CCACCACGCCGGCACAGT | 1 | 1 | 1 | 15 | NA |
| | GCNGG (SEQ ID NO: 107) | | | | | |
| SP599.CD5.g8 | GGAGTTCTACAGCGGCAG | 1 | 1 | 1 | 17 | NA |
| | CCNGG (SEQ ID NO: 108) | | | | | |
| SP599.CD5.g11 | GTTCTACAGCGGCAGCCTG | 1 | 1 | 1 | 18 | NA |
| | GNGG (SEQ ID NO: 109) | | | | | |
| SP599.CD5.g25 | ACCAGCCCTTGCCAATCCA | 1 | 1 | 1 | 20 | NA |
| | ANGG (SEQ ID NO: 110) | | | | | |
| SP599.CD5.g10 | AGTTCTACAGCGGCAGCCT | 1 | 1 | 1 | 24 | NA |
| | GNGG (SEQ ID NO: 111) | | | | | |
| SP599.CD5.g55 | CCAGGTCCTGGGTCTTGTC | 1 | 1 | 1 | 25 | NA |
| | CNGG (SEQ ID NO: 112) | | | | | |
| SP599.CD5.g43 | TGGTGTTCCCGTGGCTCCC | 1 | 1 | 1 | 25 | rs2241002:0.158 |
| | CNGG (SEQ ID NO: 113) | | | | | |
| SP599.CD5.g9 | GAGTTCTACAGCGGCAGCC | 1 | 1 | 1 | 26 | NA |
| | TNGG (SEQ ID NO: 114) | | | | | |
| SP599.CD5.g26 | GAACTCAAGCTGTACCTCC | 1 | 1 | 1 | 29 | NA |
| | CNGG (SEQ ID NO: 115) | | | | | |
| SP599.CD5.g31 | AAGAACTCGGCCACTTTTC | 1 | 1 | 1 | 29 | NA |
| | TNGG (SEQ ID NO: 116) | | | | | |
| SP599.CD5.g41 | TCCATTGGATTGGCAAGGG | 1 | 1 | 1 | 32 | NA |
| | CNGG (SEQ ID NO: 117) | | | | | |
| SP599.CD5.g12 | TTCTACAGCGGCAGCCTGG | 1 | 1 | 1 | 33 | NA |
| | GNGG (SEQ ID NO: 118) | | | | | |
| SP599.CD5.g32 | AGAACTCGGCCACTTTTCT | 1 | 1 | 1 | 37 | NA |
| | GNGG (SEQ ID NO: 119) | | | | | |
| SP599.CD5.g49 | GCTTCAAGAAGGAGCCAC | 1 | 1 | 1 | 48 | NA |
| | ACNGG (SEQ ID NO: 120) | | | | | |
| SP599.CD5.g39 | GATCTTCCATTGGATTGGC | 1 | 1 | 2 | 7 | NA |
| | ANGG (SEQ ID NO: 121) | | | | | |
| SP599.CD5.g59 | GCTGTAGAACTCCACCACG | 1 | 1 | 2 | 11 | NA |
| | CNGG (SEQ ID NO: 122) | | | | | |
| SP599.CD5.g57 | GTCCTGGGCCTCATAGCTG | 1 | 1 | 2 | 13 | NA |
| | ANGG (SEQ ID NO: 123) | | | | | |
| SP599.CD5.g14 | TACCATCAGCTATGAGGCC | 1 | 1 | 2 | 14 | NA |
| | CNGG (SEQ ID NO: 124) | | | | | |
| SP599.CD5.g13 | GGGGGGTACCATCAGCTAT | 1 | 1 | 2 | 16 | NA |
| | GNGG (SEQ ID NO: 125) | | | | | |
| SP599.CD5.g35 | CCTGAAGCAATGCTCCAGG | 1 | 1 | 2 | 18 | NA |
| | GNGG (SEQ ID NO: 126) | | | | | |
| SP599.CD5.g33 | TTTTCCTGAAGCAATGCTC | 1 | 1 | 2 | 24 | NA |
| | CNGG (SEQ ID NO: 127) | | | | | |
| SP599.CD5.g48 | CTCTGGCAGATGCTTCAAG | 1 | 1 | 2 | 25 | NA |
| | ANGG (SEQ ID NO: 128) | | | | | |
| SP599.CD5.g53 | AGAGGAAGTTCTCCAGGTC | 1 | 1 | 2 | 53 | NA |
| | CNGG (SEQ ID NO: 129) | | | | | |
| SP599.CD5.g4 | TCTGGCGGCCAGCACTGTG | 1 | 1 | 2 | 166 | NA |
| | CNGG (SEQ ID NO: 130) | | | | | |
| SP599.CD5.g37 | TTGAGTTCTGGATCTTCCA | 1 | 1 | 3 | 9 | NA |
| | TNGG (SEQ ID NO: 131) | | | | | |
| SP599.CD5.g38 | TTCTGGATCTTCCATTGGA | 1 | 1 | 3 | 13 | NA |
| | TNGG (SEQ ID NO: 132) | | | | | |
| SP599.CD5.g40 | ATCTTCCATTGGATTGGCA | 1 | 1 | 3 | 18 | NA |
| | ANGG (SEQ ID NO: 133) | | | | | |
| SP599.CD5.g50 | TCAAGAAGGAGCCACACT | 1 | 1 | 3 | 31 | NA |
| | GGNGG (SEQ ID NO: 134) | | | | | |
| SP599.CD5.g36 | GGGAGGTACAGCTTGAGTT | 1 | 1 | 3 | 37 | NA |
| | CNGG (SEQ ID NO: 135) | | | | | |
| SP599.CD5.g45 | CCCGTGGCTCCCCTGGGTC | 1 | 1 | 3 | 43 | rs2241002:0.158 |
| | TNGG (SEQ ID NO: 136) | | | | | |
| SP599.CD5.g16 | CCAGGACAAGACCCAGGA | 1 | 1 | 3 | 57 | NA |
| | CCNGG (SEQ ID NO: 137) | | | | | |
| SP599.CD5.g17 | CTCTGCAACAACCTCCAGT | 1 | 1 | 3 | 67 | NA |
| | GNGG (SEQ ID NO: 138) | | | | | |
| SP599.CD5.g52 | TGTTGCAGAGGAAGTTCTC | 1 | 1 | 3 | 236 | NA |
| | CNGG (SEQ ID NO: 139) | | | | | |
| SP599.CD5.g56 | CAGGTCCTGGGTCTTGTCC | 1 | 1 | 4 | 24 | NA |
| | TNGG (SEQ ID NO: 140) | | | | | |
| SP599.CD5.g15 | TGAGGCCCAGGACAAGAC | 1 | 1 | 4 | 30 | NA |
| | CCNGG (SEQ ID NO: 141) | | | | | |
| SP599.CD5.g61 | CTGTGCCACCAGCTGCAGC | 1 | 1 | 4 | 133 | NA |
| | CNGG (SEQ ID NO: 142) | | | | | |
| SP599.CD5.g62 | TGTGCCACCAGCTGCAGCC | 1 | 1 | 4 | 139 | NA |
| | TNGG (SEQ ID NO: 143) | | | | | |
| SP599.CD5.g19 | CATCTGCCAGAGACTGAG | 1 | 1 | 4 | 1253 | NA |
| | GCNGG (SEQ ID NO: 144) | | | | | |
| SP599.CD5.g2 | CTGCAGCTGGTGGCACAGT | 1 | 1 | 5 | 17 | NA |
| | CNGG (SEQ ID NO: 145) | | | | | |
| SP599.CD5.g51 | CACACTGGAGGTTGTTGCA | 1 | 1 | 5 | 28 | NA |
| | GNGG (SEQ ID NO: 146) | | | | | |
| SP599.CD5.g3 | CAGCTGGTGGCACAGTCTG | 1 | 1 | 5 | 31 | NA |
| | GNGG (SEQ ID NO: 147) | | | | | |
| SP599.CD5.g29 | AGCAAAGGAGGGCAAGAA | 1 | 1 | 6 | 53 | NA |
| | CTNGG (SEQ ID NO: 148) | | | | | |
| SP599.CD5.g54 | GAGGAAGTTCTCCAGGTCC | 1 | 1 | 6 | 53 | NA |
| | TNGG (SEQ ID NO: 149) | | | | | |
| SP599.CD5.g63 | GCCACCAGCTGCAGCCTGG | 1 | 1 | 6 | 287 | NA |
| | GNGG (SEQ ID NO: 150) | | | | | |
| SP599.CD5.g20 | GCAGGCAGAGCCCAAGAC | 1 | 1 | 7 | 40 | rs2241002:0.158 |
| | CCNGG (SEQ ID NO: 151) | | | | | |
| SP599.CD5.g21 | CAGGCAGAGCCCAAGACC | 1 | 1 | 8 | 45 | rs2241002:0.158 |
| | CANGG (SEQ ID NO: 152) | | | | | |
| SP599.CD5.g1 | TCCTCCCAGGCTGCAGCTG | 1 | 1 | 8 | 140 | NA |
| | GNGG (SEQ ID NO: 153) | | | | | |
| SP599.CD5.g47 | GCTCTGCCTGCCTCAGTCT | 1 | 1 | 26 | 412 | NA |
| | CNGG (SEQ ID NO: 154) | | | | | |
| SP599.CD5.g27 | CCTCCCTGGAGCATTGCTT | 1 | 2 | 3 | 22 | NA |
| | CNGG (SEQ ID NO: 155) | | | | | |
| SP599.CD5.g34 | TTTCCTGAAGCAATGCTCC | 1 | 2 | 4 | 32 | NA |
| | ANGG (SEQ ID NO: 156) | | | | | |
| SP599.CD5.g46 | CCGTGGCTCCCCTGGGTCT | 1 | 2 | 5 | 37 | rs2241002:0.158 |
| | TNGG (SEQ ID NO: 157) | | | | | |
| SP599.CD5.g28 | AAAATCAAGCCCCAGAAA | 1 | 2 | 5 | 60 | NA |
| | AGNGG (SEQ ID NO: 158) | | | | | |
| SP599.CD5.g18 | GAAGCATCTGCCAGAGAC | 1 | 2 | 7 | 98 | NA |
| | TGNGG (SEQ ID NO: 159) | | | | | |
| SP599.CD5.g24 | CCAAGACCCAGGGGAGCC | 1 | 2 | 8 | 56 | rs2241002:0.158 |
| | ACNGG (SEQ ID NO: 160) | | | | | |
| SP599.CD5.g22 | AGGCAGAGCCCAAGACCC | 1 | 2 | 10 | 41 | rs2241002:0.158 |
| | AGNGG (SEQ ID NO: 161) | | | | | |
| SP599.CD5.g23 | CCCAAGACCCAGGGGAGC | 1 | 2 | 10 | 99 | rs2241002:0.158 |
| | CANGG (SEQ ID NO: 162) | | | | | |

The gRNA sequences in Table 14, Table 15, and Table 16 were normalized (% Normalization to NHEJ) for gRNA activity via next generation sequencing (NGS). GFP was used as a control. Following sequencing analysis, the following gRNAs were recommended based on off-target profile: Exon 3: SP597.hCD5.g2 (76.5%), SP597.hCD5.g22 (36.3%), SP597.hCD5.g39 (16.0%), SP597.hCD5.g46. Exon4: SP598.hCD5.g7, SP598.hCD5.g10 (58.5%). Exon5: SP599.hCD5.g5 (51.0%), SP599.hCD5.g30, SP599.hCD5.g42, SP599.hCD5.g58 (41.0%)

hCSF2.

Off target analysis of selected gRNA was performed for hCSF2 to determine the number of sites in human genome which are an exact match or contains up to 1 or 2 mismatches, which may include the target site. The results are listed in Table 17 for hCSF2.

TABLE 17
Guide RNA (gRNA) Off Target Analysis for hCSF2

| Name | gRNA | long_0 | long_1 | long_2 | long_3 | short_0 | SNP |
|---|---|---|---|---|---|---|---|
| MS1086.CSF2.sp8 | TACTCAGGTTCAGGAGA | 1 | 1 | 1 | 10 | 11 | NA |
| | CGCNGG (SEQ ID NO: 163) | | | | | | |
| MS1086.CSF2.sp10 | TCAGGAGACGCCGGGCC | 1 | 1 | 1 | 20 | 38 | NA |
| | TCCNGG (SEQ ID NO: 164) | | | | | | |
| MS1086.CSF2.sp9 | ACTCAGGTTCAGGAGAC | 1 | 1 | 1 | 20 | 16 | NA |
| | GCCNGG (SEQ ID NO: 165) | | | | | | |
| MS1086.CSF2.sp7 | CAGTGTCTCTACTCAGGT | 1 | 1 | 2 | 22 | 29 | NA |
| | TCNGG (SEQ ID NO: 166) | | | | | | |
| MS1086.CSF2.sp14 | ATGCTCCCAGGGCTGCGT | 1 | 1 | 2 | 42 | 34 | rs2069622 |
| | GCNGG (SEQ ID NO: 167) | | | | | | |
| MS1086.CSF2.sp11 | GAGACGCCGGGCCTCCT | 1 | 1 | 2 | 26 | 146 | NA |
| | GGANGG (SEQ ID NO: 168) | | | | | | |
| MS1086.CSF2.sp6 | CAGCAGCAGTGTCTCTAC | 1 | 1 | 3 | 39 | 24 | NA |
| | TCNGG (SEQ ID NO: 169) | | | | | | |
| MS1086.CSF2.sp12 | GATGGCATTCACATGCTC | 1 | 1 | 3 | 28 | 59 | NA |
| | CCNGG (SEQ ID NO: 170) | | | | | | |
| MS1086.CSF2.sp2 | GGAGCATGTGAATGCCA | 1 | 1 | 3 | 26 | 48 | NA |
| | TCCNGG (SEQ ID NO: 171) | | | | | | |
| MS1086.CSF2.sp5 | TAGAGACACTGCTGCTG | 1 | 1 | 3 | 56 | 168 | NA |
| | AGANGG (SEQ ID NO: 172) | | | | | | |
| MS1086.CSF2.sp3 | GCATGTGAATGCCATCCA | 1 | 1 | 3 | 41 | 56 | NA |
| | GGNGG (SEQ ID NO: 173) | | | | | | |
| MS1086.CSF2.sp13 | ATGGCATTCACATGCTCC | 1 | 1 | 4 | 30 | 80 | NA |
| | CANGG (SEQ ID NO: 174) | | | | | | |
| MS1086.CSF2.sp4 | TGAATGCCATCCAGGAG | 1 | 1 | 5 | 65 | 180 | NA |
| | GCCNGG (SEQ ID NO: 175) | | | | | | |
| MS1086.CSF2.sp15 | TGCTCCCAGGGCTGCGTG | 1 | 1 | 6 | 57 | 29 | rs2069622 |
| | CTNGG (SEQ ID NO: 176) | | | | | | |
| MS1086.CSF2.sp1 | CAGCCCCAGCACGCAGC | 1 | 1 | 15 | 146 | 41 | rs2069622 |
| | CCTNGG (SEQ ID NO: 177) | | | | | | |
| MS1086.CSF2.sp16 | GCTCCCAGGGCTGCGTGC | 1 | 2 | 9 | 85 | 37 | rs2069622 |
| | TGNGG (SEQ ID NO: 178) | | | | | | |

The gRNA sequences in Table 17 were normalized (% Normalization to NHEJ) for gRNA activity via next generation sequencing (NGS). GFP was used as a control. Following sequencing analysis, the following gRNAs were recommended based on off-target profile: MS1086.CSF2.sp8 (>15%) and MS1086.CSF2.sp10 (>15%).

hCTLA4.

Off target analysis of selected gRNA was performed for 2 exons of hCTLA4 (Exon 1 and Exon 2) to determine the number of sites in human genome which are an exact match or contains up to 1 or 2 mismatches, which may include the target site. The results are listed in Table 18 for Exon 1 and Table 19 for Exon 2 for hCTLA4.

TABLE 18
Guide RNA (gRNA) Off Target Analysis for hCTLA4 (Exon 1)

| Name | gRNA | long_0 | long_1 | long_2 | short_0 | SNP |
|---|---|---|---|---|---|---|
| SP621.CTLA4.g2 | CCTTGGATTTCAGCGGCAC | 1 | 1 | 1 | 5 | NA |
| | ANGG (SEQ ID NO: 179) | | | | | |
| SP621.CTLA4.g12 | CCTTGTGCCGCTGAAATCC | 1 | 1 | 1 | 5 | NA |
| | ANGG (SEQ ID NO: 180) | | | | | |
| SP621.CTLA4.g5 | TGAACCTGGCTACCAGGA | 1 | 1 | 1 | 11 | rs231775:0.452 |
| | CCNGG (SEQ ID NO: 181) | | | | | |
| SP621.CTLA4.g11 | AGGGCCAGGTCCTGGTAG | 1 | 1 | 3 | 16 | rs231775:0.452 |
| | CCNGG (SEQ ID NO: 182) | | | | | |
| SP621.CTLA4.g4 | CTCAGCTGAACCTGGCTAC | 1 | 1 | 3 | 17 | rs231775:0.452 |
| | CNGG (SEQ ID NO: 183) | | | | | |
| SP621.CTLA4.g8 | AGAAAAAACAGGAGAGTG | 1 | 1 | 3 | 39 | NA |
| | CANGG (SEQ ID NO: 184) | | | | | |
| SP621.CTLA4.g3 | GCACAAGGCTCAGCTGAA | 1 | 1 | 4 | 29 | NA |
| | CCNGG (SEQ ID NO: 185) | | | | | |
| SP621.CTLA4.g1 | TGGCTTGCCTTGGATTTCA | 1 | 1 | 6 | 33 | NA |
| | GNGG (SEQ ID NO: 186) | | | | | |
| SP621.CTLA4.g9 | AAACAGGAGAGTGCAGGG | 1 | 1 | 6 | 69 | NA |
| | CCNGG (SEQ ID NO: 187) | | | | | |
| SP621.CTLA4.g10 | GAGAGTGCAGGGCCAGGT | 1 | 1 | 7 | 50 | NA |
| | CCNGG (SEQ ID NO: 188) | | | | | |
| SP621.CTLA4.g6 | GGATGAAGAGAAGAAAAA | 1 | 1 | 8 | 173 | NA |
| | ACNGG (SEQ ID NO: 189) | | | | | |
| SP621.CTLA4.g7 | AAGAAAAAACAGGAGAGT | 1 | 2 | 8 | 33 | NA |
| | GCNGG (SEQ ID NO: 190) | | | | | |

TABLE 19
Guide RNA (gRNA) Off Target Analysis for hCTLA4 (Exon 2)

| Name | gRNA | long_0 | long_1 | long_2 | short_0 | SNP |
|---|---|---|---|---|---|---|
| SP622.CTLA4.g9 | CCGGGTGACAGTGCTTCGGC | 1 | 1 | 1 | 2 | NA |
| | NGG (SEQ ID NO: 191) | | | | | |
| SP622.CTLA4.g33 | ACACAAAGCTGGCGATGCC | 1 | 1 | 1 | 4 | NA |
| | TNGG (SEQ ID NO: 192) | | | | | |
| SP622.CTLA4.g21 | CCCTCAGTCCTTGGATAGTG | 1 | 1 | 1 | 8 | NA |
| | NGG (SEQ ID NO: 193) | | | | | |
| SP622.CTLA4.g14 | GTGCGGCAACCTACATGATG | 1 | 1 | 1 | 9 | NA |
| | NGG (SEQ ID NO: 194) | | | | | |
| SP622.CTLA4.g12 | CTGTGCGGCAACCTACATGA | 1 | 1 | 1 | 13 | NA |
| | NGG (SEQ ID NO: 195) | | | | | |
| SP622.CTLA4.g2 | GGCCCAGCCTGCTGTGGTAC | 1 | 1 | 1 | 17 | NA |
| | NGG (SEQ ID NO: 196) | | | | | |
| SP622.CTLA4.g23 | GTTCACTTGATTTCCACTGG | 1 | 1 | 1 | 17 | NA |
| | NGG (SEQ ID NO: 197) | | | | | |
| SP622.CTLA4.g27 | CAACTCATTCCCCATCATGT | 1 | 1 | 1 | 18 | NA |
| | NGG (SEQ ID NO: 198) | | | | | |
| SP622.CTLA4.g28 | CCGCACAGACTTCAGTCACC | 1 | 1 | 1 | 20 | NA |
| | NGG (SEQ ID NO: 199) | | | | | |
| SP622.CTLA4.g13 | TGTGCGGCAACCTACATGAT | 1 | 1 | 1 | 30 | NA |
| | NGG (SEQ ID NO: 200) | | | | | |
| SP622.CTLA4.g20 | CCTCACTATCCAAGGACTGA | 1 | 1 | 1 | 30 | NA |
| | NGG (SEQ ID NO: 201) | | | | | |
| SP622.CTLA4.g31 | CGGACCTCAGTGGCTTTGCC | 1 | 1 | 1 | 34 | NA |
| | NGG (SEQ ID NO: 202) | | | | | |
| SP622.CTLA4.g22 | GAGGTTCACTTGATTTCCAC | 1 | 1 | 1 | 40 | NA |
| | NGG (SEQ ID NO: 203) | | | | | |
| SP622.CTLA4.g11 | CCAGGTGACTGAAGTCTGTG | 1 | 1 | 1 | 45 | NA |
| | NGG (SEQ ID NO: 204) | | | | | |
| SP622.CTLA4.g24 | ACTGGAGGTGCCCGTGCAG | 1 | 1 | 2 | 15 | NA |
| | ANGG (SEQ ID NO: 205) | | | | | |
| SP622.CTLA4.g18 | CAAGTGAACCTCACTATCCA | 1 | 1 | 2 | 16 | NA |
| | NGG (SEQ ID NO: 206) | | | | | |
| SP622.CTLA4.g3 | GTGGTACTGGCCAGCAGCC | 1 | 1 | 2 | 29 | NA |
| | GNGG (SEQ ID NO: 207) | | | | | |
| SP622.CTLA4.g8 | AGGTCCGGGTGACAGTGCTT | 1 | 1 | 2 | 29 | NA |
| | NGG (SEQ ID NO: 208) | | | | | |
| SP622.CTLA4.g17 | ATCTGCACGGGCACCTCCAG | 1 | 1 | 2 | 29 | NA |
| | NGG (SEQ ID NO: 209) | | | | | |
| SP622.CTLA4.g25 | CCGTGCAGATGGAATCATCT | 1 | 1 | 2 | 36 | NA |
| | NGG (SEQ ID NO: 210) | | | | | |
| SP622.CTLA4.g16 | CTAGATGATTCCATCTGCAC | 1 | 1 | 2 | 39 | NA |
| | NGG (SEQ ID NO: 211) | | | | | |
| SP622.CTLA4.g19 | ACCTCACTATCCAAGGACTG | 1 | 1 | 2 | 40 | NA |
| | NGG (SEQ ID NO: 212) | | | | | |
| SP622.CTLA4.g29 | CCTGCCGAAGCACTGTCACC | 1 | 1 | 2 | 47 | NA |
| | NGG (SEQ ID NO: 213) | | | | | |
| SP622.CTLA4.g36 | TGGCCAGTACCACAGCAGG | 1 | 1 | 2 | 74 | NA |
| | CNGG (SEQ ID NO: 214) | | | | | |
| SP622.CTLA4.g5 | ATCTCCAGGCAAAGCCACTG | 1 | 1 | 2 | 80 | NA |
| | NGG (SEQ ID NO: 215) | | | | | |
| SP622.CTLA4.g1 | GCACGTGGCCCAGCCTGCTG | 1 | 1 | 2 | 121 | NA |
| | NGG (SEQ ID NO: 216) | | | | | |
| SP622.CTLA4.g4 | GTGTGTGAGTATGCATCTCC | 1 | 1 | 3 | 8 | NA |
| | NGG (SEQ ID NO: 217) | | | | | |
| SP622.CTLA4.g30 | CACTGTCACCCGGACCTCAG | 1 | 1 | 3 | 9 | NA |
| | NGG (SEQ ID NO: 218) | | | | | |
| SP622.CTLA4.g34 | GCTGGCGATGCCTCGGCTGC | 1 | 1 | 3 | 17 | NA |
| | NGG (SEQ ID NO: 219) | | | | | |
| SP622.CTLA4.g35 | CTGCTGGCCAGTACCACAGC | 1 | 1 | 3 | 22 | NA |
| | NGG (SEQ ID NO: 220) | | | | | |
| SP622.CTLA4.g7 | AGGCAAAGCCACTGAGGTC | 1 | 1 | 3 | 40 | NA |
| | CNGG (SEQ ID NO: 221) | | | | | |
| SP622.CTLA4.g26 | GCAGATGGAATCATCTAGG | 1 | 1 | 4 | 20 | NA |
| | ANGG (SEQ ID NO: 222) | | | | | |
| SP622.CTLA4.g15 | CCTAGATGATTCCATCTGCA | 1 | 1 | 4 | 40 | NA |
| | NGG (SEQ ID NO: 223) | | | | | |
| SP622.CTLA4.g37 | GGCCAGTACCACAGCAGGC | 1 | 1 | 4 | 65 | NA |
| | TNGG (SEQ ID NO: 224) | | | | | |
| SP622.CTLA4.g32 | TGCATACTCACACACAAAGC | 1 | 1 | 7 | 71 | NA |
| | NGG (SEQ ID NO: 225) | | | | | |
| SP622.CTLA4.g10 | GCTTCGGCAGGCTGACAGCC | 1 | 1 | 8 | 58 | NA |
| | NGG (SEQ ID NO: 226) | | | | | |
| SP622.CTLA4.g6 | CAGGCAAAGCCACTGAGGT | 1 | 1 | 11 | 30 | NA |
| | CNGG (SEQ ID NO: 227) | | | | | |

The gRNA sequences in Table 18 and Table 19 were normalized (% Normalization to NHEJ) for gRNA activity via next generation sequencing (NGS). GFP was used as a control. Following sequencing analysis, the following gRNAs were recommended based on off-target profile: Exon 1: SP621.hCTLA4.g2 (>15%) and SP621.hCTLA4.g12 (>15%). Exon 2: SP622.hCTLA4.g2 (>15%), SP622.hCTLA4.g9 (>15%), and SP622.hCTLA4.g33 (>15%).

hPDCD1.

Off target analysis of selected gRNA was performed for 2 exons of hPDCD1 (CF60 and CF61) to determine the number of sites in human genome which are an exact match or contains up to 1 or 2 mismatches, which may include the target site. The results are listed in Table 20 for Exon CF60 and Table 21 for Exon CF61.

TABLE 20
Guide RNA (gRNA) Off Target Analysis for hPDCD1 (Exon CF60)

| Name | gRNA | long_0 | long_1 | long_2 | short_0 | SNP |
|---|---|---|---|---|---|---|
| CF60.PDCD1.g12 | TGTAGCACCGCCCAGACGAC | 1 | 1 | 1 | 1 | NA |
| | NGG (SEQ ID NO: 228) | | | | | |
| CF60.PDCD1.g3 | GGCGCCCTGGCCAGTCGTCT | 1 | 1 | 1 | 3 | NA |
| | NGG (SEQ ID NO: 229) | | | | | |
| CF60.PDCD1.g5 | CGTCTGGGCGGTGCTACAAC | 1 | 1 | 1 | 3 | NA |
| | NGG (SEQ ID NO: 230) | | | | | |
| CF60.PDCD1.g2 | AGGCGCCCTGGCCAGTCGTC | 1 | 1 | 1 | 5 | NA |
| | NGG (SEQ ID NO: 231) | | | | | |
| CF60.PDCD1.g13 | CACCGCCCAGACGACTGGCC | 1 | 1 | 1 | 5 | NA |
| | NGG (SEQ ID NO: 232) | | | | | |
| CF60.PDCD1.g14 | ACCGCCCAGACGACTGGCCA | 1 | 1 | 1 | 5 | NA |
| | NGG (SEQ ID NO: 233) | | | | | |
| CF60.PDCD1.g7 | GGGCGGTGCTACAACTGGGC | 1 | 1 | 1 | 7 | NA |
| | NGG (SEQ ID NO: 234) | | | | | |
| CF60.PDCD1.g6 | GTCTGGGCGGTGCTACAACT | 1 | 1 | 1 | 9 | NA |
| | NGG (SEQ ID NO: 235) | | | | | |
| CF60.PDCD1.g16 | CGACTGGCCAGGGCGCCTGT | 1 | 1 | 1 | 15 | NA |
| | NGG (SEQ ID NO: 236) | | | | | |
| CF60.PDCD1.g8 | CGGTGCTACAACTGGGCTGG | 1 | 1 | 1 | 33 | NA |
| | NGG (SEQ ID NO: 237) | | | | | |
| CF60.PDCD1.g11 | TGGCGGCCAGGATGGTTCTT | 1 | 1 | 1 | 33 | NA |
| | NGG (SEQ ID NO: 238) | | | | | |
| CF60.PDCD1.g15 | ACGACTGGCCAGGGCGCCTG | 1 | 1 | 1 | 45 | NA |
| | NGG (SEQ ID NO: 239) | | | | | |
| CF60.PDCD1.g9 | CTACAACTGGGCTGGCGGCC | 1 | 1 | 1 | 57 | NA |
| | NGG (SEQ ID NO: 240) | | | | | |
| CF60.PDCD1.g4 | GCCCTGGCCAGTCGTCTGGG | 1 | 1 | 2 | 2 | NA |
| | NGG (SEQ ID NO: 241) | | | | | |
| CF60.PDCD1.g1 | TGCAGATCCCACAGGCGCCC | 1 | 1 | 2 | 23 | NA |
| | NGG (SEQ ID NO: 242) | | | | | |
| CF60.PDCD1.g10 | AACTGGGCTGGCGGCCAGG | 1 | 1 | 3 | 17 | NA |
| | ANGG (SEQ ID NO: 243) | | | | | |

TABLE 21
Guide RNA (gRNA) Off Target Analysis for hPDCD1 (CF61)

| Name | gRNA | long_0 | long_1 | long_2 | short_0 | SNP |
|---|---|---|---|---|---|---|
| CF61.PDCD1.g6 | CGGAGAGCTTCGTGCTAAAC | 1 | 1 | 1 | 1 | NA |
| | NGG (SEQ ID NO: 244) | | | | | |
| CF61.PDCD1.g14 | GCGTGACTTCCACATGAGCG | 1 | 1 | 1 | 2 | NA |
| | NGG (SEQ ID NO: 245) | | | | | |
| CF61.PDCD1.g17 | ATGTGGAAGTCACGCCCGTT | 1 | 1 | 1 | 2 | NA |
| | NGG (SEQ ID NO: 246) | | | | | |
| CF61.PDCD1.g2 | GCCCTGCTCGTGGTGACCGA | 1 | 1 | 1 | 3 | NA |
| | NGG (SEQ ID NO: 247) | | | | | |
| CF61.PDCD1.g35 | CACGAAGCTCTCCGATGTGT | 1 | 1 | 1 | 3 | NA |
| | NGG (SEQ ID NO: 248) | | | | | |
| CF61.PDCD1.g4 | CCTGCTCGTGGTGACCGAAG | 1 | 1 | 1 | 4 | NA |
| | NGG (SEQ ID NO: 249) | | | | | |
| CF61.PDCD1.g20 | TGACACGGAAGCGGCAGTCC | 1 | 1 | 1 | 5 | NA |
| | NGG (SEQ ID NO: 250) | | | | | |
| CF61.PDCD1.g40 | CCCCTTCGGTCACCACGAGC | 1 | 1 | 1 | 5 | NA |
| | NGG (SEQ ID NO: 251) | | | | | |
| CF61.PDCD1.g8 | CAGCAACCAGACGGACAAG | 1 | 1 | 1 | 6 | NA |
| | CNGG (SEQ ID NO: 252) | | | | | |
| CF61.PDCD1.g19 | GCAGTTGTGTGACACGGAAG | 1 | 1 | 1 | 6 | NA |
| | NGG (SEQ ID NO: 253) | | | | | |
| CF61.PDCD1.g41 | CCCTTCGGTCACCACGAGCA | 1 | 1 | 1 | 6 | NA |
| | NGG (SEQ ID NO: 254) | | | | | |
| CF61.PDCD1.g26 | CCGGGCTGGCTGCGGTCCTC | 1 | 1 | 1 | 8 | NA |
| | NGG (SEQ ID NO: 255) | | | | | |
| CF61.PDCD1.g30 | AGGCGGCCAGCTTGTCCGTC | 1 | 1 | 1 | 8 | NA |
| | NGG (SEQ ID NO: 256) | | | | | |
| CF61.PDCD1.g31 | CAGCTTGTCCGTCTGGTTGC | 1 | 1 | 1 | 8 | NA |
| | NGG (SEQ ID NO: 257) | | | | | |
| CF61.PDCD1.g43 | CGGTCACCACGAGCAGGGCT | 1 | 1 | 1 | 10 | NA |
| | NGG (SEQ ID NO: 258) | | | | | |
| CF61.PDCD1.g13 | GTGTCACACAACTGCCCAAC | 1 | 1 | 1 | 13 | NA |
| | NGG (SEQ ID NO: 259) | | | | | |
| CF61.PDCD1.g5 | CTGCAGCTTCTCCAACACAT | 1 | 1 | 1 | 23 | NA |
| | NGG (SEQ ID NO: 260) | | | | | |
| CF61.PDCD1.g9 | CAAGCTGGCCGCCTTCCCCG | 1 | 1 | 1 | 23 | NA |
| | NGG (SEQ ID NO: 261) | | | | | |
| CF61.PDCD1.g12 | CGTGTCACACAACTGCCCAA | 1 | 1 | 1 | 28 | NA |
| | NGG (SEQ ID NO: 262) | | | | | |
| CF61.PDCD1.g18 | CGTTGGGCAGTTGTGTGACA | 1 | 1 | 1 | 32 | NA |
| | NGG (SEQ ID NO: 263) | | | | | |
| CF61.PDCD1.g33 | GCTTGTCCGTCTGGTTGCTGN | 1 | 1 | 1 | 41 | NA |
| | GG (SEQ ID NO: 264) | | | | | |
| CF61.PDCD1.g22 | CGGAAGCGGCAGTCCTGGCC | 1 | 1 | 1 | 61 | NA |
| | NGG (SEQ ID NO: 265) | | | | | |
| CF61.PDCD1.g36 | CGATGTGTTGGAGAAGCTGC | 1 | 1 | 1 | 135 | NA |
| | NGG (SEQ ID NO: 266) | | | | | |
| CF61.PDCD1.g16 | CATGTGGAAGTCACGCCCGT | 1 | 1 | 2 | 2 | NA |
| | NGG (SEQ ID NO: 267) | | | | | |
| CF61.PDCD1.g3 | CCCTGCTCGTGGTGACCGAA | 1 | 1 | 2 | 3 | NA |
| | NGG (SEQ ID NO: 268) | | | | | |
| CF61.PDCD1.g27 | CGGGCTGGCTGCGGTCCTCG | 1 | 1 | 2 | 3 | NA |
| | NGG (SEQ ID NO: 269) | | | | | |
| CF61.PDCD1.g32 | AGCTTGTCCGTCTGGTTGCTN | 1 | 1 | 2 | 4 | NA |
| | GG (SEQ ID NO: 270) | | | | | |
| CF61.PDCD1.g39 | GAAGGTGGCGTTGTCCCCTT | 1 | 1 | 2 | 4 | NA |
| | NGG (SEQ ID NO: 271) | | | | | |
| CF61.PDCD1.g15 | ACTTCCACATGAGCGTGGTC | 1 | 1 | 2 | 6 | NA |
| | NGG (SEQ ID NO: 272) | | | | | |
| CF61.PDCD1.g25 | GCCGGGCTGGCTGCGGTCCT | 1 | 1 | 2 | 17 | NA |
| | NGG (SEQ ID NO: 273) | | | | | |
| CF61.PDCD1.g42 | TCGGTCACCACGAGCAGGGC | 1 | 1 | 2 | 23 | NA |
| | NGG (SEQ ID NO: 274) | | | | | |
| CF61.PDCD1.g34 | TCTGGTTGCTGGGGCTCATG | 1 | 1 | 2 | 31 | NA |
| | NGG (SEQ ID NO: 275) | | | | | |
| CF61.PDCD1.g21 | ACGGAAGCGGCAGTCCTGGC | 1 | 1 | 2 | 41 | NA |
| | NGG (SEQ ID NO: 276) | | | | | |
| CF61.PDCD1.g10 | CCCGAGGACCGCAGCCAGCC | 1 | 1 | 2 | 46 | NA |
| | NGG (SEQ ID NO: 277) | | | | | |
| CF61.PDCD1.g28 | CTGGCTGCGGTCCTCGGGGA | 1 | 1 | 3 | 16 | NA |
| | NGG (SEQ ID NO: 278) | | | | | |
| CF61.PDCD1.g7 | CATGAGCCCCAGCAACCAGA | 1 | 1 | 3 | 33 | NA |
| | NGG (SEQ ID NO: 279) | | | | | |
| CF61.PDCD1.g24 | AGTCCTGGCCGGGCTGGCTG | 1 | 1 | 3 | 42 | NA |
| | NGG (SEQ ID NO: 280) | | | | | |
| CF61.PDCD1.g55 | GGGGGTTCCAGGGCCTGTCT | 1 | 1 | 3 | 126 | NA |
| | NGG (SEQ ID NO: 281) | | | | | |
| CF61.PDCD1.g44 | GGTCACCACGAGCAGGGCTG | 1 | 1 | 4 | 26 | NA |
| | NGG (SEQ ID NO: 282) | | | | | |
| CF61.PDCD1.g29 | GCTGCGGTCCTCGGGGAAGG | 1 | 1 | 4 | 35 | NA |
| | NGG (SEQ ID NO: 283) | | | | | |
| CF61.PDCD1.g11 | GGACCGCAGCCAGCCCGGCC | 1 | 1 | 4 | 47 | NA |
| | NGG (SEQ ID NO: 284) | | | | | |
| CF61.PDCD1.g53 | GAGAAGGTGGGGGGGTTCCA | 1 | 1 | 5 | 8 | NA |
| | NGG (SEQ ID NO: 285) | | | | | |
| CF61.PDCD1.g52 | GGAGAAGGTGGGGGGGTTCC | 1 | 1 | 5 | 15 | NA |
| | NGG (SEQ ID NO: 286) | | | | | |
| CF61.PDCD1.g23 | AGCGGCAGTCCTGGCCGGGC | 1 | 1 | 5 | 39 | NA |
| | NGG (SEQ ID NO: 287) | | | | | |
| CF61.PDCD1.g56 | GGGGTTCCAGGGCCTGTCTG | 1 | 1 | 5 | 97 | NA |
| | NGG (SEQ ID NO: 288) | | | | | |
| CF61.PDCD1.g1 | CTTCTCCCCAGCCCTGCTCGN | 1 | 1 | 6 | 22 | NA |
| | GG (SEQ ID NO: 289) | | | | | |
| CF61.PDCD1.g37 | GTTGGAGAAGCTGCAGGTGA | 1 | 1 | 6 | 88 | NA |
| | NGG (SEQ ID NO: 290) | | | | | |
| CF61.PDCD1.g54 | GGGGGGTTCCAGGGCCTGTC | 1 | 1 | 6 | 1286 | NA |
| | NGG (SEQ ID NO: 291) | | | | | |
| CF61.PDCD1.g38 | GGAGAAGCTGCAGGTGAAG | 1 | 1 | 9 | 66 | NA |
| | GNGG (SEQ ID NO: 292) | | | | | |
| CF61.PDCD1.g45 | CACGAGCAGGGCTGGGGAG | 1 | 1 | 10 | 448 | NA |
| | ANGG (SEQ ID NO: 293) | | | | | |
| CF61.PDCD1.g48 | GCAGGGCTGGGGAGAAGGT | 1 | 1 | 21 | 125 | NA |
| | GNGG (SEQ ID NO: 294) | | | | | |
| CF61.PDCD1.g49 | CAGGGCTGGGGAGAAGGTG | 1 | 1 | 29 | 214 | NA |
| | GNGG (SEQ ID NO: 295) | | | | | |
| CF61.PDCD1.g46 | GAGCAGGGCTGGGGAGAAG | 1 | 1 | 30 | 202 | NA |
| | GNGG (SEQ ID NO: 296) | | | | | |
| CF61.PDCD1.g47 | AGCAGGGCTGGGGAGAAGG | 1 | 2 | 11 | 136 | NA |
| | TNGG (SEQ ID NO: 297) | | | | | |
| CF61.PDCD1.g50 | AGGGCTGGGGAGAAGGTGG | 1 | 2 | 31 | 179 | NA |
| | GNGG (SEQ ID NO: 298) | | | | | |
| CF61.PDCD1.g51 | GGGCTGGGGAGAAGGTGGG | 1 | 2 | 49 | 130 | NA |
| | GNGG (SEQ ID NO: 299) | | | | | |

The gRNA sequences in Table 20 and Table 21 were normalized (% Normalization to NHEJ) for gRNA activity via next generation sequencing (NGS). GFP was used as a control. Following sequencing analysis, the following gRNAs were recommended based on off-target profile: CF60.PDCD1.g12 (65.6%), CF60.PDCD1.g3 (69.2%), CF61.PDCD1.g6, CF61.PDCD1.g2 (72.7%), and CF61.PDCD1.g35 (24.0%).

hTIM3.

Off target analysis of selected gRNA was performed for 2 exons of hTIM3 (Exon 2 and Exon 3) to determine the number of sites in human genome which are an exact match or contains up to 1 or 2 mismatches, which may include the target site. The results are listed in Table 22 for Exon 2 and Table 23 for Exon 3.

TABLE 22
Guide RNA (gRNA) Off Target Analysis for hTIM3 (Exon 2)

| Name | gRNA | long_0 | long_1 | long_2 | short_0 | SNP |
|---|---|---|---|---|---|---|
| SP619.TIM3.g2 | AGAAGTGGAATACAGAGCGG | 1 | 1 | 1 | 2 | NA |
| | NGG (SEQ ID NO: 300) | | | | | |
| SP619.TIM3.g12 | AATGTGGCAACGTGGTGCTC | 1 | 1 | 1 | 3 | NA |
| | NGG (SEQ ID NO: 301) | | | | | |
| SP619.TIM3.g20 | CTAAATGGGGATTTCCGCAA | 1 | 1 | 1 | 4 | NA |
| | NGG (SEQ ID NO: 302) | | | | | |
| SP619.TIM3.g18 | CATCCAGATACTGGCTAAAT | 1 | 1 | 1 | 8 | NA |
| | NGG (SEQ ID NO: 303) | | | | | |
| SP619.TIM3.g41 | CAGACGGGCACGAGGTTCCC | 1 | 1 | 1 | 8 | NA |
| | NGG (SEQ ID NO: 304) | | | | | |
| SP619.TIM3.g49 | GCGGCTGGGGTGTAGAAGCA | 1 | 1 | 1 | 8 | NA |
| | NGG (SEQ ID NO: 305) | | | | | |
| SP619.TIM3.g7 | GAACCTCGTGCCCGTCTGCT | 1 | 1 | 1 | 10 | NA |
| | NGG (SEQ ID NO: 306) | | | | | |
| SP619.TIM3.g43 | GACGGGCACGAGGTTCCCTG | 1 | 1 | 1 | 10 | NA |
| | NGG (SEQ ID NO: 307) | | | | | |
| SP619.TIM3.g35 | ATCCCCATTTAGCCAGTATCN | 1 | 1 | 1 | 11 | NA |
| | GG (SEQ ID NO: 308) | | | | | |
| SP619.TIM3.g3 | GTGGAATACAGAGCGGAGGT | 1 | 1 | 1 | 12 | NA |
| | NGG (SEQ ID NO: 309) | | | | | |
| SP619.TIM3.g42 | AGACGGGCACGAGGTTCCCT | 1 | 1 | 1 | 12 | NA |
| | NGG (SEQ ID NO: 310) | | | | | |
| SP619.TIM3.g6 | GGAACCTCGTGCCCGTCTGC | 1 | 1 | 1 | 13 | NA |
| | NGG (SEQ ID NO: 311) | | | | | |
| SP619.TIM3.g32 | GAGTCACATTCTCTATGGTCN | 1 | 1 | 1 | 14 | NA |
| | GG (SEQ ID NO: 312) | | | | | |
| SP619.TIM3.g22 | ATGTGACTCTAGCAGACAGT | 1 | 1 | 1 | 16 | NA |
| | NGG (SEQ ID NO: 313) | | | | | |
| SP619.TIM3.g27 | TTTTCATCATTCATTATGCCN | 1 | 1 | 1 | 16 | NA |
| | GG (SEQ ID NO: 314) | | | | | |
| SP619.TIM3.g21 | AATGTGACTCTAGCAGACAG | 1 | 1 | 1 | 17 | NA |
| | NGG (SEQ ID NO: 315) | | | | | |
| SP619.TIM3.g19 | ATCCAGATACTGGCTAAATG | 1 | 1 | 1 | 18 | NA |
| | NGG (SEQ ID NO: 316) | | | | | |
| SP619.TIM3.g24 | TGCTGCCGGATCCAAATCCC | 1 | 1 | 1 | 22 | NA |
| | NGG (SEQ ID NO: 317) | | | | | |
| SP619.TIM3.g5 | TCTACACCCCAGCCGCCCCA | 1 | 1 | 1 | 30 | NA |
| | NGG (SEQ ID NO: 318) | | | | | |
| SP619.TIM3.g30 | TTATGCCTGGGATTTGGATCN | 1 | 1 | 1 | 35 | NA |
| | GG (SEQ ID NO: 319) | | | | | |
| SP619.TIM3.g51 | CGCTCTGTATTCCACTTCTGN | 1 | 1 | 1 | 83 | NA |
| | GG (SEQ ID NO: 320) | | | | | |
| SP619.TIM3.g47 | GAGGTTCCCTGGGGCGGCTG | 1 | 1 | 1 | 85 | NA |
| | NGG (SEQ ID NO: 321) | | | | | |
| SP619.TIM3.g40 | TGCCCCAGCAGACGGGCACG | 1 | 1 | 2 | 5 | NA |
| | NGG (SEQ ID NO: 322) | | | | | |
| SP619.TIM3.g23 | ACAGTGGGATCTACTGCTGC | 1 | 1 | 2 | 8 | NA |
| | NGG (SEQ ID NO: 323) | | | | | |
| SP619.TIM3.g11 | TGTGTTTGAATGTGGCAACG | 1 | 1 | 2 | 9 | NA |
| | NGG (SEQ ID NO: 324) | | | | | |
| SP619.TIM3.g25 | TGAAAAATTTAACCTGAAGT | 1 | 1 | 2 | 16 | NA |
| | NGG (SEQ ID NO: 325) | | | | | |
| SP619.TIM3.g17 | ACATCCAGATACTGGCTAAA | 1 | 1 | 2 | 19 | NA |
| | NGG (SEQ ID NO: 326) | | | | | |
| SP619.TIM3.g15 | ATGAAAGGGATGTGAATTAT | 1 | 1 | 2 | 22 | NA |
| | NGG (SEQ ID NO: 327) | | | | | |
| SP619.TIM3.g13 | TGGTGCTCAGGACTGATGAA | 1 | 1 | 2 | 25 | NA |
| | NGG (SEQ ID NO: 328) | | | | | |
| SP619.TIM3.g50 | GGTGTAGAAGCAGGGCAGAT | 1 | 1 | 2 | 36 | NA |
| | NGG (SEQ ID NO: 329) | | | | | |
| SP619.TIM3.g36 | ACGTTGCCACATTCAAACAC | 1 | 1 | 2 | 37 | NA |
| | NGG (SEQ ID NO: 330) | | | | | |
| SP619.TIM3.g45 | ACGAGGTTCCCTGGGGCGGC | 1 | 1 | 2 | 40 | NA |
| | NGG (SEQ ID NO: 331) | | | | | |
| SP619.TIM3.g10 | GCCTGTCCTGTGTTTGAATGN | 1 | 1 | 2 | 47 | NA |
| | GG (SEQ ID NO: 332) | | | | | |
| SP619.TIM3.g9 | GTGCCCGTCTGCTGGGGCAA | 1 | 1 | 2 | 58 | NA |
| | NGG (SEQ ID NO: 333) | | | | | |
| SP619.TIM3.g8 | AACCTCGTGCCCGTCTGCTG | 1 | 1 | 3 | 15 | NA |
| | NGG (SEQ ID NO: 334) | | | | | |
| SP619.TIM3.g48 | GGCGGCTGGGGTGTAGAAGC | 1 | 1 | 3 | 15 | NA |
| | NGG (SEQ ID NO: 335) | | | | | |
| SP619.TIM3.g33 | AGTCACATTCTCTATGGTCAN | 1 | 1 | 3 | 19 | NA |
| | GG (SEQ ID NO: 336) | | | | | |
| SP619.TIM3.g26 | CTGGTTTGATGACCAACTTCN | 1 | 1 | 3 | 21 | NA |
| | GG (SEQ ID NO: 337) | | | | | |
| SP619.TIM3.g29 | CATTCATTATGCCTGGGATTN | 1 | 1 | 3 | 24 | NA |
| | GG (SEQ ID NO: 338) | | | | | |
| SP619.TIM3.g31 | TGCTAGAGTCACATTCTCTAN | 1 | 1 | 3 | 49 | NA |
| | GG (SEQ ID NO: 339) | | | | | |
| SP619.TIM3.g44 | GGGCACGAGGTTCCCTGGGG | 1 | 1 | 3 | 53 | NA |
| | NGG (SEQ ID NO: 340) | | | | | |
| SP619.TIM3.g38 | GGCTCCTTTGCCCCAGCAGA | 1 | 1 | 3 | 58 | NA |
| | NGG (SEQ ID NO: 341) | | | | | |
| SP619.TIM3.g16 | ATTATTGGACATCCAGATAC | 1 | 1 | 3 | 106 | NA |
| | NGG (SEQ ID NO: 342) | | | | | |
| SP619.TIM3.g28 | TTTCATCATTCATTATGCCTN | 1 | 1 | 4 | 23 | NA |
| | GG (SEQ ID NO: 343) | | | | | |
| SP619.TIM3.g4 | TTCTACACCCCAGCCGCCCC | 1 | 1 | 4 | 29 | NA |
| | NGG (SEQ ID NO: 344) | | | | | |
| SP619.TIM3.g34 | TCAGGGACACATCTCCTTTG | 1 | 1 | 4 | 41 | NA |
| | NGG (SEQ ID NO: 345) | | | | | |
| SP619.TIM3.g39 | GCTCCTTTGCCCCAGCAGAC | 1 | 1 | 4 | 42 | NA |
| | NGG (SEQ ID NO: 346) | | | | | |
| SP619.TIM3.g1 | CTCAGAAGTGGAATACAGAG | 1 | 1 | 5 | 35 | NA |
| | NGG (SEQ ID NO: 347) | | | | | |
| SP619.TIM3.g46 | CGAGGTTCCCTGGGGCGGCT | 1 | 2 | 2 | 18 | NA |
| | NGG (SEQ ID NO: 348) | | | | | |
| SP619.TIM3.g37 | GCCACATTCAAACACAGGAC | 1 | 2 | 2 | 25 | NA |
| | NGG (SEQ ID NO: 349) | | | | | |
| SP619.TIM3.g14 | GGTGCTCAGGACTGATGAAA | 1 | 2 | 3 | 28 | NA |
| | NGG (SEQ ID NO: 350) | | | | | |

TABLE 23
Guide RNA (gRNA) Off Target Analysis for hTIM3 (Exon 3)

| Name | gRNA | long_0 | long_1 | long_2 | short_0 | SNP |
|---|---|---|---|---|---|---|
| SP620.TIM3.g1 | AGGTCACCCCTGCACCGA | 1 | 1 | 1 | 4 | rs1036199:0.13 |
| | CTNGG (SEQ ID NO: 351) | | | | | |
| SP620.TIM3.g11 | CTCTCTGCCGAGTCGGTGC | 1 | 1 | 1 | 4 | rs1036199:0.13 |
| | ANGG (SEQ ID NO: 352) | | | | | |
| SP620.TIM3.g10 | TCTCTCTGCCGAGTCGGTG | 1 | 1 | 1 | 6 | rs1036199:0.13 |
| | CNGG (SEQ ID NO: 353) | | | | | |
| SP620.TIM3.g5 | CCAAGGATGCTTACCACC | 1 | 1 | 1 | 8 | NA |
| | AGNGG (SEQ ID NO: 354) | | | | | |
| SP620.TIM3.g12 | TCTCTGCCGAGTCGGTGCA | 1 | 1 | 1 | 9 | rs1036199:0.13 |
| | GNGG (SEQ ID NO: 355) | | | | | |
| SP620.TIM3.g7 | CCCCTGGTGGTAAGCATCC | 1 | 1 | 1 | 10 | NA |
| | TNGG (SEQ ID NO: 356) | | | | | |
| SP620.TIM3.g4 | TCCAAGGATGCTTACCACC | 1 | 1 | 1 | 16 | NA |
| | ANGG (SEQ ID NO: 357) | | | | | |
| SP620.TIM3.g8 | GGTGGTAAGCATCCTTGG | 1 | 1 | 1 | 20 | NA |
| | AANGG (SEQ ID NO: 358) | | | | | |
| SP620.TIM3.g9 | GTGAAGTCTCTCTGCCGAG | 1 | 1 | 2 | 6 | rs1036199:0.13 |
| | TNGG (SEQ ID NO: 359) | | | | | |
| SP620.TIM3.g6 | ATGCTTACCACCAGGGGA | 1 | 1 | 2 | 34 | NA |
| | CANGG (SEQ ID NO: 360) | | | | | |
| SP620.TIM3.g3 | TTCCAAGGATGCTTACCAC | 1 | 1 | 2 | 36 | NA |
| | CNGG (SEQ ID NO: 361) | | | | | |
| SP620.TIM3.g13 | AGTCGGTGCAGGGGTGAC | 1 | 1 | 2 | 45 | NA |
| | CTNGG (SEQ ID NO: 362) | | | | | |
| SP620.TIM3.g2 | ACTTCACTGCAGCCTTTCC | 1 | 1 | 4 | 38 | NA |
| | ANGG (SEQ ID NO: 363) | | | | | |

The gRNA sequences in Table 22 and Table 23 were normalized (% Normalization to NHEJ) for gRNA activity via next generation sequencing (NGS). GFP was used as a control. Following sequencing analysis, the following gRNAs were recommended based on off-target profile: Exon 2: SP619.hTIM3.g12 (45.0%), SP619.hTIM3.g20 (60.9%), and SP619.hTIM3.g49 (45.4%). Exon 3: SP620.hTIM3.g5 (58.0%) and SP620.hTIM3.g7 (2.9%).

All references, patents or applications, U.S. or foreign, cited in the application are hereby incorporated by reference as if written herein in their entireties. Where any inconsistencies arise, material literally disclosed herein controls.

From the foregoing description, one skilled in the art can easily ascertain the essential characteristics of this invention, and without departing from the spirit and scope thereof, can make various changes and modifications of the invention to adapt it to various usages and conditions.

1. A method of making a population of genome-edited immune effector cells, comprising the steps of:
a. editing the genome of a population of T-cell receptor (TCR) bearing immune effector cells;
b. activating the immune effector cell population; and
c. expanding the population of genome-edited immune effector cells.

2. The method as recited in claim 1, wherein the T-cell receptor (TCR) bearing immune effector cells are transduced with at least one chimeric antigen receptor (CAR) that recognize(s) one or more proteins.

3. The method as recited in claim 1, wherein the genome editing step (a) comprises transducing the immune effector cell population with the one or more CARs.

4. The method as recited in claim 1, comprising an additional step to be performed between steps (b) and (c), of transducing the immune effector cell population with the one or more CARs.

5. The method of making a population of genome-edited, chimeric antigen receptor (CAR) bearing immune effector cells, comprising the steps of:
a. editing the genome of a population of T-cell receptor (TCR) bearing immune effector cells;
b. activating the immune effector cell population;
c. transducing the immune effector cell population with at least one chimeric antigen receptor (CAR) that recognize(s) one or more proteins; and
d. expanding the population of genome-edited, chimeric antigen receptor bearing immune effector cells.

6. The method as recited in claim 5, wherein the immune effector cells are purified.

7. The method as recited in claim 6, wherein the immune effector cells are T cells.

8. The method as recited in claim 5, wherein the one or more proteins recognized by the chimeric antigen receptor (CAR) is/are chosen from antigens and cell surface proteins.

9. The method as recited in claim 8, wherein the genome is edited using a CRISPR associated protein (CRISPR/Cas), a transcription activator-like effector nuclease (TALEN), or a zinc-finger nuclease (ZFN).

10. The method as recited in claim 9, wherein the genome is edited using a Cas9 CRISPR associated protein.

11. (canceled)

12. The method as recited in claim 10, wherein the Cas9 is delivered into the cell as mRNA or protein.

13. The method as recited in claim 12, wherein the Cas9 is delivered into the cell as mRNA.

14. The method as recited in claim 12, wherein the Cas9 is delivered into the cell as protein.

15. The method as recited in claim 10, wherein a the Cas9 is delivered contemporaneously with a guide RNA (gRNA) targeting the gene to be edited.

16. The method as recited in claim 14, wherein the delivery is by electroporation.

17. The method as recited in claim 19, wherein the genome editing comprises deleting or suppressing the expression of one or more antigens, cell surface proteins, or secretable proteins.

18.-19. (canceled)

20. The method as recited in claim 17, wherein the deleted or suppressed cell surface protein is the T Cell Receptor (TCR), or a subunit thereof.

21. The method as recited in claim 20, wherein the deleted or suppressed cell surface protein deleted/suppressed is chosen from TRAC (TCR-α), TCR-β, CD3ε, CD3ζ, CD3δ, and CD3γ.

22. The method as recited in claim 21, wherein the deleted or suppressed cell surface protein deleted/suppressed is TRAC.

23. The method as recited in claim 17, wherein the deleted or suppressed cell surface protein deleted/suppressed is a protein which prevents T cell exhaustion.

24.-31. (canceled)

32. The method as recited in claim 17, wherein the deleted or suppressed cell surface protein deleted/suppressed is the target of the CAR(s).

33.-36. (canceled)

37. The method as recited in claim 1, wherein the genome-edited immune effector cells are allowed to rest after editing for between 24 and 48 hours before activation.

38. The method as recited in claim 1, wherein the genome-edited immune effector cells are activated immediately after genome editing.

39. The method as recited in claim 38, wherein the activating of the genome-edited immune effector cells is done by exposing the cell population to anti-CD3 antibodies and anti-CD28 antibodies, or a functional fragment of either of the foregoing.

40. The method as recited in claim 1, wherein the activating of the genome-edited immune effector immune effector cells is done by exposing the cell population to anti-CD3, anti-CD28, and anti-CD2 antibodies, or a functional fragment of either of the foregoing.

41. The method as recited in claim 40, wherein the antibodies are affixed to beads.

42. The method as recited in claim 37, wherein the genome-edited immune effector cells are activated for up to five days.

43.-44. (canceled)

45. The method as recited in claim 40, wherein the anti-CD3 antibodies, anti-CD28 antibodies, and/or anti-CD2 antibodies are removed from the cell population by application of a magnetic field or by washing.

46. The method as recited in claim 5, wherein the CAR is transduced into the cell less than 48 hours post-activation.

47. (canceled)

48. The method as recited in claim 46, wherein the CAR is transduced into the cell using a lentiviral vector encoding the CAR.

49. The method as recited in claim 5, wherein the population of genome-edited immune effector cells is expanded for less than 20 days.

50.-53. (canceled)

54. The method as recited in claim 49, wherein the method is performed at a temperature of between about 25° C. and about 40° C.

55. (canceled)

56. The method as recited in claim 54, comprising the additional step of analyzing the cells by flow cytometry to confirm expression of the at least one chimeric antigen receptor(s).

57. The method as recited in claim 54, comprising the additional step of depleting TCR⁺ cells.

58. The method as recited in claim 5, wherein the immune effector cells to be used are harvested from a healthy donor.

59. The method as recited in claim 58, wherein the donor is a human.

60. The method as recited in claim 56, wherein the at least one chimeric antigen receptor(s) (CARs) specifically bind(s) at least one antigen expressed on a malignant cell.

61.-62. (canceled)

63. The method as recited in claim 60, wherein the antigen expressed on a malignant T cell is chosen from CD2, CD3, CD4, CD5, CD7, TCRA, and TCRβ.

64. The method as recited in claim 56, wherein the at least one chimeric antigen receptor(s) (CARs) specifically bind(s) at least one antigen expressed on a malignant plasma cell.

65. (canceled)

66. The method as recited in claim 56, wherein the at least one chimeric antigen receptor(s) (CARs) specifically bind(s) at least one antigen expressed on a malignant B cell.

67.-68. (canceled)

69. The method as recited in claim 58, wherein the at least one chimeric antigen receptor(s) (CARs) specifically bind(s) at least one antigen expressed on a malignant mesothelial cell.

70. (canceled)

71. A method of making a population of chimeric antigen receptor T (CAR-T) cells in which the CAR targets CD7, in which TRAC and CD7 are deleted (UCART7 cells), comprising the steps of:
a. editing the CD7 and TRAC genes in of a population of T-cells from a healthy human donor to delete/suppress CD7 and TRAC, using a Cas9-CRISPR associated protein and gRNA targeting the gene encoding the one or more antigens(s) or cell surface proteins(s);
b. activating the T cell population;
c. transducing the T cell population with a chimeric antigen receptor that recognizes CD7; and
d. expanding the population of UCART7 cells.

72. A method of making a population of chimeric antigen receptor T (CAR-T) cells in which the CAR is a tandem CAR that targets CD2 and CD3ε, in which CD3ε and CD2 are deleted (tUCART2/3 cells), comprising the steps of:
a. editing the CD2 and CD3ε genes in of a population of T-cells from a healthy human donor to delete/suppress CD2 and CD3ε, using a Cas9-CRISPR associated protein, and gRNA targeting the gene encoding the one or more antigens(s) or cell surface proteins(s);
b. activating the T cell population;
c. transducing the T cell population with a tandem chimeric antigen receptor that recognizes CD2 and CD3ε; and
d. expanding the population of tUCART2/3 cells.

73. A population of genome-edited, chimeric antigen receptor bearing immune effector cells made by the method as recited in claim 1.

74. (canceled)

75. A method of treatment of a solid organ tumor or hematologic malignancy in a patient comprising administering a population of genome-edited, chimeric antigen receptor bearing immune effector cells as recited in claim 1.

76. (canceled)

77. The method as recited in claim 75, wherein the hematologic malignancy is a T-cell malignancy.

78. The method as recited in claim 77, wherein the T cell malignancy is T-cell acute lymphoblastic leukemia (T-ALL).

79. The method as recited in claim 77, wherein the T cell malignancy is non-Hodgkin's lymphoma.

80. The method as recited in claim 75, wherein the hematologic malignancy is a B-cell malignancy.

81. The method as recited in claim 80, wherein the B-cell malignancy is a B cell lymphoma.

82. The method as recited in claim 80, wherein the B-cell malignancy is a B cell leukemia.

83. The method as recited in claim 75, wherein the hematologic malignancy is a myeloid malignancy.

84. The method as recited in claim 75, wherein the hematologic malignancy is acute myeloid leukemia (AML).
